(19) United States
(12) Patent Application Publication
(10) Pub. No.: US 2026/0209868 A1
(43) Pub. Date: Jul. 23, 2026

(54) MAIZE PLANTS COMPRISING RESISTANCE TO SOUTHERN LEAF BLIGHT AND COMPOSITIONS AND METHODS FOR SELECTING AND PRODUCING THE SAME

(71) Applicants: PIONEER HI-BRED INTERNATIONAL, INC., JOHNSTON, IA (US); HENAN AGRICULTURAL UNIVERSITY, ZHENGZHOU (CN)

(72) Inventors: JUNQINAG DING, ZHENGZHOU (CN); XUEYING LI, ZHENGZHOU (CN); XIAODI ZHAO, ZHENGZHOU (CN); CE DENG, ZHENGZHOU (CN); WENJIE DU, ZHENGZHOU (CN); TANGSHUN AI, ZHENGZHOU (CN); APRIL LEONARD, DES MOINES, IA (US); BALIN LI, JOHNSTON, IA (US); JIA JUN LONG, DES MOINES, IA (US); SHAWN THATCHER, URBANDALE, IA (US); GIRMA M. TABOR, JOHNSTON, IA (US)

(73) Assignees: PIONEER HI-BRED INTERNATIONAL, INC., JOHNSTON, IA (US); HENAN AGRICULTURAL UNIVERSITY, ZHENGZHOU, HA (CN)

(21) Appl. No.: 19/138,801

(22) PCT Filed: Dec. 15, 2023
(86) PCT No.: PCT/US23/84377

§ 371 Date: Jun. 13, 2025

(30) Foreign Application Priority Data
Dec. 16, 2022 (WO) PCT/CN2022/139436

(51) Int. Cl. C12Q 1/6895; C12N 15/82

(52) U.S. Cl. CPC C12Q 1/6895; C12N 15/8282; C12Q 2600/13; C12Q 2600/156

(57) ABSTRACT

The present application is related to markers which have been determined to be genetically linked to a locus which provides resistance to southern leaf blight (SLB). Maize plants comprising newly conferred or increased resistance to SLB, relative to control plants, are also provided herein.

This application claims the benefit PCT Application Number PCT/CN2022/0139436 filed on Dec. 16, 2022 and PCT Application Number PCT/US2023/084377 filed on Dec. 15, 2023. The entire disclosure of each is incorporated herein by reference.

### FIELD

The field includes plants, plant genetics, plant breeding, and methods of selecting and producing plants comprising resistance to southern leaf blight. The field also includes disease resistant plants and related compositions.

### REFERENCE TO SEQUENCE LISTING SUBMITTED ELECTRONICALLY

The official copy of the sequence listing is submitted electronically with this application as an XML file named “117927-US-PCT-1 Sequence Listing” created on Jun. 11, 2025. The XML file has a size of 77,281 bytes and is filed concurrently with this specification. The sequence listing contained in this XML file document is part of the specification and is herein incorporated by reference in its entirety.

### BACKGROUND

Southern leaf blight (SLB) is a common fungal disease of maize plants caused by the fungal pathogen Bipolaris maydis (also known as Cochliobolus heterostrophus in its teleomorph state). SLB has been a major concern to farmers and grain producers due to its potential for reducing yields and/or quality of maize crops. Subtypes of the disease agent include Races O, T, and C. Lesions on the leaf are always present in infected plants, but lesions may be present on other parts of the plant as well. SLB can survive on plant debris from one season and be transmitted to newly planted corn, the following season. SLB spores can quickly germinate and spread under favorable conditions. While management and mitigation techniques for minimizing the impact of SLB on maize crops exist, there remains a need for additional development of new techniques for management and minimizing the potential impact of SLB.

### SUMMARY

In one aspect, the present disclosure provides a method of screening for a maize plant comprising a marker allele associated with resistance to southern leaf blight (SLB). The method comprises detecting, in nucleic acid from a maize plant, at least one of the following marker alleles on chromosome 3: a “C” at marker D6, corresponding to position 51 of SEQ ID NO: 1; a “C” at marker C10, corresponding to position 51 of SEQ ID NO: 2; a “C” at marker A10, corresponding to position 51 of SEQ ID NO: 3; a “G” at marker G6, corresponding to position 51 of SEQ ID NO: 4; a “G” at marker H6, corresponding to position 51 of SEQ ID NO: 5; a “T” at marker D10, corresponding to position 51 of SEQ ID NO: 6; a “C” at marker F10, corresponding to position 51 of SEQ ID NO: 7; an “A” at marker K6, corresponding to position 51 of SEQ ID NO: 8; a “C” at marker L6, corresponding to position 51 of SEQ ID NO: 9; a “T” at marker O6, corresponding to position 51 of SEQ ID NO: 10; a “G” at marker Q6, corresponding to position 51 of SEQ ID NO: 11; a “C” at marker G3, corresponding to position 51 of SEQ ID NO: 12; a “T” at marker D7, corresponding to position 51 of SEQ ID NO: 13; a “T” at marker L4, corresponding to position 51 of SEQ ID NO: 14; a “T” at marker E7, corresponding to position 51 of SEQ ID NO: 15; an “A” at marker G7, corresponding to position 51 of SEQ ID NO: 16; a “G” at marker H7, corresponding to position 51 of SEQ ID NO: 17; a “T” at marker I7, corresponding to position 51 of SEQ ID NO: 18; a “T” at marker J7, corresponding to position 51 of SEQ ID NO: 19; a “G” at marker K7, corresponding to position 51 of SEQ ID NO: 20; an “A” at marker L7, corresponding to position 51 of SEQ ID NO: 21; a “T” at marker M7, corresponding to position 51 of SEQ ID NO: 22; an “A” at marker N7, corresponding to position 51 of SEQ ID NO: 23; or an “A” at marker O4, corresponding to position 51 of SEQ ID NO: 24. The method can further comprise obtaining a nucleic acid sample from each of one or more plants, seeds, tissues or germplasm and screening the sample for one or more of the foregoing marker alleles; wherein the presence of the one or more marker alleles is associated with resistance to SLB.

In some examples, the foregoing method includes obtaining a nucleic acid sample from each of a plurality of maize plants (or seeds, tissues or germplasm thereof); screening each sample and identifying a sample comprising one or more of the foregoing marker alleles associated with resistance to SLB. The method can further comprise selecting a maize plant (or seed, tissue, or germplasm thereof) that comprises the at least one marker.

In further examples, the method comprises crossing the selected maize plant with a second maize plant; and obtaining a progeny plant that comprises at least one of the foregoing marker alleles associated with resistance to SLB. For example, the selected maize plant can comprise a QTL or chromosomal interval flanked by and including the markers D6, corresponding to a “C” at position 51 of SEQ ID NO: 1 and G3, corresponding to a “C” at position 51 of SEQ ID NO: 12. In some cases, the selected maize plant comprises a QTL allele or a chromosomal interval flanked by and including the markers D7, corresponding to a “T” at position 51 of SEQ ID NO: 13 and O4, corresponding to an “A” at position 51 of SEQ ID NO: 24. In some examples, one or more of markers D6, C10, A10, G6, H6, D10, F10, K6, L6, O6, Q6, and G3 are detected; or one or more of markers D7, L4, E7, G7, H7, I7, J7, K7, L7, M7, N7, and O4 are detected; or each of markers D6, C10, A10, G6, H6, D10, F10, K6, L6, O6, Q6, and G3 are detected; or each of markers D7, L4, E7, G7, H7, I7, J7, K7, L7, M7, N7, and O4.

In another aspect, provided herein is a method of producing a maize plant comprising a QTL allele associated with resistance to southern leaf blight. The method comprises screening a first maize plant population for a QTL associated with resistance to southern leaf blight. The QTL comprises one or more of marker alleles: a “C” at D6, corresponding to position 51 of SEQ ID NO: 1; a “C” at C10, corresponding to position 51 of SEQ ID NO: 2; a “C” at A10, corresponding to position 51 of SEQ ID NO: 3; a “G” at G6, corresponding to position 51 of SEQ ID NO: 4; a “G” at H6, corresponding to position 51 of SEQ ID NO: 5; a “T” at D10, corresponding to position 51 of SEQ ID NO: 6; a “C” at F10, corresponding to position 51 of SEQ ID NO: 7; an “A” at K6, corresponding to position 51 of SEQ ID NO: 8; a “C” at L6, corresponding to position 51 of SEQ ID NO: 9; a “T” at O6, corresponding to position 51 of SEQ ID NO: 10; a “G” at Q6, corresponding to position 51 of SEQ ID NO: 11; or a “C” at G3, corresponding to position 51 of SEQ ID NO: 12. For example, the QTL can comprise 2, 3, 4, 5, 6, 7, 8, 9, 10, 11, or all 12 of each of the foregoing marker alleles.

The QTL can alternatively comprises a “T” at D7, corresponding to position 51 of SEQ ID NO: 13; a “T” at L4, corresponding to position 51 of SEQ ID NO: 14; a “T” at E7, corresponding to position 51 of SEQ ID NO: 15; an “A” at G7, corresponding to position 51 of SEQ ID NO: 16; a “G” at H7, corresponding to position 51 of SEQ ID NO: 17; a “T” at I7, corresponding to position 51 of SEQ ID NO: 18; a “T” at J7, corresponding to position 51 of SEQ ID NO: 19; a “G” at K7, corresponding to position 51 of SEQ ID NO: 20; an “A” at L7, corresponding to position 51 of SEQ ID NO: 21; a “T” at M7, corresponding to position 51 of SEQ ID NO: 22; an “A” at N7, corresponding to position 51 of SEQ ID NO: 23; and an “A” at O4, corresponding to position 51 of SEQ ID NO: 24. For example the QTL can comprise 2, 3, 4, 5, 6, 7, 8, 9, 10, 11, or all 12 of each of the foregoing marker alleles.

The method of producing a maize plant can also comprise selecting from the population at least one maize plant comprising the QTL allele; crossing the selected maize plant with a second maize plant; and obtaining a progeny plant that comprises the QTL allele. In some examples, the method comprises introgressing the QTL allele into a second maize plant population. In some examples, the QTL allele is located in a chromosomal interval flanked by and including the markers D6, corresponding to a “C” at position 51 of SEQ ID NO: 1, and G3, corresponding to a “C” at position 51 of SEQ ID NO: 12. In other examples, the QTL allele is located in a chromosomal interval flanked by and including the markers D7, corresponding to a “T” at position 51 of SEQ ID NO: 13 and O4, corresponding to an “A” at position 51 of SEQ ID NO: 24. In some examples, this method comprises detecting and selecting a plant comprising one or more of markers D6, C10, A10, G6, H6, D10, F10, K6, L6, O6, Q6, and G3. In some examples, this method comprises detecting and selecting a plant comprising one or more of markers D7, L4, E7, G7, H7, I7, J7, K7, L7, M7, N7, and O4. In some examples, this method comprises detecting and selecting a plant comprising each of markers D6, C10, A10, G6, H6, D10, F10, K6, L6, O6, Q6, and G3 are detected. In some examples, this method comprises detecting and selecting a plant comprising each of markers D7, L4, E7, G7, H7, I7, J7, K7, L7, M7, N7, and O4 are detected.

In another aspect, provided herein is a method of selecting a maize plant comprising resistance to southern leaf blight. The method comprises screening a population of maize plants for the presence of a gene encoding a protein comprising at least 95%, at least 96%; at least 97%, at least 98% or at least 99% amino acid sequence identity to one or more of SEQ ID NOs: 27, 30, or 33 and selecting a maize plant comprising the gene. In some examples, the protein comprises 100% amino acid sequence identity to SEQ ID NO: 27, 30, or 33. In some examples, the method comprises crossing the selected maize plant with a second maize plant and obtaining a progeny plant that comprises the gene. In some examples, the protein is encoded by a gene comprising a nucleotide sequence with at least 95% %, at least 96%; at least 97%, at least 98% or at least 99% nucleotide sequence identity to one or more of SEQ ID NO: 25, SEQ ID NO: 26, SEQ ID NO: 28, SEQ ID NO: 29, SEQ ID NO: 31, or SEQ ID NO: 32. In some examples, the protein is encoded by a gene comprising a nucleotide sequence comprising the sequence of SEQ ID NO: 25, SEQ ID NO: 26, SEQ ID NO: 28, SEQ ID NO: 29, SEQ ID NO: 31, or SEQ ID NO: 32.

In another aspect, provided herein is a method for producing a recombinant maize plant cell. The method comprises introducing a heterologous P450 gene sequence into the genome of a maize plant cell and selecting a plant cell comprising the heterologous P450 gene sequence encoded in the plant cell's genome. The method can comprise introducing a site-specific modification of endogenous P450 to thereby introduce a gene edited P450 nucleotide sequence into the plant cell. The site-specific modification can be induced by a CRISPR-associated endonuclease. In some examples, the method further comprises growing a plant from the plant cell comprising the gene edited P450 gene sequence. In certain examples, this plant exhibits increased resistance to southern leaf blight. In some examples, the method comprises introducing a heterologous P450 transgene sequence that replaces the endogenous P450 gene. In other examples, the P450 transgene sequence is inserted into a locus different from the endogenous P450 locus. The foregoing gene-edited P450 gene sequence or P450 transgene can encode a protein comprising an amino acid sequence at least 95%, at least 96%; at least 97%, at least 98% or at least 99% identical to one or more of SEQ ID NO: 27, 30, or 33; e.g., a protein comprising the amino acid sequence of SEQ ID NO: 27, 30, or 33. In some examples, the heterologous P450 gene sequence or P450 transgene comprises a nucleotide sequence comprising at least 95%, at least 96%; at least 97%, at least 98% or at least 99% identity to one or more of SEQ ID NO: 25, SEQ ID NO: 26, SEQ ID NO: 28, SEQ ID NO: 29, SEQ ID NO: 31, or SEQ ID NO: 32; e.g., the nucleotide sequence of SEQ ID NO: 25, SEQ ID NO: 26, SEQ ID NO: 28, SEQ ID NO: 29, SEQ ID NO: 31, or SEQ ID NO: 32. Methods for gene editing and introducing transgenes are described herein.

In another aspect, the present disclosure provides a recombinant plant comprising a heterologous gene encoding a protein comprising at least 95%, at least 96%; at least 97%, at least 98% or at least 99% amino acid sequence identity to one or more of SEQ ID NOs: 27, 30, or 33. In some examples, the protein comprises 100% amino acid sequence identity to SEQ ID NO: 27, 30, or 33. In some examples, the gene comprises a nucleotide sequence comprising at least 95%, at least 96%; at least 97%, at least 98% or at least 99% nucleotide sequence identity to one or more of SEQ ID NO: 25, SEQ ID NO: 26, SEQ ID NO: 28, SEQ ID NO: 29, SEQ ID NO: 31, or SEQ ID NO: 32. In some examples, the gene comprises the nucleotide sequence of SEQ ID NO: 25, SEQ ID NO: 26, SEQ ID NO: 28, SEQ ID NO: 29, SEQ ID NO: 31, or SEQ ID NO: 32.

In another aspect, the present disclosure provides a recombinant plant seed comprising a heterologous gene encoding a protein comprising at least 95%, at least 96%; at least 97%, at least 98% or at least 99% amino acid sequence identity to one or more of SEQ ID NOs: 27, 30, or 33. In some examples, the protein comprises 100% amino acid sequence identity to SEQ ID NO: 27, 30, or 33. In some examples, the gene comprises a nucleotide sequence comprising at least 95%, at least 96%; at least 97%, at least 98% or at least 99% nucleotide sequence identity to one or more of SEQ ID NO: 25, SEQ ID NO: 26, SEQ ID NO: 28, SEQ ID NO: 29, SEQ ID NO: 31, or SEQ ID NO: 32. In some examples, the gene comprises the nucleotide sequence of SEQ ID NO: 25, SEQ ID NO: 26, SEQ ID NO: 28, SEQ ID NO: 29, SEQ ID NO: 31, or SEQ ID NO: 32.

### BRIEF DESCRIPTION OF THE SEQUENCE LISTING

The disclosure can be more fully understood from the following detailed description and the sequence listing which forms a part of this application.

The sequence descriptions and sequence listing attached hereto comply with the rules governing nucleotide and/or amino acid sequence disclosures in patent applications as set forth in 37 C.F.R. §§ 1.831-1.834 and in the WIPO ST.26 Standard. The symbols and format used for nucleotide and amino acid sequence data comply with the rules set forth in 37 C.F.R. § 1.832. Unless stated otherwise, the case of individual letters representing amino acid or nucleotide residues does not have any significance (e.g., “atgc” is the same as “ATGC”, and “MRYG” is the same as “mryg”. Although only one strand is shown, each disclosed nucleotide sequence should be understood to encompass its complementary strand (i.e., reverse complement sequence).

SEQ ID NO: 1 is the D6 marker sequence, with the SNP at position 51 indicated as a “y”, meaning “c or t”. The SNP linked with resistance to SLB comprises a “c” at position 51.

SEQ ID NO: 2 is the C10 marker sequence, with the SNP at position 51 indicated as a “y”, meaning “c or t”. The SNP linked with resistance to SLB comprises a “c” at position 51.

SEQ ID NO: 3 is the A10 marker sequence, with the SNP at position 51 indicated as a “y”, meaning “c or t”. The SNP linked with resistance to SLB comprises a “c” at position 51.

SEQ ID NO: 4 is the G6 marker sequence, with the SNP at position 51 indicated as an “s”, meaning “g or c”. The SNP linked with resistance to SLB comprises a “g” at position 51.

SEQ ID NO: 5 is the H6 marker sequence, with the SNP at position 51 indicated as an “r”, meaning “g or a”. The SNP linked with resistance to SLB comprises a “g” at position 51.

SEQ ID NO: 6 is the D10 marker sequence, with the SNP at position 51 indicated as a “y”, meaning “t or c”. The SNP linked with resistance to SLB comprises a “t” at position 51.

SEQ ID NO: 7 is the F10 marker sequence, with the SNP at position 51 indicated as an “m”, meaning “c or a”. The SNP linked with resistance to SLB comprises a “c” at position 51.

SEQ ID NO: 8 is the K6 marker sequence, with the SNP at position 51 indicated as an “m”, meaning “a or c”. The SNP linked with resistance to SLB comprises an “a” at position 51.

SEQ ID NO: 9 is the L6 marker sequence, with the SNP at position 51 indicated as a “y”, meaning “c or t”. The SNP linked with resistance to SLB comprises a “c” at position 51.

SEQ ID NO: 10 is the O6 marker sequence, with the SNP at position 51 indicated as a “y”, meaning “t or c”. The SNP linked with resistance to SLB comprises a “t” at position 51.

SEQ ID NO: 11 is the Q6 marker sequence, with the SNP at position 51 indicated as a “k”, meaning “g or t”. The SNP linked with resistance to SLB comprises a “g” at position 51.

SEQ ID NO: 12 is the G3 marker sequence, with the SNP at position 51 indicated as a “y”, meaning “c or t”. The SNP linked with resistance to SLB comprises a “C” at position 51.

SEQ ID NO: 13 is the D7 marker sequence, with the SNP at position 51 indicated as a “y”, meaning “t or c”. The SNP linked with resistance to SLB comprises a “t” at position 51.

SEQ ID NO: 14 is the L4 marker sequence, with the SNP at position 51 indicated as a “w”, meaning “t or a”. The SNP linked with resistance to SLB comprises a “t” at position 51.

SEQ ID NO: 15 is the E7 marker sequence, with the SNP at position 51 indicated as a “y”, meaning “t or c”. The SNP linked with resistance to SLB comprises a “t” at position 51.

SEQ ID NO: 16 is the G7 marker sequence, with the SNP at position 51 indicated as an “r”, meaning “a or g”. The SNP linked with resistance to SLB comprises an “a” at position 51.

SEQ ID NO: 17 is the H7 marker sequence, with the SNP at position 51 indicated as a “k”, meaning “g or t”. The SNP linked with resistance to SLB comprises a “g” at position 51.

SEQ ID NO: 18 is the I7 marker sequence, with the SNP at position 51 indicated as a “y”, meaning “t or c”. The SNP linked with resistance to SLB comprises an “t” at position 51.

SEQ ID NO: 19 is the J7 marker sequence, with the SNP at position 51 indicated as a “y”, meaning “t or c”. The SNP linked with resistance to SLB comprises an “t” at position 51.

SEQ ID NO: 20 is the K7 marker sequence, with the SNP at position 51 indicated as an “r”, meaning “g or a”. The SNP linked with resistance to SLB comprises an “g” at position 51.

SEQ ID NO: 21 is the L7 marker sequence, with the SNP at position 51 indicated as an “r”, meaning “a or g”. The SNP linked with resistance to SLB comprises an “a” at position 51.

SEQ ID NO: 22 is the M7 marker sequence, with the SNP at position 51 indicated as a “y”, meaning “t or c”. The SNP linked with resistance to SLB comprises an “t” at position 51.

SEQ ID NO: 23 is the N7 marker sequence, with the SNP at position 51 indicated as an “r”, meaning “a or g”. The SNP linked with resistance to SLB comprises an “a” at position 51.

SEQ ID NO: 24 is the O4 marker sequence, with the SNP at position 51 indicated as a “w”, meaning “a or t”. The SNP linked with resistance to SLB comprises an “a” at position 51.

SEQ ID NO: 25 is the nucleic acid genomic sequence for the P450 gene of CIMBL83. This sequence includes the endogenous promoter and terminator regions.

SEQ ID NO: 26 is the nucleic acid coding sequence (e.g., cDNA) encoding the P450 protein of CIMBL83.

SEQ ID NO: 27 is the amino sequence encoded by the P450 gene of CIMBL83.

SEQ ID NO: 28 is the nucleic acid genomic sequence for the P450 gene of CML304. This sequence includes the endogenous promoter and terminator regions.

SEQ ID NO: 29 is the nucleic acid coding sequence (e.g., cDNA) encoding the P450 protein of CML304.

SEQ ID NO: 30 is the amino sequence encoded by the P450 gene of CML304.

SEQ ID NO: 31 is the nucleic acid genomic sequence for the P450 gene of K22. This sequence includes the endogenous promoter and terminator regions.

SEQ ID NO: 32 is the nucleic acid coding sequence (e.g., cDNA) encoding the P450 protein of K22.

SEQ ID NO: 33 is the amino sequence encoded by the P450 gene of K22.

SEQ ID NO: 34 is the G8 marker sequence, with the SNP at position 51 indicated as a “r”, meaning “a or g”. The SNP linked with resistance to SLB comprises an “a” at position 51.

### DETAILED DESCRIPTION

The present disclosure provides markers which have been determined to be genetically linked to a locus which provides resistance to SLB. The present disclosure also provides methods using the markers to select resistant plants or to counter-select susceptible plants. Maize plants comprising newly conferred or increased resistance to SLB, relative to control plants, are also provided herein. Causal genes which provide the newly conferred or increased resistance when expressed in the plants and plants expressing the same are also provided.

### Definitions

The following definitions are provided as an aid to understand the present disclosure.

The disclosure is not limited to particular examples, which can, of course, vary. The terminology and exemplary examples used herein are for the purpose of describing particular aspects of the disclosure only and are not intended to be limiting. As used herein, terms in the singular and the singular forms “a”, “an” and “the”, for example, include plural referents unless the context clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Unless otherwise indicated, nucleic acids are written left to right in 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the disclosure pertains. In describing and claiming the subject matter of the current disclosure, the following terminology will be used in accordance with the definitions set out below.

The term “allele” refers to one of two or more different nucleotide sequences that occur at a specific locus.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

The term “assemble” applies to BACs and their propensities for coming together to form contiguous stretches of DNA. A BAC “assembles” to a contig based on sequence alignment, if the BAC is sequenced, or via the alignment of its BAC fingerprint to the fingerprints of other BACs. Public assemblies can be found using the Maize Genome Browser, which is publicly available on the internet.

An allele is “associated with” a trait when it is part of or linked to a DNA sequence that affects the expression of the trait. The presence of the allele is an indicator of how the trait will be expressed. For example, an allele can be linked to resistance to SLB if the allele is part of or linked to a gene that can provide resistance to SLB.

A “BAC”, or bacterial artificial chromosome, is a cloning vector derived from the naturally occurring F factor of Escherichia coli, which itself is a DNA element that can exist as a circular plasmid or can be integrated into the bacterial chromosome. BACs can accept large inserts of DNA sequence. In maize, a number of BACs each containing a large insert of maize genomic DNA from maize inbred line B73, have been assembled into contigs (overlapping contiguous genetic fragments, or “contiguous DNA”), and this assembly is available publicly on the internet.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

A “chromosome” is a single piece of coiled DNA containing many genes that act and move as a unity during cell division and therefore can be said to be linked. It can also be referred to as a “linkage group”.

The term “contiguous DNA” refers to an uninterrupted stretch of genomic DNA represented by partially overlapping pieces or contigs.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

A plant referred to herein as “diploid” has two sets of chromosomes.

A plant referred to herein as a “doubled haploid” is developed by doubling the haploid set of chromosomes (i.e., half the normal number of chromosomes). A doubled haploid plant has two identical sets of chromosomes, and all loci are considered homozygous.

An “elite line” is any line that has resulted from breeding and selection for superior agronomic performance.

An “exotic maize strain” or an “exotic maize germplasm” is a strain derived from a maize plant not belonging to an available elite maize line or strain of germplasm. In the context of a cross between two maize plants or strains of germplasm, an exotic germplasm is not closely related by descent to the elite germplasm with which it is crossed. Most commonly, the exotic germplasm is not derived from any known elite line of maize, but rather is selected to introduce novel genetic elements (typically novel alleles) into a breeding program.

A “favorable allele” is the allele at a particular locus that confers, or contributes to, an agronomically desirable phenotype, e.g., SLB resistance. A favorable allele of a marker is a marker allele that segregates with (e.g., is linked to) the favorable phenotype.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g., markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency.

“Genetic markers”, “markers”, or “molecular markers” are nucleic acids that are polymorphic in a population and where the alleles of which can be detected and distinguished by one or more analytic methods, described below. The terms also refer to nucleic acid sequences complementary to the genomic sequences, such as nucleic acids used as probes. Markers corresponding to genetic polymorphisms between members of a population can be detected by analytic methods such as, for example, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), detection of amplified fragment length polymorphisms (AFLPs), or next generation sequencing techniques. Other appropriate methods include detection of expressed sequence tags (ESTs) and SSR markers derived from EST sequences and randomly amplified polymorphic DNA (RAPD). In addition to nucleic acids, “Genetic markers”, “markers”, or “molecular markers” may also refer to proteins or inherited phenotypes whose causal genes are known or expected to be linked to a favorable allele.

For example, markers are provided herein, such as the SNPs in Table 1, which are linked to a favorable allele providing SLB resistance. As used herein, “Marker allele” refers to the particular sequence at the marker locus that is linked to the favorable allele.

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by an individual.

The term “genotype” is the genetic constitution of an individual (or group of individuals) at one or more genetic loci. Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

A plant referred to as “haploid” has a single set (genome) of chromosomes.

A “haplotype” is the genotype of an individual at a plurality of genetic loci, i.e., a combination of alleles. Typically, the genetic loci described by a haplotype are physically and genetically linked, i.e., on the same chromosome segment.

The term “heterogeneity” is used to indicate that individuals within the group differ in genotype at one or more specific loci.

The heterotic response of material, or “heterosis”, can be defined by performance which exceeds the average of the parents (or high parent) when crossed to other dissimilar or unrelated groups.

An individual is “heterozygous” if more than one allele type is present at a given locus (e.g., a diploid individual with one copy each of two different alleles).

The term “homogeneity” indicates that members of a group have the same genotype at one or more specific loci.

An individual is “homozygous” if the individual has only one type of allele at a given locus (e.g., a diploid individual has a copy of the same allele at a locus for each of two homologous chromosomes).

The term “hybrid” refers to the progeny obtained between the crossing of at least two genetically dissimilar parents.

“Hybridization” or “nucleic acid hybridization” refers to the pairing of complementary RNA and DNA strands as well as the pairing of complementary DNA single strands.

The term “hybridize” means to form base pairs between complementary regions of nucleic acid strands.

An “IBM genetic map” can refer to any of following maps: IBM, IBM2, IBM2 neighbors, IBM2 FPC0507, IBM2 2004 neighbors, IBM2 2005 neighbors, IBM2 2005 neighbors frame, IBM2 2008 neighbors, IBM2 2008 neighbors frame, or the latest version on the maizeGDB website. IBM genetic maps are based on a B73×Mo17 population in which the progeny from the initial cross were random-mated for multiple generations prior to constructing recombinant inbred lines for mapping. Newer versions reflect the addition of genetic and BAC mapped loci as well as enhanced map refinement due to the incorporation of information obtained from other genetic maps or physical maps, cleaned date, or the use of new algorithms.

The term “inbred” refers to a line that has been bred for genetic homogeneity.

The term “indel” refers to an insertion or deletion, wherein one line may be referred to as comprising an inserted nucleotide or piece of DNA relative to a second line, or the second line may be referred to as comprising a deletion of a nucleotide or piece of DNA relative to the first line.

“Introducing”, as used herein, means presenting to the plant or plant cell the polynucleotide or polypeptide in such a manner that the sequence gains access to the interior of a cell. The methods of the present disclosure do not depend on a particular method for introducing a polynucleotide or polypeptide into a plant, only that the polynucleotide(s) or polypeptide(s) gains access to the interior of at least one cell of the plant. Methods for introducing polynucleotide(s) or polypeptide(s) into plants include, but are not limited to, stable transformation methods, transient transformation methods, and virus-mediated methods.

“Stable transformation” as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant, or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells and pollen).

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840), direct gene transfer (Paszkowski et al. (1984) EMBO J. 3:2717-2722) and ballistic particle acceleration (see, for example, U.S. Pat. Nos. 4,945,050; 5,879,918; 5,886,244 and 5,932,782; Tomes et al. (1995) in Plant Cell, Tissue, and Organ Culture: Fundamental Methods, ed. Gamborg and Phillips (Springer-Verlag, Berlin) and McCabe et al. (1988) Biotechnology 6:923-926) and LecI transformation (WO 00/28058). For potato transformation see, Tu et al. (1998) Plant Molecular Biology 37:829-838 and Chong et al. (2000) Transgenic Research 9:71-78. Additional transformation procedures can be found in Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783 and 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens).

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line comprising a desired genetic background and selected for the desired allele, to result in the allele becoming fixed in a selected genetic background.

The process of “introgressing” is often referred to as “backcrossing” when the sexual cross process is repeated two or more times.

“Backcrossing” refers to the process whereby hybrid progeny are crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being backcrossed. Repeated backcrossing can result in “introgression” of the allele into the recipient or recurrent parent line. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F₁ generation; the term “BC₁” then refers to the second use of the recurrent parent, “BC₂” refers to the third use of the recurrent parent, and so on. Similarly, “BC5F2” refers to the second generation produced by the sixth use of the recurrent parent.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor.

As used herein, the term “linkage” is used to describe the degree with which one marker locus is associated with another marker locus, a favorable allele, or some other locus. The linkage relationship between a molecular marker and a locus affecting a phenotype (e.g., a favorable allele) is given as a “probability” or “adjusted probability”. Linkage can be expressed as a desired limit or range. For example, in some examples, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g., an F₂; the IBM2 maps consist of multiple meioses). In some aspects, it is advantageous to define a bracketed range of linkage, for example, between 10 and 20 cM, between 10 and 30 CM, or between 10 and 40 cM. The more closely a marker is linked to a second locus, the better an indicator for the second locus that marker becomes.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype.

Linkage disequilibrium is most commonly assessed using the measure r², which is calculated using the formula described by Hill, W. G. and Robertson, A, Theor. Appl. Genet. 38:226-231 (1968). When r²=1, complete LD exists between the two marker loci, meaning that the markers have not been separated by recombination and have the same allele frequency. The r² value will be dependent on the population used. Values for r² above ⅓ indicate sufficiently strong LD to be useful for mapping (Ardlie et al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

“Maize” refers to a plant of the Zea mays and is also known as “corn”.

The term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

“Marker assisted selection” (of MAS) is a process by which individual plants are selected based on marker genotypes.

“Marker assisted counter-selection” is a process by which marker genotypes are used to identify plants that will not be selected, allowing them to be removed from a breeding program or planting.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

An allele “negatively” correlates with a trait when it is linked to it and when presence of the allele is an indicator that a desired trait or trait form will not occur in a plant comprising the allele.

A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant.

A “polymorphism” is a variation in the DNA between two or more individuals within a population. A polymorphism preferably has a frequency of at least 1% in a population. A useful polymorphism can include an SNP, a simple sequence repeat (SSR), or an insertion/deletion polymorphism, also referred to herein as an “indel”.

An allele “positively” correlates with a trait when it is linked to it and when presence of the allele is an indicator that the desired trait or trait form will occur in a plant comprising the allele.

The term “progeny” refers to the offspring generated from a cross.

A “progeny plant” is a plant generated from a cross between two plants.

The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the nucleic acid sequence (e.g., gene or genes) that affect the trait in question.

As used herein, a “recombinant” plant or plant cell, or a “recombinant” nucleic acid or protein/peptide, is a plant, plant cell, nucleic acid, or protein/peptide comprising a heterologous nucleic acid sequence (which encodes a heterologous protein/peptide or a noncoding RNA). As used herein “heterologous” means that a given nucleic acid sequence has been located in a genome, locus, or construct through human intervention. The heterologous sequence may be naturally occurring, but now located in a genome, locus, or construct where the sequence is not naturally found. Alternatively, the sequence may be “heterologous” in that it is located in a genome, locus, or construct and is non-naturally occurring. For example, a P450 gene may naturally occur in one plant, but be “heterologous” in that the gene is now encoded in a plant genome that previously did not encode the gene or encoded a different allele of the gene. A heterologous gene can be inserted into a genome via, for example, transformation and/or site-specific nuclease-based methods.

A “reference sequence” or a “consensus sequence” is a defined sequence used as a basis for sequence comparison. A reference sequence for a marker can be obtained by sequencing a number of lines at the locus, aligning the nucleotide sequences in a sequence alignment program (e.g., Sequencher), and then obtaining the most common nucleotide sequence of the alignment. Polymorphisms found among the individual sequences are annotated within the consensus sequence. A reference sequence is not usually an exact copy of any individual DNA sequence but represents an amalgam of available sequences and is useful for designing primers and probes to polymorphisms within the sequence.

As used herein, “southern leaf bight (SLB) resistance” refers to increased resistance or tolerance to a fungal pathogen that causes SLB when compared to a control plant comprising less resistance. Resistance effects may vary from a slight increase in tolerance to the effects of the fungal pathogen (e.g., partial inhibition of pathogenesis) to total resistance such that the plant is unaffected by the presence of the fungal pathogen. The examples of the disclosure provide materials and methods that will increase resistance to the fungal pathogen that causes SLB.

A “topcross test” is a test performed by crossing each individual (e.g., a selection, inbred line, clone or progeny individual) with the same pollen parent or “tester”, usually a homozygous line.

The phrase “under stringent conditions” refers to conditions under which a probe or polynucleotide will hybridize to a specific nucleic acid sequence, typically in a complex mixture of nucleic acids, but to essentially no other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C., depending on primer length. Additional guidelines for determining hybridization parameters are provided in numerous references.

An “unfavorable allele” of a marker is a marker allele that segregates with the unfavorable plant phenotype, therefore providing the benefit of identifying plants that can be removed from a breeding program or planting.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics”, “agronomic traits”, and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, WI). Unless stated otherwise, multiple alignment of the sequences provided herein were performed using the CLUSTAL V method of alignment (Higgins and Sharp, CABIOS. 5:151 153 (1989)) with the default parameters (GAP PENALTY=10, GAP LENGTH PENALTY=10). Default parameters for pairwise alignments and calculation of percent identity of protein sequences using the CLUSTAL V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED-4. After alignment of the sequences, using the CLUSTAL V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

### QTL Locations and Causative Genes

The present disclosure provides a QTL on maize chromosome 3 that was identified as being associated with SLB resistance (see Example 1). The QTL was named qSLB3.1. Three different inbred lines were used as donor plants in the work described herein—CIMBL83, CML304, and K22. Markers for the QTL of CIMBL83 and CML304 are located on maize chromosome 3 in an interval flanked by and including the D6 marker and the G3 marker. Markers for the QTL of K22 are located on maize chromosome 3 in an interval flanked by and including the D7 marker and the O4 marker. The markers overlap significantly, with the endpoints for each QTL corresponding to positions 30,542,688 and 36,339,580 of the B73 genome or 30,685,578 and 36,015,815 of the B73 genome. Without wishing to be bound by theory, the K22 QTL and the CIMBL83 and CML304 QTL are believed to comprise the same causative gene. SEQ ID NOs: 25-33 disclose the sequences for genes that are believed to be causative gene for each inbred line's corresponding QTL.

### Genetic Mapping

Specific genetic loci correlating with a particular phenotype, such as disease resistance, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods are available for detecting molecular markers or clusters of molecular markers that co-segregate with a trait of interest, such as a disease resistance trait. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) population-based association analysis (i.e., association mapping) and 2) traditional linkage analysis.

### Association Mapping

Understanding the extent and patterns of linkage disequilibrium (LD) in the genome is a prerequisite for developing efficient association approaches to identify and map quantitative trait loci (QTL). LD refers to the non-random association of alleles in a collection of individuals. When LD is observed among alleles at linked loci, it is measured as LD decay across a specific region of a chromosome. The extent of the LD reflects the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al. (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Elite breeding lines provide a valuable starting point for association analyses. Association analyses use quantitative phenotypic scores (e.g., disease tolerance rated from one to nine for each line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable map (for example, a composite map) can optionally be used to help observe distribution of the identified QTL markers and/or QTL marker clustering using previously determined map locations of the markers.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Marker loci that demonstrate statistically significant co-segregation with a disease resistance trait, as determined by traditional linkage analysis and by whole genome association analysis, are provided herein. Detection of these loci or additional linked loci can be used in marker assisted breeding programs to produce plants comprising disease resistance.

Activities in marker assisted breeding programs may include but are not limited to: selecting among new breeding populations to identify which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences.

### Chromosomal Intervals

Chromosomal intervals that correlate with the disease resistance trait are provided. A variety of methods are available for identifying chromosomal intervals. The boundaries of such chromosomal intervals are drawn to encompass markers that will be linked to the gene(s) controlling the trait of interest. In other words, the chromosomal interval is drawn such that any marker that lies within that interval (including the terminal markers that define the boundaries of the interval) can be used as a marker for a disease resistance trait.

Conversely, e.g., if two markers in close proximity show co-segregation with the desired phenotypic trait, it is sometimes unclear if each of those markers identify the same gene or two different gene or multiple genes. Regardless, knowledge of how many genes are in a particular physical/genomic interval is not necessary to make or practice certain examples of the present disclosure.

Thus, disclosed herein is an interval on maize chromosome 3. The disclosed interval on chromosome 3 can encompass 1, 2, 3, 4, 5, 6, 7, 8, 9, 10, 11, or 12 of the markers for SLB resistance disclosed in Table 1. In particular examples, the interval on chromosome 3 can encompass 1, 2, 3, 4, 5, 6, 7, 8, 9, 10, 11, or 12 of the markers D6, C10, A10, G6, H6, D10, F10, K6, L6, O6, Q6, and G3. In particular examples, the interval on chromosome 3 can encompass 1, 2, 3, 4, 5, 6, 7, 8, 9, 10, 11, or 12 of the markers D7, L4, E7, G7, H7, I7, J7, K7, L7, M7, N7, and O4. Any marker located within these intervals can find use as a marker for SLB resistance and can be used in the context of the methods presented herein to select plants that have resistance to SLB, compared to a control plant. In certain examples, markers located upstream and downstream of P450 gene position are closely linked genetically and physically and hence may be used to select the P450 gene for trait introgression and products development.

### Markers and Linkage Relationships

A common measure of linkage is the frequency with which traits cosegregate. This can be expressed as a percentage of cosegregation (recombination frequency) or in centiMorgans (CM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less such as, for example, about 9% or less, about 8% or less, about 7% or less, about 6% or less, about 5% or less, about 4% or less, about 3% or less, or about 2% or less. In some examples, the relevant loci (e.g., a marker locus and a target locus) display a recombination frequency of about 1% or less, e.g., about 0.75% or less, about 0.5% or less, or about 0.25% or less. Thus, the loci are about 10 cM, 9 cM, 8 CM, 7 CM, 6 CM, 5 CM, 4 CM, 3 CM, 2 CM, 1 cM, 0.75 CM, 0.5 CM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with a disease resistance trait, it is important to note that the marker locus is not necessarily responsible for the expression of the disease resistance phenotype. For example, it is not a requirement that the marker polynucleotide sequence be part of a gene that is responsible for the disease resistant phenotype (for example, is part of the gene open reading frame). The association between a specific marker allele and the disease resistance trait is due to the original “coupling” linkage phase between the marker allele and the allele in the ancestral line from which the allele originated. Eventually, with repeated recombination, crossing over events between the marker and genetic locus can change this orientation. For this reason, the favorable marker allele may change depending on the linkage phase that exists within the parent comprising resistance to the disease that is used to create segregating populations. This does not change the fact that the marker can be used to monitor segregation of the phenotype. It only changes which marker allele is considered favorable in a given segregating population.

Methods presented herein include detecting the presence of one or more marker alleles associated with disease resistance in a plant and then selecting plants that have favorable alleles at those marker loci. Markers have been identified herein as being associated with the disease resistance trait and hence can be used to predict disease resistance in a plant. Any marker within 50 cM, 40 cM, 30 CM, 20 cM, 15 cM, 10 cM, 9 CM, 8 CM, 7 CM, 6 CM, 5 CM, 4 CM, 3 CM, 2 cM, 1 CM, 0.75 cM, 0.5 CM or 0.25 CM (based on a single meiosis based genetic map) could also be used to predict disease resistance in a plant.

### Marker Assisted Selection

Molecular markers can be used in a variety of plant breeding applications (e.g., see Staub et al. (1996) Hortscience 31:729-741; Tanksley (1983) Plant Molecular Biology Reporter. 1:3-8). One area of interest is to increase the efficiency of backcrossing and introgressing genes using marker-assisted selection (MAS). A molecular marker that demonstrates linkage with a locus affecting a desired phenotypic trait provides a useful tool for the selection of the trait in a plant population. This is particularly true where the phenotype is hard to assay. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Use of flanking markers decreases the chances that false positive selection will occur as a double recombination event would be needed. In some cases, a marker is located within the gene itself, so that recombination cannot occur between the marker and the gene. In some examples, the methods disclosed herein produce a marker in a disease resistance gene, wherein the gene was identified by inferring genomic location from clustering of conserved domains or a clustering analysis.

When a gene is introgressed by MAS, it is not only the gene that is introduced but also the flanking regions (Gepts. (2002). Crop Sci; 42:1780-1790). This is referred to as “linkage drag.” In the case where the donor plant is highly unrelated to the recipient plant, these flanking regions carry additional genes that may code for agronomically undesirable traits. Linkage drag may also result in reduced yield or other negative agronomic characteristics even after multiple cycles of backcrossing into the elite line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). In classical breeding it is usually only by chance that recombinations are selected that contribute to a reduction in the size of the donor segment (Tanksley et al. (1989). Biotechnology 7:257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

Implementation of MAS can include: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols. SSRs can be defined as relatively short runs of tandemly repeated DNA with lengths of 6 bp or less (Tautz (1989) Nucleic Acid Research 17:6463-6471; Wang et al. (1994) Theoretical and Applied Genetics, 88:1-6). Polymorphisms arise due to variation in the number of repeat units, probably caused by slippage during DNA replication (Levinson and Gutman (1987) Mol Biol Evol 4:203-221). The variation in repeat length may be detected by designing PCR primers to the conserved non-repetitive flanking regions (Weber and May (1989) Am J Hum Genet. 44:388-396). SSRs are highly suited to mapping and MAS as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus potentially providing the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as SNPs do not require large amounts of DNA and automation of the assay may be straight-forward. SNPs also have the promise of being relatively low-cost systems. These three factors together make SNPs highly attractive for use in MAS. Several methods are available for SNP genotyping and/or detection, including but not limited to, hybridization, primer extension, oligonucleotide ligation, nuclease cleavage, minisequencing, and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele “T” for a specific line or variety with disease resistance, but the allele ‘T’ might also occur in the breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. Using automated high throughput marker detection platforms makes this process highly efficient and effective.

Many of the markers presented herein can readily be used as single nucleotide polymorphic (SNP) markers to select for the P450 gene. Using PCR, the primers are used to amplify DNA segments from individuals (e.g., an inbred) that represent the diversity in the population of interest. The PCR products are sequenced directly in one or both directions. The resulting sequences are aligned, and polymorphisms are identified. The polymorphisms are not limited to single nucleotide polymorphisms (SNPs), but also include indels, CAPS, SSRs, and VNTRs (variable number of tandem repeats). Specifically, with respect to the fine map information described herein, one can readily use the information provided herein to obtain additional polymorphic SNPs (and other markers) within the region amplified by the primers disclosed herein. Markers within the described map region can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the species, or even across other species that have been genetically or physically aligned.

In general, MAS uses polymorphic markers that have been identified as displaying a significant likelihood of co-segregation with a trait such as the SLB disease resistance trait and/or genes disclosed herein. Such markers are presumed to map near a gene or genes that give the plant its disease resistant phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with SLB disease resistance may be selected for by detecting one or more marker alleles, and in addition, progeny plants derived from those plants can also be selected. Hence, a plant containing a desired genotype in a given chromosomal region (i.e. a genotype associated with disease resistance) is obtained and then crossed to another plant. The progeny of such a cross would then be evaluated genotypically using one or more markers and the progeny plants with the same genotype in a given chromosomal region would then be selected as comprising disease resistance.

The SNPs could be used alone or in combination (i.e. a SNP haplotype) to select for a favorable resistant gene allele associated with GLS disease resistance. For example, a SNP haplotype can include a combination of 2, 3, 4, 5, 6, 7, 8, 9, 10, 11, or 12 of the markers for SLB resistance in Table 1 of this disclosure. The 2-12 markers can be (1) the K22 markers or (2) the CIMBL83/CML304 markers.

The skilled artisan would expect that there might be additional polymorphic sites at marker loci in and around a chromosome marker identified by the methods disclosed herein, wherein one or more polymorphic sites is in linkage disequilibrium (LD) with an allele at one or more of the polymorphic sites in the haplotype and thus could be used in a marker assisted selection program to introgress a gene allele or genomic fragment of interest. Two particular alleles at different polymorphic sites are said to be in LD if the presence of the allele at one of the sites tends to predict the presence of the allele at the other site on the same chromosome (Stevens, Mol. Diag. 4:309-17 (1999)). The marker loci can be located within 5 CM, 2 cM, or 1 cM (on a single meiosis based genetic map) of the disease resistance trait QTL.

Allelic frequency (and hence, haplotype frequency) can differ from one germplasm pool to another. Germplasm pools vary due to maturity differences, heterotic groupings, geographical distribution, etc. As a result, SNPs and other polymorphisms may not be informative in some germplasm pools.

### Methods of Selecting Plants Comprising Resistance to SLB

Provided herein is a method of identifying a maize plant comprising a marker allele associated with resistance to southern leaf blight. The method comprises screening nucleic acid from a maize plant for at least one of the marker alleles on chromosome 3 disclosed herein. The plant may be a member of a population of maize plants and nucleic acid may be isolated form a plurality of plants in order to screen for a plant comprising the at least one marker.

Marker alleles (e.g., single nucleotide polymorphisms (SNP)) are provided in Table 1. The right column of Table 1 provides each SNP and flanking genomic sequence for context. The residue associated with resistance to SLB is listed before the slash, and the residue associated with susceptibility to SLB is listed after the slash. The SNP is shown in bold typeface and within brackets. The sequence of only one strand is provided, but the disclosed SNPs and methods of using encompass all complementary forms (e.g., in the context of the reverse complement strand).

TABLE 1

| | | Genome | | |
|---|---|---|---|---|
| | | Position | | |
| | SNP Linked | (B73 | SEQ | |
| Marker | with | Version | ID | |
| Identifier | Resistance | 4.0) | NO | Sequence with SNP |
| D6 | C | 30,542,688 | 1 | accagaccggtccgatgcgccagctgtccgagagccaa |
| | | | | ttgcagaactct[c/t]tgcacacacggtccggtgtgccag |
| | | | | ctgatagcccacttcttgaatcttct |
| C10 | C | 31,720,421 | 2 | cacgtcgaccctgaccctgtacttgtaccgcttggcaaag |
| | | | | aagacgaaaa[c/t]gagcaggttgacggtgctgagcac |
| | | | | ggcgagcagccagtacaggtagtcga |
| A10 | C | 32,082,590 | 3 | acaccaaatttagtagcactagtgcgatgggacagtccg |
| | | | | gcttgttagcc[c/t]ggaccatccagaagtaaattagttcg |
| | | | | agcgagtcttgatttgctacgaga |
| G6 | G | 32,315,947 | 4 | accgatggttttctctacgcgcgcgctcccttcccctctacct |
| | | | | ctgacta[g/c]tgggaccggtctgtcggcaccagccgcc |
| | | | | ctcgctcgcgtgcccgctccct |
| H6 | G | 32,686,155 | 5 | aaacagccacattttgcatacatatcaagaagggcactg |
| | | | | cctgagaacac[g/a]ctggacatgtgccctgatgttgtga |
| | | | | ggtaagcatgcaattgccttcctat |
| D10 | T | 32,974,680 | 6 | aaaagaaattaaaaggaaaaaactaatccccacggca |
| | | | | acaagacaaccaa[t/c]cagttcaaccctgcaactgca |
| | | | | ggggtattattgtcgcaggctcgcctata |
| F10 | C | 33,064,162 | 7 | tgtctcagctatcgaaggagttaaagctattttgatgtgagg |
| | | | | tcttattt[c/a]tgaacttgtagtttgtcattaaagtttatatattg |
| | | | | tgatagtcgtagtgc |
| K6 | A | 33,094,714 | 8 | aatgatatctctagaaccaaatagctctaatggaatgatat |
| | | | | ctctagaag[a/c]aaatacttgtaaaagtctctttatatcctt |
| | | | | aagaataccatctagtaact |
| L6 | C | 33,507,499 | 9 | agaagtccagacatatatcatcactatagaggtgctcgtc |
| | | | | aagaggcatg[c/t]tgctcatgttaaacagttggtcaaaa |
| | | | | aagttcttattgtcgtccaaagta |
| O6 | T | 34,671,241 | 10 | cttcagtgccccctccttggtccctagcgcggcggcgcctg |
| | | | | caacaggga[t/c]ctctatggctccacgattactatcagct |
| | | | | tccggctgtgttgaaaaccatt |
| Q6 | G | 35,061,932 | 11 | aagtgattattaaagagtagcattttggattgagtcatgtgg |
| | | | | actaatca[g/t]agtgttagtattatgtaacacatctctttgct |
| | | | | tgtagtgcacatgtgtgg |
| G3 | C | 36,339,580 | 12 | aattgaaccagagaaggtatctaagaggagggtcttacc |
| | | | | ggagtatcgga[c/t]agcgcaggggcccggtcgtcgac |
| | | | | ggagggcgtcacactccgtatgtttgg |
| D7 | T | 30,685,578 | 13 | tatgaatggagtattacacttaggtatttagcacatcatcatc |
| | | | | caccgta[t/c]cttgttcccctgccaagttcctactgaggta |
| | | | | gactcataatcagtctttc |
| L4 | T | 30,979,851 | 14 | ctcccggagctcaagaactgaatgaaactattgctgctgt |
| | | | | agctttagct[t/a]gctaaatgcctgcatgctgcaccagct |
| | | | | gttgtatctcccagctctcgaag |
| E7 | T | 31,720,421 | 15 | cacgtcgaccctgaccctgtacttgtaccgcttggcaaag |
| | | | | aagacgaaaa[t/c]gagcaggttgacggtgctgagcac |
| | | | | ggcgagcagccagtacaggtagtcga |
| G7 | A | 31,844,546 | 16 | gaaaattcagtctcaaatacaggcagcacacctagtttcc |
| | | | | ctgggaacac[a/g]aaatcgaaaagagaccagagctc |
| | | | | agaaagtttattttgaataggtgcacc |
| H7 | G | 32,752,561 | 17 | cagtggaacatatactgcttgctcacaaaatgagttcttgct |
| | | | | tgcttaca[g/t]gctaatgatggtgacaaaaatcagatcta |
| | | | | aagaggagtaccttacaatca |
| I7 | T | 32,785,178 | 18 | ccatccgcgagatgaggaatagacgagacaagctcgg |
| | | | | agggatggctgtc[t/c]gacgtccaaaggcagggcgtct |
| | | | | ttgcgtctttccacgcggaagctaggct |
| J7 | T | 33,110,398 | 19 | ccttcttccccgtggaaggtgcagcaactgctgccgctgtg |
| | | | | cgcggtcct[t/c]gccgtcgtcgtcgcgtggtgtgccgcgc |
| | | | | gcgcggcggagtgggcgtggct |
| K7 | G | 33,138,036 | 20 | cagctgacgcggctgctgcactatggcgtgagcacccac |
| | | | | gagggcagcaa[g/a]tgggccaagcaccgcaggatca |
| | | | | tcaaccccgctttccatttcgagaagct |
| L7 | A | 34,202,712 | 21 | agcggcggcggaagtgcgccggcgggctcgtgggcgg |
| | | | | gagcgagaggatg[a/g]ggtgggacccaagctgcgtc |
| | | | | ggggaagggtagaggatcctggccctgggg |
| M7 | T | 34,263,189 | 22 | ggataggatgtgatattacacgctgtttctcgatgtagcggg |
| | | | | atgctcat[t/c]accctagttttgaggggtattatggtttagg |
| | | | | ggcgctcaagttggtcaga |
| N7 | A | 35,355,465 | 23 | gacattaatgttttcgctcagatgattgacgacgcggaagg |
| | | | | cctggaaaa[a/g]atcgagaacctacagagccatgata |
| | | | | ataatgaaatctatgaaaaggctgt |
| O4 | A | 36,015,815 | 24 | acgtattttacttggtacctccttagcttgatgtcaccttactat |
| | | | | atctg[a/t]aatcagaagcatagcctgtggtgcatggccg |
| | | | | gagctaagatactacaagg |

In some examples, the method comprises selecting a maize plant (e.g., from a population) that comprises the at least one marker. In some examples, selecting the maize plant comprises choosing a plant for further use in, for example breeding activities (e.g., crosses, backcrosses, or introgression). Selecting the plant that comprising the at least one marker can provide a plant that will be an appropriate donor of the SLB resistance trait during breeding activities.

In some examples, the method of selecting a maize plant comprising resistance to southern leaf blight comprises obtaining a nucleic acid sample from a plurality of plants, seeds, tissues, or germplasm in a population; screening each sample for the at least one marker allele located on chromosome 3; and selecting one or more of the plants, seeds, tissues, or germplasm comprising at least one of the marker alleles associated with resistance to SLB.

In some examples, the method of selecting a maize plant comprising resistance to southern leaf blight comprises crossing the selected plant with a second maize plant. The second maize plant may be from a population of plants used for backcrossing and/or introgression of the SLB resistance trait into a population of plants that lacks the trait. In some examples, the method comprises obtaining a progeny plant from the cross that comprises at least one marker allele set forth in Table 1.

In some examples, the method may further comprise isolating nucleic acid from the maize plant or maize plant population. The isolation of nucleic acid can facilitate the detection of the at least one marker. Nucleic acid isolation can comprise any method suitable to provide genomic material for PCR, sequencing procedures, and/or identification of the at least one marker.

In some examples, the screened for marker allele is located in a chromosomal interval comprising the QTL of CIMBL83 and CML304 or a portion of the interval that nonetheless comprises the causative gene (e.g., a portion reduced by recombination). In some examples, the screened for marker allele is located in a chromosomal interval flanked by and including the markers D6, corresponding to a “C” at position 51 of SEQ ID NO: 1 and G3, corresponding to a “C” at position 51 of SEQ ID NO: 12. The interval may also comprise one or more of markers C10, A10, G6, H6, D10, F10, K6, L6, O6, and Q6.

In some examples, the screened for marker allele is located in a chromosomal interval comprising the QTL of K22 or a portion of the interval that nonetheless comprises the causative gene (e.g., a portion reduced by recombination). In some examples, the screened for marker allele is located in a chromosomal interval flanked by and including the markers D7, corresponding to a “T” at position 51 of SEQ ID NO: 13 and O4, corresponding to an “A” at position 51 of SEQ ID NO: 24. The interval may also comprise one or more of markers L4, E7, G7, H7, I7, J7, K7, L7, M7, and N7.

In some examples, one or more of markers D6, C10, A10, G6, H6, D10, F10, K6, L6, O6, Q6, and G3 are detected. These markers were found to be linked to the SLB resistance QTL of CIMBL83 and CML304 and are also linked to each other. Thus, the presence and detection of one or more of these markers is linked to SLB resistance. In some examples, each of D6, C10, A10, G6, H6, D10, F10, K6, L6, O6, Q6, and G3 are detected. In some examples, one or more of markers G6, H6, D10, F10, K6, and L6 are detected. In some examples, markers G6 and L6 are detected. In some examples, markers K6 and L6 are detected.

In some examples, one or more of markers D7, L4, E7, G7, H7, I7, J7, K7, L7, M7, N7, and O4 are detected. These markers were found to be linked to the SLB resistance QTL of K22 and are also linked to each other. Thus, the presence and detection of one or more of these markers is linked to SLB resistance. In some examples, each of markers D7, L4, E7, G7, H7, I7, J7, K7, L7, M7, N7, and O4 are detected. In some examples, one or more of markers I7, J7, K7, and L7, are detected. In some examples, markers I7 and L7 are detected.

### Methods of Producing Maize Plants Comprising Resistance to SLB

Provided herein is a method of producing a maize plant comprising a QTL allele associated with resistance to SLB. The method comprises screening a maize plant population for a QTL allele associated with resistance to SLB. The QTL allele comprises one or more of the following marker alleles: (1) D6, C10, A10, G6, H6, D10, F10, K6, L6, O6, Q6, and G3 or one or more of (2) D7, L4, E7, G7, H7, I7, J7, K7, L7, M7, N7, and O4. The method comprises selecting from the population at least one maize plant comprising the QTL allele. The selection can be based on any result indicating that the QTL is present. For example, sequencing of the genomic region can be performed to determine whether one or more markers are present in a subject plant. The method comprises crossing the selected maize plant with a second maize plant and obtaining a progeny plant that comprises the QTL allele. The progeny plant can then be used for further breeding activities and/or as a source of seed.

In some examples, the method comprises introgressing the QTL allele into a second maize plant population. Introgression can be accomplished via repeated backcrossing and can be used to produce, e.g., a plant with one or more desired agronomic features as well as increased SLB resistance due to the presence of the QTL allele.

In some examples, the QTL allele is located in a chromosomal interval flanked by and including the markers D6, corresponding to a “C” at position 51 of SEQ ID NO: 1 and G3, corresponding to a “C” at position 51 of SEQ ID NO: 12.

In some examples, the QTL allele is located in a chromosomal interval flanked by and including the markers D7, corresponding to a “T” at position 51 of SEQ ID NO: 13 and O4, corresponding to an “A” at position 51 of SEQ ID NO: 24.

In some examples, one or more of markers D6, C10, A10, G6, H6, D10, F10, K6, L6, O6, Q6, and G3 are detected. These markers were found to be linked to the SLB resistance QTL of CIMBL83 and CML304 and are also linked to each other. Thus, the presence and detection of one or more of these markers is linked to SLB resistance. In some examples, each of markers D6, C10, A10, G6, H6, D10, F10, K6, L6, O6, Q6, and G3 are detected. In some examples, one or more of markers G6, H6, D10, F10, K6, and L6 are detected. In some examples, markers G6 and L6 are detected. In some examples, markers K6 and L6 are detected.

In some examples, one or more of markers D7, L4, E7, G7, H7, I7, J7, K7, L7, M7, N7, and O4 are detected. These markers were found to be linked to the SLB resistance QTL of K22 and are also linked to each other. Thus, the presence and detection of one or more of these markers is linked to SLB resistance. In some examples, each of markers D7, L4, E7, G7, H7, I7, J7, K7, L7, M7, N7, and O4 are detected. In some examples, one or more of markers I7, J7, K7, and L7, are detected. In some examples, markers I7 and L7 are detected.

Methods of Selecting and/or Producing Maize Plants Encoding SLB Resistance Genes

Provided herein are methods of selecting a maize plant comprising resistance to southern leaf blight. The method comprises screening a population of maize plants for the presence of a gene encoding a protein comprising at least 95% amino acid sequence identity to one or more of SEQ ID NOs: 27, 30, or 33. The method also comprises selecting a maize plant comprising the gene.

In some examples, the method comprises crossing the selected maize plant with a second maize plant. In some examples, the method comprises obtaining a progeny plant that comprises the gene. In some examples, progeny plants derived from the cross can then be used for further breeding activities and/or as a source of seed. In some examples, the gene can be introgressed into a plant breeding line (e.g., an elite line) via repeated backcrossing using the progeny plant(s).

### SLB Resistance Genes

In various aspects of the present disclosure, P450 genes that convey SLB resistance are used to produce recombinant plants or plants are screened for said genes. For example, these genes can be detected to determine if a given plant comprises resistance to SLB, transformed into a plant, or added into a plant genome using site-specific genome editing techniques (e.g., CRISPR-, TALENs-, meganuclease-, or zinc finger nuclease-based techniques). SLB resistance-conferring P450 gene sequences were identified in the inbred lines K22, CIMBL83, and CML304. Unless noted otherwise, any P450 gene sequence presented herein (e.g., any nucleic acid or protein sequence from SEQ ID NOs: 25-33) (See Table 2) can be used in any aspect of the present disclosure requiring a specific P450 gene/protein sequence. Additionally, the P450 gene sequences presented herein comprise protein sequences, DNA coding sequences (e.g., cDNA sequences), or genomic DNA sequences that include promoter, terminator, exon, and intron sequences. Accordingly, these sequences are described in more detail below.

In some examples, the protein encoded by the gene comprises at least 90% amino acid sequence identity to one or more of SEQ ID NOs: 27, 30, or 33. For example, the gene can comprise at least 95% amino acid sequence identity to one or more of SEQ ID NOs: 27, 30, or 33. In another example, the gene can comprise at least 96%, at least 97%, at least 98%, or at least 99% amino acid sequence identity to one or more of SEQ ID NOs: 27, 30, or 33. In another example, the gene can comprise at least 85% amino acid sequence identity to one or more of SEQ ID NOs: 27, 30, or 33. In another example, the gene can comprise 100% amino acid sequence identity to one or more of SEQ ID NOs: 27, 30, or 33.

In some examples, the gene is encoded by a specific nucleotide sequence. This sequence can include only coding sequence (e.g., a cDNA sequence), or both coding sequences and noncoding sequences (e.g., exons and promoters, terminators, and/or introns). In some examples, the promoter either naturally occurs with the gene or is heterologous to the gene. In some examples, the terminator either naturally occurs with the gene or is heterologous to the gene.

In some examples, the gene comprises a nucleotide sequence comprising at least 95% identity to one or more of SEQ ID NOs: 25, 26, 28, 29, 31, or 32. In some examples, the gene comprises a nucleotide sequence comprising at least 90% identity to one or more of SEQ ID NOs: 25, 26, 28, 29, 31, or 32. In some examples, the gene comprises a nucleotide sequence comprising at least 96% identity to one or more of SEQ ID NOs: 25, 26, 28, 29, 31, or 32. In some examples, the gene comprises a nucleotide sequence comprising at least 97% identity to one or more of SEQ ID NOs: 25, 26, 28, 29, 31, or 32. In some examples, the gene comprises a nucleotide sequence comprising at least 98% identity to one or more of SEQ ID NOs: 25, 26, 28, 29, 31, or 32. In some examples, the gene comprises a nucleotide sequence comprising at least 99% identity to one or more of SEQ ID NOs: 25, 26, 28, 29, 31, or 32. In some examples, the gene comprises a nucleotide sequence comprising 100% identity to one or more of SEQ ID NOs: 25, 26, 28, 29, 31, or 32.

TABLE 2

| SEQ | |
|---|---|
| ID | |
| NO | Sequence |
| 25 | gcgtttcgagactagggggtccctgggccgacgagtgaatgtcgccgcgtgccccagccc |
| | agatgggtcggcgcgaggccgagcgcgaaagggggaagtgaggtggccggagacgggcgt |
| | gagagaggtggaaatcccgcggccttcgtgttcgtcccgcgcccaggtcgggtgcgcttg |
| | cagtagggggttacaagcgtccacgcgggagagggagcgagcggcctcacgcgagcgcct |
| | gtctcgtcctcgtccccgcgcggccaaccctgtgtaagagggccctggtccttcctttta |
| | taggcgtaaggagaggatccaggtgtacaatgggggtgtagcagagtgctacgtgtctag |
| | cggaggagagctagcgccctaagtacatgccgtcgtggcagccggagagattttggcacc |
| | cggttcgtgtggtgtcgtggccgtcggaggagcgctggagcctggtggaaggacagctgt |
| | cggggctgtcgagtccttgctgacgtcatcttgcttccgtaagggggcggagagctgccg |
| | tcatcagggagcgtgcggggcgccatcattgcctatctggcggagcgagccagatgggac |
| | gccggtcttgttccccgtagcctgagtcagcttggggtagggtaatgatgacgcctcctg |
| | ttgacgtggtcggctcgcgcccgaggttgggcgatgtggaggctcctccgaggtcgaggt |
| | cgagtctgtcttccgtggccgtggtcgagtccgagcctctgggtcgggcgaggcggagac |
| | cgtcggctgaggtcagggctgagtccgagccctggggtcgggcgaagcggagttcgtcgt |
| | cttctggggctgagcccaagtccgagccctgggtcgggcggagcggagttcgtcgtcttc |
| | tggggcttagcccgagtccgagccctgggtcgggcggagcggagttcgccgtcttctagg |
| | gcttagcccgagtccgagccctgggtcgggcggagcggaattcgccatcttccggggctt |
| | agcccgagtccgagccctgggtcgggcggagcggagttcgccgtcttcaggggctgagcc |
| | cgagtccgagccctgggtcgggcgaagcggagcttcctatggtgcctgcggccgggcctg |
| | actgcctgtcagcctcactctgtcaagtggcaccgcagtcggagcggcgcaggcggcgct |
| | gtctttctgtcaggccggtcagtggagcggcgaagtgacggcggtcacttcggctctgtc |
| | gactgaagggcgcgcgtcaggataaaggtgtcaggccacctttgcattaaatgctcctgc |
| | gatttggttggtcggcgcggcgatttggtcagggttgcttcttggcgaagacagggcctc |
| | gggcgagccggaagtatgttcgccgctggaggggggcctcgggcgaaacggagatcctcc |
| | gggatcggctgcccttgtccgaggctaggctcgggcgaggcgtgatcgagtccctcgaat |
| | ggaccgatcccttacttaatcgcacccatcaggcctttgcagctttatgctgatggggtt |
| | accagttgagaattaggaaccttgagggtactcctaattatggtccccgacaccaggcga |
| | tggcttctgcaagaaatgtcgtggcttacggtgcaagctagtatatttacaaagggatat |
| | ttcgacatgcaaaaaaactcgtgccgtttaaaggacaactagaaatgcgacatttgaagt |
| | cagaatgcataatataattaaaaaatatatattttcgaaatgaatattatgttacacatg |
| | tttgactaaaaaataaattctaaataaattaaaaaaccattttgtgcatgatttatttga |
| | attagcgtgtaatgaaaaaacaaataaatattgttgctttagaagactgaatttaggttg |
| | ttggagaccgagaaaatatatatgacataattttttataatgtgttgtaaaggatgaaaa |
| | atatttatttagggatagaatttaggggctggagagtggagacggtctaagcgtatagga |
| | cgacggcgatggcgagcagctctctccgttcggttccccttccgctgcacacagaaaagt |
| | cacgagcttagatttttttcacccaagtggctggcggctagccaagcgcccaagtcatgg |
| | cgacgagcgtgccagagggattgctcggcgagccttcttccccgtggaaggtgcagcaac |
| | tgctgccgctgtgcgcggtcctcgccgtcgtcgtcgcgtggtgtgccgcgcgcgcggcgg |
| | agtgggcgtggctgaagccgcggcgcctggaacgggcgctgcgggcgcagggtatccgtg |
| | gcacggcgtaccgcccgctggccggcgacgcgccgctgtccgatcgactcgcccgcgagg |
| | ccaggtccaggccgcccctaccgccgggctgccacgccatcgtgcccagagccatgccgc |
| | tcgtccaccactccatgaacgagcacggtaacttctctctcgctgttgtcgcgttgacta |
| | cttgactctaaccggtgatctacctttagcttgccggtcgtaccacaccaactcactaag |
| | gaacgaacggtttttccatggtattacacaaacatttatcatagtaaatttcaaaatata |
| | aatcttatgtcatgaagttacataagttatttaataaaaatataaatatttgatttagaa |
| | taaagctaatacaaagtgtgctgtgggaaatatggtttcaagtagtttgcttgacatcga |
| | ggatcattgagttattggtactgattaagatatcaattagcgaataaatttcccctccag |
| | tgtggaatattaaaaaaaaaatagagtgcggaggattcaaccctgctcgcttaaggccct |
| | ttggcacggctccagctcgagcttatagtgaagcctagcaaatggtcaaaaaaatggctt |
| | ctggtcgatagtcctctaagagctggagtcgttcttttgttgggagcaaaagcataaaaa |
| | atatggcttctagcagctcctatatttttctcatacatcattcgaaaaacatatgagaaa |
| | ttattttgccaaacgaattgtaaaatgactttggctccttcagagaagccaaagagccag |
| | aggagtcagagccaaaactattttagaggaggcggagcgctggcaaagggccatagtata |
| | aagcacttggcttgtacaattgcaagtcgtgtttgtttatgtacatgttacgtaaagaag |
| | agatattgtaaatatatacatatagtaataatttgaaaaaaaaaatataatcatgtttga |
| | ataaatatctcaattaagagaggactactccattatatatagaaaccgtagcaataacta |
| | gttgccatatactttgctagaagttgcatgttattcactaccggatagatctgctttgcc |
| | gagtggcaaagcccaggaaactctcggtaaaatctttgccgagtgtgactctctgtaaag |
| | aagtctcggcgaactgtacatcggcaatggcttctttgctgagtacgttttgtcgggcac |
| | ttggcaaagaaaagttaatgtcacggcgacaagggacggtgatgcagcctttgtcgagtg |
| | tcctctgtgacactcggcaaagaagcgttctttaccgagtgcttgttggcctgacattcg |
| | gcaaagaaagctctagtgggcctctttgtcagtccctttgtcgagagctctggtaggcac |
| | tcggtaaagaagctccctttgtcgagtgcctgttggactgccactcagcaaagaaagctc |
| | cagtgggcctctttgccggttcctttgccgagtgtgctgggaggcactcggcaaagggtg |
| | cttatttaccgagtgctgcgaccatatcactcgacaaagaagctttatcggttcccaagt |
| | gtcttctttgtcgagtgctttggttatcgcactcggcaaagaacctctttgccgagtgta |
| | acactccggaaattgaccagaaaaaaccttttttatttgtttttattattccatccaaac |
| | aaacaaaggatatatatcattgacatcacatatatcacataatcaacacatgtatcacca |
| | acaccataatctcacaaatatcaccacatcaaacaagtttcacaaacataaatctcacca |
| | agtatctcacaacatcaagtattatcaacatcaataagtttaacaaacacaaatatcacc |
| | aacactcgcaaacataagtctcaatcatcactaagaggttatcaacaaggtgggcggatg |
| | gactgttttagcgaagggttgggcaatctgtgagggttatttgatgtcgccgattatccc |
| | tgcatagagaagagattgcatttgtgagaccagatgaatatatattatgtaggactaaaa |
| | atttgatactcacaggagtatggaactgagcaaggtcagctggagggaacaacggaggtg |
| | gtggagcaagaccctgtgcagcgccaaagctctgcatgtactggaacatctctgccatcc |
| | tctgctgattcgccaagcgaaccacccgctccatcctcatcctcgcctctatctccatgt |
| | gtccctccctcttcttctagctgggcctgtaatatctcaccccaatgttataataatgca |
| | aagagaaggtatataactcaatgaatgacaaggcatagaagcactaacctggagttccaa |
| | gatacaatgttgggaactatcttgccaaaaggtcgtatggctaggctcgcgctcatactc |
| | cttgctcacacctgagacagagtgggagtggaggatgagttgattgccccgtcagcaatc |
| | cagtaccgcccatgcctcttgcctcctccgaccctcatgaggacatctctatcgatgtcc |
| | ttagtcctcggattgtaatctagcccatggacctcctttgccatggcagtgtactcactg |
| | aggcggctacggacggtaggttgctataagcctcaggcttgtgctccaggttgtaggtga |
| | catcggacgtcgccttgcccttgtgggccatagcatacgtcgagaagattgagctaggct |
| | agccaccatgtgacgtcggctacgataaaaccaacgagatggttagaaatcatgcctaat |
| | caaaagttagactaaattaaagaaagtgcatatacatgcttctatgtatttgccaaggct |
| | acgactgccttgatggtgggagggaccttgcatcatcaaactccgttcctggctagcgtt |
| | gtgcgcctcgtcccactcggccgagcaccacctttgcaccatctgctccaagcactcagg |
| | atgcgcgatgcaccaataaggaataatcttcaaagaaaacaattacacaacatatcaaaa |
| | gataaaattaagcatatttaatatcaatattaatgttctgtatttacctgcaagtactgg |
| | ttcagagtcaatgacatggttcgggcgtccttgctcaccttctccctaaggatggagtcg |
| | tggtaagtgatgatggcctagatgcacgcctcgtactgcatgtccacgatgagcttctta |
| | tggctcgcggtagccaccacatccatcgtggcctcatttctagcctcgtagctaaagata |
| | tcctacgtacaaagacgatgtattcatacattatttttaagaatgtgcaataaatgcaac |
| | atattaaacaatatagtgcaaggaagacttacccacagctcttgcttcatccgctccgcc |
| | ttgttgttgaattatctatcatctcgatccactgcatcgggggcgacagtgtagtggtcg |
| | aaggaataaaccaagcccatcactccggcatactcaacaagtctaggaaagtgttccctg |
| | cacagaagcccgaggatgccattggggttgcggcgtgacccctgcattcttcaaaacctt |
| | tcaagatatgtctaagtgattaagaaaaattatcagtttcttttttaaatttgaacatat |
| | aatatgaaaaagtatcgacaacatctaaagttgcatacctttccccatcgggtcgaatga |
| | gcgggcacctgtcacgaagtatcggtcgctgaaggagactcgcgagacctcgcaagtaga |
| | cgtgcatcgaacatgtggtgccagacctagaggcgacccgctgagcgtcttcagtcctac |
| | tgtgcctcctcctccatcctacgggtgctcctcatcctcctctctctcctcgtcccaccg |
| | cccaccatctttgtctaaccacctgcaattaagagtaagcaattaagcctagataaaaaa |
| | tatgtatttagaaacatatgtgaaataaataaaatatagcattacattgattaaaataat |
| | cttcatatgtgttcggattagccagatcataagtctcatcatcattatcaaccatttcat |
| | agtcaacatcatatgaaggcacaagtttataattgtaattgccttcaagcatttctaagt |
| | cattctcattttgcacttcattttcctcctcatcaacaaccatttcaatatctaccatcc |
| | atttcgattgctttggttaaatctatctcaaatcgctcttctagctattttcttaaaaga |
| | actctctgtcatatgtctctgggtctaagttataatcttcatcgtttagaataggtaatt |
| | tatcgtgcagtgataccatatacacagcatctcaacccttaagatattcttttatttggc |
| | acacatatgggagataatatacttatgtggtctgttggttcacgatatagacatcgtctc |
| | ctggtaaggtggaatcctatcgaatttcgattagcccaagattagaatgtgtccatctcg |
| | tcacttcagcgtcaaaccaatgacatttcagtatgaactgaaggtggcgtaggattgtca |
| | taaggattttgagctgcctcttttgtccatcacccgattctacctctatgaacctagacg |
| | atttacacttcagacggtactttgcctctgtgtattttttcctaaataggacacaaccct |
| | tcagacaaacatgtgtctgctcatacatcatcttgagtgcacgaagaagtttctatgcct |
| | cgtacatgctctttggcagaatgtgatcctctagaagcaaactgccaataactatcaaca |
| | atccatcaaaggtgtctcgacttaggctgtaatgcgacttgaactccattatacatccaa |
| | tggcatccagttgagaaacttttgtcttgccatgaaggggtttctgtgccacgtcaaaca |
| | tgtcgtagaacgcctttgtggtcgcctctagctcgtcctcagtatgtgattcagcgaatt |
| | atgcctcgtgatagtcgtttaccgctaccttggcaccaacatcataaccctcgatgcgtt |
| | gtctcgccacctcctctctcgtacgatgcgcttcaccatggaagatccatcgagtatagt |
| | ctggcgtaaatccattcttccaaatatgttgtctcatggctttctttgtttttcttttct |
| | tgttgtcacatttgttgcacggacatggaactagactcacgcctttagcagcttcgccaa |
| | atgctcgttccaagaaagcatcagtctttgtaatccattcaggagtgacatcattccttg |
| | ttacgtggtccatgtacatccactcacgatccttaatactctaacatatataagcaagta |
| | tagtctaatataaaaatgtaatgcatatacactatgttcctaccttctaatgggtgagga |
| | taggtcctaatcctacccgcagttgcgtagatggggttagtttccatgctctactctgat |
| | ccaagacagaatttcggcagcacctctccgttgttctatggaaacacatcctagctagga |
| | gactgtgtatccagagaacaacagggaggtgatgttgaaactctatctcgaatcggagta |
| | gagcatgtaaactaatttcgtctacgtaaccgcgggctatccaaaaaatatggacaattc |
| | gaaacatatacacatttgtagatatgtaaagatccacatatctatatcttatctctttcg |
| | aacgggagacgcctaactgggttacgtgatctacgaccatgacacagaaatcaaggttat |
| | accttaggtggcggtggagtcaggctagcggggctgtggtgcgtcggtgcagtggcaacg |
| | tgacacggtgcaggtagacccacaacagctaggaagccctctgcaaaaaaggaaaacaag |
| | tctgtcaagacaaaatttcgacagcacctttcctacatggtgatgttttcaaaacctgca |
| | aataaaactaacagcacgatggctgatatccacacatatagatcaaggccttgataacca |
| | catatacatatttgatctcacatattcatgcatgtttgttctcacaattcacaatcacac |
| | aaaaaaatagttcactaataataaatttcaaactcatagttcaaaataacttaacacaac |
| | ttcacaagtcacaatttcacaataacaactaaaaataactaggaccaaacatcaattact |
| | agccaaacatcatacaatttctatgtgtgttgcaaatgacaaaaaagagtctttaacttt |
| | gcttggccggagcacaggagagggcgcagatctaaggtggcggtgtgttagccggagcgg |
| | gggaccaattgatggggatggtcggagccagcggggacgaggggccggagtcggtgtgga |
| | cggtggcgccctggtcgatggggacggtggcgcgctggcgctcgagtgggtgacggggcc |
| | gaagtcggcgtggacggtggcgctggccgacaaggacggtggtgcgctggcgctcgggcg |
| | ggcgacgcgagagcggtgaggtgctggcgtggcggcgagcgggtggggtgcatggcaacg |
| | gcaggcagcggttgagcgtgcgaaagaatgggaccacgggctttggtcattgttaagtaa |
| | acgttctttgccgagtgctcgcgacctcacactcggcaaagattttttttaaaatttaaa |
| | aatgttactttgccgagtgccatggatctgacactcggcatagtatactttaccaagtgt |
| | tctggatctaacactcggcaaagattattttgatttgaaaactactttgttgtgtgttcc |
| | tctgtagtatccaagacactcggcaaagtatatttttatttttatttttattcttttttc |
| | attaaactttttgtggtttgttcctatactatatagacctacatgttccattttagtata |
| | attatcaaagtgtttgctataaatattagatttagttcgtttaattgaattttttcggat |
| | aattcagatttaaacgaaaaatggaaaacagtgaatgcaaaaatgatttcatgttattta |
| | gcacaagttacggtctatttcataaacaaaccagaattttcgagcaccatgcttaaacat |
| | gaccatcaacctgtgatccagttattttaaaattgtataaaacacaaacaaaatcagcga |
| | atcatgaaacttgtcaacatgtcatgatatcatatatagagaatctgataaaattttaaa |
| | actatttcatgaaagttgttacgcactatacgtagaaaatctagctgaagttaatagaag |
| | ttttatgattttacgtgaagtccgactaattttgactcgtatgactcatgacccatccac |
| | tctggattgacaacgcgtggcctactggtctgggcactggccaggcaagacctcggtcac |
| | ctggttcgggccgacgccgagggtgatcataaccgagccggagctggtgcgccaggttct |
| | gtccaacaagttcggccacttcgagaaggccggcttcgggcagctgacgcggctgctgca |
| | ctatggcgtgagcacccacgagggcagcaaatgggccaagcaccgcaggatcatcaaccc |
| | cgctttccatttcgagaagctcaaggtctcgtctcatgcgtgaatcacctcatcaatagc |
| | ttgctagttgatggatctgatacgagcttcccgatcgaccgattctcctgcagcgcatgc |
| | tgccggcctttgcgtcgtgctgcgccgacctggtgagcacatgggagcgtctggtggtcg |
| | cggccgacggcgaaccatgcgaggtggacatctggccggagatgcagaggctgacggggg |
| | acgtcatctcccgcgtcgccttcggcagcagctacctggaaggcaggaggatcttcgagc |
| | tccaggaggagcaggtgcacctcgccatgctcgtcgccggcaagatacacatccctggat |
| | acatgtcagctcgtgctctctagctagctctgatcaaactacattctatattaatttgat |
| | tgtcagtgagtaactgacacgacaaaaagattttgatatacagaattgcaattttcgtgc |
| | gtgtaggatgctgcctacaagggtaaaccggcggatgaagcggatcgcggcggagatcga |
| | agggatccttagacggatgatcgtgacgagggagagctcgctgagggcaggaaaggcgac |
| | gagtgacgaccttctcggcctgctgctagagtccaacatggagcagctcaggggccaagg |
| | cgaaggcgaaggcggaggcggcgggacgagcacgagctccggcggcttgtccaccgacga |
| | catcattggagagtgcaagctgttctacttcgccggcatggagaccacgtcggtgctgct |
| | cacgtggactatgctggcgctctgcatgcacccggagtggcaggaccttgccagggagga |
| | ggtgctccgcgtcttcggcggcgcctcctgcagagtgccggactacgacggcctcagccg |
| | actgaaaatcgtaaccttcatttcgatagcaggatcagtcttgatcgattcgtccagtca |
| | gtgtcgtgtgaaatgtggtcgtaattgtgattcgcaatcgcgtctgcaggtgaccatggt |
| | gctgtacgaggtgctgcggctctacacgccgctgccggcgttgcaccggcggacatacaa |
| | gcccatggagcttgggggcgtcaggtacccggcgggcgtgatgctgatgctgccgctgct |
| | ctgcatccaccacgacaaggacgcgtgggggccggacgccgacgagttcaggccggagag |
| | gttcgcggagggagtcgccagagcgtcgtccaccggggacgcgccgccgtcgttcttccc |
| | gttcggctggggacctcggacctgcgtcggccagaccttcgcgctgctggaggccaagat |
| | agggctcgccatgattctggggagcttcacgttcgagctctccccgtcctactcgcacgc |
| | gccgttccccgtcgtcttgctcaagccagagcacggcgcgcaggtcaagctcaggaagct |
| | ccaatgcatgagcagcatccgcatgcacgctgcacagccgaaacggtcataaatttccct |
| | gtatatatatgcgaaaaaaggcaacaacatgtatggatggaccaggactattgttagtta |
| | atgcctaaaaatactctatgcgtcctaaaataagtatccgtatgtttaaggtgaatagat |
| | caatgtctttaagaaattatatgtccacctataaatatgtaactattgtacttcggaaaa |
| | atagaaggtagaaaagaacaactagagttagtttatcttgtaaatcgaggtgcacttatt |
| | ttgtcacagattttaaattctaggagtacacttattttgggatgtacaaagtagtcggct |
| | aaaacttgtcaactagttggtagtacataattgtttggattgtcaactaattattagacc |
| | ttttgtgaacataaggttgatatgtgctcttgtgttttggttgtgatgtatgattatcaa |
| | caagagcagtgtctttgggttaaatcgtgtggactaatcggatgtgagagtatttgtgca |
| | atatatctcttgtcttgtggtgtgtaggtttgaagtcttgagatggtagtcgacggcaag |
| | ggtgaaagtcatgcggaattgcgtcgatggactgggagtagtaaagaacgtacactgggt |
| | cttgactgagggactagagaagctagaagactagttatggcggttgacacctgtggacac |
| | tgataggtgtaaggatatggaggagtggatcgtatcattgacgtggttgtggaccgacgg |
| | aacacatgtccaagacgtgggatcacgtatgtagtacgctaactatgggggtttaggtga |
| | ttaagcctcaaaaccacccaacacaagttcgcgggtttcatcggatttttctcaaaacta |
| | gatggcatggttccgaagggaataggaagcgacatgtggcatcatcatgaatggtgtgcc |
| | aaggcaaagcaatttcgtgaagagtgtatgataatcatatcaaaattccaagagctagtc |
| | cattttgcccctatatctagtggtagtttaggatagaggaataaccctttataaataggt |
| | aggggctgaaccattcaaccatctctctagcacctctgtaatttgtattctttatttcct |
| | tctcattttgttaggagtccttagatgctttgaactttaatatccaagttcatgcaattt |
| | tgaggaaatgtggccataaccacactttgccttttgggttttcaattcgtgtcaaattta |
| | ataggttttttctagttattgaggttttgagttgatcttggtcggtactgacccaaacaa |
| | ccgatcgaccgaccacatgctacgccatattatcaaggatgtgtgacatggtttgaagcc |
| | gatttagggtctatttggttcaattgtagattcttcaaaatctaattctagccgtgagct |
| | ataaaaatattaaaatgagctaacgattataagaaaaataaaatatgtttggttgaaata |
| | actgc |
| 26 | atggcgacgagcgtgccagagggattgctcggcgagccttcttccccgtggaaggtgcag |
| | caactgctgccgctgtgcgcggtcctcgccgtcgtcgtcgcgtggtgtgccgcgcgcgcg |
| | gcggagtgggcgtggctgaagccgcggcgcctggaacgggcgctgcgggcgcagggtatc |
| | cgtggcacggcgtaccgcccgctggccggcgacgcgccgctgtccgatcgactcgcccgc |
| | gaggccaggtccaggccgcccctaccgccgggctgccacgccatcgtgcccagagccatg |
| | ccgctcgtccaccactccatgaacgagcacggcaagacctcggtcacctggttcgggccg |
| | acgccgagggtgatcataaccgagccggagctggtgcgccaggttctgtccaacaagttc |
| | ggccacttcgagaaggccggcttcgggcagctgacgcggctgctgcactatggcgtgagc |
| | acccacgagggcagcaaatgggccaagcaccgcaggatcatcaaccccgctttccatttc |
| | gagaagctcaagcgcatgctgccggcctttgcgtcgtgctgcgccgacctggtgagcaca |
| | tgggagcgtctggtggtcgcggccgacggcgaaccatgcgaggtggacatctggccggag |
| | atgcagaggctgacgggggacgtcatctcccgcgtcgccttcggcagcagctacctggaa |
| | ggcaggaggatcttcgagctccaggaggagcaggtgcacctcgccatgctcgtcgccggc |
| | aagatacacatccctggatacatgatgctgcctacaagggtaaaccggcggatgaagcgg |
| | atcgcggcggagatcgaagggatccttagacggatgatcgtgacgagggagagctcgctg |
| | agggcaggaaaggcgacgagtgacgaccttctcggcctgctgctagagtccaacatggag |
| | cagctcaggggccaaggcgaaggcgaaggcggaggcggcgggacgagcacgagctccggc |
| | ggcttgtccaccgacgacatcattggagagtgcaagctgttctacttcgccggcatggag |
| | accacgtcggtgctgctcacgtggactatgctggcgctctgcatgcacccggagtggcag |
| | gaccttgccagggaggaggtgctccgcgtcttcggcggcgcctcctgcagagtgccggac |
| | tacgacggcctcagccgactgaaaatcgtgaccatggtgctgtacgaggtgctgcggctc |
| | tacacgccgctgccggcgttgcaccggcggacatacaagcccatggagcttgggggcgtc |
| | aggtacccggcgggcgtgatgctgatgctgccgctgctctgcatccaccacgacaaggac |
| | gcgtgggggccggacgccgacgagttcaggccggagaggttcgcggagggagtcgccaga |
| | gcgtcgtccaccggggacgcgccgccgtcgttcttcccgttcggctggggacctcggacc |
| | tgcgtcggccagaccttcgcgctgctggaggccaagatagggctcgccatgattctgggg |
| | agcttcacgttcgagctctccccgtcctactcgcacgcgccgttccccgtcgtcttgctc |
| | aagccagagcacggcgcgcaggtcaagctcaggaagctccaatgcatgagcagcatccgc |
| | atgcacgctgcacagccgaaacggtcataa |
| 27 | matsvpegllgepsspwkvqqllplcavlavvvawcaaraaewawlkprrleralraqgi |
| | rgtayrplagdaplsdrlarearsrpplppgchaivpramplvhhsmnehgktsvtwfgp |
| | tprviitepelvrqvlsnkfghfekagfgqltrllhygvsthegskwakhrriinpafhf |
| | eklkrmlpafasccadlvstwerlvvaadgepcevdiwpemqrltgdvisrvafgssyle |
| | grrifelqeeqvhlamlvagkihipgymmlptrvnrrmkriaaeiegilrrmivtressl |
| | ragkatsddllgillesnmeqlrgqgegegggggtstssgglstddiigecklfyfagme |
| | ttsvlltwtmlalcmhpewqdlareevlrvfggascrvpdydglsrlkivtmvlyevlrl |
| | ytplpalhrrtykpmelggvrypagvmlmlpllcihhdkdawgpdadefrperfaegvar |
| | asstgdappsffpfgwgprtcvgqtfalleakiglamilgsftfelspsyshapfpvvll |
| | kpehgaqvklrklqcmssirmhaaqpkrs |
| 28 | aagccaggtgtcggcgtttcgagactggggggtccctgggccgacgagtgaatgtcgccg |
| | cgtgccccagcccagatgggtcggcgcgaggccgagcgcgaaagggggaagtgaggtggc |
| | cggagacgggcgtgagagaggtggaaatcccgcggccttcgtgttcgtcccgcgcccagg |
| | tcgggtgcgcttgcagtagggggttacaagcgtccacgcgggagagggagcgagcggcct |
| | cacgcgagcgcctgtctcgtcctcgtccccgcgcggccaaccctgtgtaagagggccctg |
| | gtccttccttttataggcgtaaggagaggatccaggtgtacaatgggggtgtagcagagt |
| | gctacgtgtctagcggaggagagctagcgccctaagtacatgccgtcgtggcagccggag |
| | agattttggcacccggttcgtgtggtgtcgtggccgtcggaggagcgctggagcctggtg |
| | gaaggacagctgtcggggctgtcgagtccttgctgacgtcatcttgcttccgtaaggggg |
| | cggagagctgccgtcatcagggagcgtgcggggcgccatcattgcctatctggcggagcg |
| | agccagatgggacgccggtcttgttccccgtagcctgagtcagcttggggtagggtaatg |
| | atgacgcctcctgttgacgtggtcggctcgcgcccgaggttgggcgatgtggaggctcct |
| | ccgaggttgaggtcgagtctgtcttccgtggccgtggtcgagtccgagcctctgggtcgg |
| | gcgaggcggagaccgtcggctgaggtcagggctgagtccgagccctggggtcgggcgaag |
| | cggagttcgtcgtcttctggggctgagcccaagtccgagccctgggtcgggcggagcgga |
| | gttcgtcgtcttctggggcttagcccgagtccgagccctgggtcgggcggagcggagttc |
| | gccgtcttctagggcttagcccgagtccgagccctgggtcgggcggagcggaattcgcca |
| | tcttccggggcttagcctgagtccgagccctgggtcgggcggagcggagttcgccgtctt |
| | caggggctgagcccgagtccgagccctgggtcgggcgaagcggagcttcctatggtgcct |
| | gcggccgggcctgactgcctgtcagcctcactctgtcaagtggcaccgcagtcggagcgg |
| | cgcaggcggcgctgtctttctgtcaggccggtcagtggagcggcgaagtgacggcggtca |
| | cttcggctctgtcgactgaagggcgcgcgtcaggataaaggtgtcaggccacctttgcat |
| | taaatgctcctgcgatttggttggtcggcgcggcgatttggtcagggttgcttcttggcg |
| | aagacagggcctcgggcgagccggaagtatgttcgccgctggaggggggcctcgggcgaa |
| | acggagatcctccggggtcggctgcccttgtccgaggctaggctcgggcgaggcgtgatc |
| | gagtccctcgaatggaccgatcccttacttaatcgcacccatcaggcctttgcagcttta |
| | tgctgatggggttaccagttgagaattaggaaccttgagggtactcctaattatggtccc |
| | cgacaccaggcgatggcttctgcaagaaatgtcgtggcttacggtgcaagctagtatatt |
| | tacaaagggatatttcgacatgcaaaaaaactcgtgccgtttaaaggacaactagaaatg |
| | cgacatttgaagtcagaatgcataatataattaaaaaatatatattttcgaaatgaatat |
| | tatgttacacatgtttgactaaaaaataaattctaaataaattaaaaaaccattttgtgc |
| | atgatttatttgaattagcgtgtaatgaaaaaacaaataaatattgttgctttagaagac |
| | tgaatttaggttgttggagaccgagaaaatatatatgacataattttttataatgtgttg |
| | taaaggatgaaaaatatttatttagggatagaatttaggggctggagagtggagacggtc |
| | taagcgtataggacgacggcgatggcgagcagctctctccgttcggttccccttccgctg |
| | cacacagaaaagtcacgagcttagatttttttcacccaagtggctggcggctagccaagc |
| | gcccaagtcatggcgacgagcgtgccagagggattgctcggcgagccttcttccccgtgg |
| | aaggtgcagcaactgctgccgctgtgcgcggtcctcgccgtcgtcgtcgcgtggtgtgcc |
| | gcgcgcgcggcggagtgggcgtggctgaagccgcggcgcctggaacgggcgctgcgggcg |
| | cagggtatccgtggcacggcgtaccgcccgctggccggcgacgcgccgctgtccgatcga |
| | ctcgcccgcgaggccaggtccaggccgcccctaccgccgggctgccacgccatcgtgccc |
| | agagccatgccgctcgtccaccactccatgaacgagcacggtaacttctctctcgctgtt |
| | gtcgcgttgactacttgactctaaccggtgatctacctttagcttgccggtcgtaccaca |
| | ccaactcactaaggaacgaacggtttttccatggtattacacaaacatttatcatagtaa |
| | atttcaaaatataaatcttatgtcatgaagttacataagttatttaataaaaatataaat |
| | atttgatttagaataaagctaatacaaagtgtgctgtgggaaatatggtttcaagtagtt |
| | tgcttgacatcgaggatcattgagttattggtactgattaagatatcaattagcgaataa |
| | atttcccctccagtgtggaatattaaaaaaaaaatagagtgcggaggattcaaccctgct |
| | cgcttaaggccctttggcacggctccagctcgagcttatagtgaagcctagcaaatggtc |
| | aaaaaaatggcttctggtcgatagtcctctaagagctggagtcgttcttttgttgggagc |
| | aaaagcataaaaaatatggcttctagcagctcctatatttttctcatacatcattcgaaa |
| | aacatatgagaaattattttgccaaacgaattgtaaaatgactttggctccttcagagaa |
| | gccaaagagccagaggagtcagagccaaaactattttagaggaggcggagccctggcaaa |
| | gggccatagtataaagcacttggcttgtacaattgcaagtcgtgtttgtttatgtacatg |
| | ttacgtaaagaagagatattgtaaatatatacatatagtaataatttgaaaaaaaaaata |
| | taatcatgtttgaataaatatctcaattaagagaggactactccattatatatagaaacc |
| | gtagcaataactagttgccatatactttgctagaagttgcatgttattcactaccggata |
| | gatctgctttgccgagtggcaaagcccaggaaactctcggtaaaatctttgccgagtgtg |
| | actctctgtaaagaagtctcggcgaactgtacatcggcaatggcttctttgctgagtacg |
| | ttttgtcgggcacttggcaaagaaaagttaatgtcacggcgacaagggacggtgatgcag |
| | cctttgtcgagtgtcctctgtgacactcggcaaagaagcgttctttaccgagtgcttgtt |
| | ggcctgacattcggcaaagaaagctctagtgggcctctttgtcagtccctttgtcgagag |
| | ctctggtaggcactcggtaaagaagctccctttgtcgagtgcctgttggactgccactca |
| | gcaaagaaagctccagtgggcctctttgccggttcctttgccgagtgtgctgggaggcac |
| | tcggcaaagggtgcttatttaccgagtgctgcgaccatatcactcgacaaagaagcttta |
| | tcggttcccaagtgtcttctttgtcgagtgctttggttatcgcactcggcaaagaacctc |
| | tttgccgagtgtaacactccggaaattgaccagaaaaaaccttttttatttgtttttatt |
| | attccatccaaacaaacaaaggatatatatcattgacatcacatatatcacataatcaac |
| | acatgtatcaccaacaccataatctcacaaatatcaccacatcaaacaagtttcacaaac |
| | ataaatctcaccaagtatctcacaacatcaagtattatcaacatcaataagtttaacaaa |
| | cacaaatatcaccaacactcgcaaacataagtctcaatcatcactaagaggttatcaaca |
| | aggtgggcggatggactgttttagcgaagggttgggcaatctgtgagggttatttgatgt |
| | cgccgattatccctgcatagagaagagattgcatttgtgagaccagatgaatatatatta |
| | tgtaggactaaaaatttgatactcacaggagtatggaactgagcaaggtcagctggaggg |
| | aacaacggaggtggtggagcaagaccctgtgcagcgccaaagctctgcatgtactggaac |
| | atctctgccatcctctgctgattcgccaagcgaaccacccgctccatcctcatcctcgcc |
| | tctatctccatgtgtccctccctcttcttctagctgggcctgtaatatctcaccccaatg |
| | ttataataatgcaaagagaaggtatataactcaatgaatgacaaggcatagaagcactaa |
| | cctggagttccaagatacaatgttgggaactatcttgccaaaaggtcgtatggctaggct |
| | cgcgctcatactccttgctcacacctgagacagagtgggagtggaggatgagttgattgc |
| | cccgtcagcaatccagtaccgcccatgcctcttgcctcctccgaccctcatgaggacatc |
| | tctatcgatgtccttagtcctcggattgtaatctagcccatggacctcctttgccatggc |
| | agtgtactcactgaggcggctacggacggtaggttgctataagcctcaggcttgtgctcc |
| | aggttgtaggtgacatcggacgtcgccttgcccttgtgggccatagcatacgtcgagaag |
| | attgagctaggctagccaccatgtgacgtcggctacgataaaaccaacgagatggttaga |
| | aatcatgcctaatcaaaagttagactaaattaaagaaagtgcatatacatgcttctatgt |
| | atttgccaaggctacgactgccttgatggtgggagggaccttgcatcatcaaactccgtt |
| | cctggctagcgttgtgcgcctcgtcccactcggccgagcaccacctttgcaccatctgct |
| | ccaagcactcaggatgcgcgatgcaccaataaggaataatcttcaaagaaaacaattaca |
| | caacatatcaaaagataaaattaagcatatttaatatcaatattaatgttctgtatttac |
| | ctgcaagtactggttcagagtcaatgacatggttcgggcgtccttgctcaccttctccct |
| | aaggatggagtcgtggtaagtgatgatggcctggatgcacgcctcgtactgcatgtccac |
| | gatgagcttcttatggctcgcggtagccaccacatccatcgtggcctcatttctagcctc |
| | gtagctaaagatatcctacgtacaaagacgatgtattcatacattatttttaagaatgtg |
| | caataaatgcaacatattaaacaatatagtgcaaggaagacttacccacagctcttgctt |
| | catccgctccgccttgttgttgaattatctatcatctcgatccactgcatcgggggcgac |
| | agtgtagtggtcgaaggaataaaccaagcccatcactccggcatactcaacaagtctagg |
| | aaagtgttccctgcacagaagcccgaggatgccattggggttgcggcgtgacccctgcat |
| | tcttcaaaacctttcaagatatgtctaagtgattaagaaaaattatcagtttctttttta |
| | aatttgaacatataatatgaaaaagtatcgacaacatctaaagttgcatacctttcccca |
| | tcgggtcgaatgagcgggcacctgtcacgaagtatcggtcgctgaaggagactcgcgaga |
| | cctcgcaagtagacgtgcatcgaacatgtggtgccagacctagaggcgacccgctgagcg |
| | tcttcagtcctactgtgcctcctcctccatcctacgggtgctcctcatcctcctctctct |
| | cctcgtcccaccgcccaccatctttgtctaaccacctgcaattaagagtaagcaattaag |
| | cctagataaaaaatatgtatttagaaacatatgtgaaataaataaaatatagcattacat |
| | tgattaaaataatcttcatatgtgttcggattagccagatcataagtctcatcatcatta |
| | tcaaccatttcatagtcaacatcatatgaaggcacaagtttataattgtaattgccttca |
| | agcatttctaagtcattctcattttgcacttcattttcctcctcatcaacaaccatttca |
| | atatctaccatccatttcgattgctttggttaaatctatctcaaatcgctcttctagcta |
| | ttttcttaaaagaactctctgtcatatgtctctgggtctaagttataatcttcatcgttt |
| | agaataggtaatttatcgtgcagtgataccatatacacagcatctcaacccttaagatat |
| | tcttttatttggcacacatatgggagataatatacttatgtggtctgttggttcacgata |
| | tagacatcgtctcctggtaaggtggaatcctatcgaatttcgattagcccaagattagaa |
| | tgtgtccatctcgtcacttcagcgtcaaaccaatgacatttcagtatgaactgaaggtgg |
| | cgtaggattgtcacaaggattttgagctgcctcttttgtccatcacccgattctacctct |
| | atgaacctagacgatttacacttcagacggtactttgcctctgtgtattttttcctaaat |
| | aggacacaacccttcagacaaacatgtgtctgctcatacatcatcttgagtgcacgaaga |
| | agtttctatgcctcgtacatgctctttggcagaatgtgatcctctagaagcaaactgcca |
| | ataactatcaacaatccatcaaaggtgtctcgacttaggctgtaatgcgacttgaactcc |
| | attatacatccaatggcatccagttgagaaacttttgtcttgccatgaaggggtttctgt |
| | gccacgtcaaacatgtcgtagaacgcctttgtggtcgcctctagctcgtcctcagtatgt |
| | gattcagcgaattatgcctcgtgatagtcgtttaccgctaccttggcaccaacatcataa |
| | ccctcgatgcgttgtctcgccacctcctctctcgtacgatgcgcttcaccatggaagatc |
| | catcgagtatagtctggcgtaaatccattcttccaaatatgttgtctcatggctttcttt |
| | gtttttcttttcttgttgtcacatttgttgcacggacatggaactagactcacgccttta |
| | gcagcttcgccaaatgctcgttccaagaaagcatcagtctttgtaatccattcaggagtg |
| | acatcattccttgttacgtggtccatgtacatccactcacgatccttaatactctaacat |
| | atataagcaagtatagtctaatataaaaatgtaatgcatatacactatgttcctaccttc |
| | taatgggtgaggataggtcctaatcctacccgcagttgcgtagatggggttagtttccat |
| | gctctactctgatccaagacagaatttcggcagcacctctccgttgttctatggaaacac |
| | atcctagctaggagactgtgtatccagagaacaacagggaggtgatgttgaaactctatc |
| | tcgaatcggagtagagcatgtaaactaatttcgtctacgtaaccgcgggctatccaaaaa |
| | atatggacaattcgaaacatatacacatttgtagatatgtaaagatccacatatctatat |
| | cttatctctttcgaacgggagacgcctaactgggttacgtgatctacgaccatgacacag |
| | aaatcaaggttataccttaggtggcggtggagtcaggctagcggggctgtggtgcgtcgg |
| | tgcagtggcaacgtgacacggtgcaggtagacccacaacagctaggaagccctctgcaaa |
| | aaaggaaaacaagtctgtcaagacaaaatttcgacagcacctttcctacatggtgatgtt |
| | ttcaaaacctgcaaataaaactaacagcacgatggctgatatccacacatatagatcaag |
| | gccttgataaccacatatacatatttgatctcacatattcatgcatgtttgttctcacaa |
| | ttcacaatcacacaaaaaaatagttcactaataataaatttcaaactcatagttcaaaat |
| | aacttaacacaacttcacaagtcacaatttcacaataacaactaaaaataactaggacca |
| | aacatcaattactagccaaacatcatacaatttctatgtgtgttgcaaatgacaaaaaag |
| | agtctttaactttgcttggccggagcacaggagagggcgcagatctaaggtggcggtgtg |
| | ttagccggagcgggggaccaattgatggggatggtcggagccagcggggacgaggggccg |
| | gagtcggtgtggacggtggcgccctggtcgatggggacggtggcgcgctggcgctcgagt |
| | gggtgacggggccgaagtcggcgtggacggtggcgctggccgacaaggacggtggtgcgc |
| | tggcgctcgggcgggcgacgcgagagcggtgaggtgctggcgtggcggcgagcgggtggg |
| | gtgcatggcaacggcaggcagcggttgagcgtgcgaaagaatgggaccacgggctttggt |
| | cattgttaagtaaacgttctttgccgagtgctcgcgacctcacactcggcaaagattttt |
| | ttaaaaatttaaaaatgttactttgccgagtgtcatggatctgacactcggcatagtata |
| | ctttaccaagtgttctggatctaacactcggcaaagattattttgatttgaaaactactt |
| | tgttgtgtgttcctctgtagtatccaagacactcggcaaagtatatttttatttttattt |
| | ttattcttttttcattaaactttttgtggtttgttcctatactatatagacctacatgtt |
| | ccattttagtataattatcaaagtgtttgctataaatattagatttagttcgtttaattg |
| | aattttttcggataattcagatttaaacgaaaaatggaaaacagtgaatgcaaaaatgat |
| | ttcatgttatttagcacaagttacggtctatttcataaacaaaccagaattttcgagcac |
| | catgcttaaacatgaccatcaacctgtgatccagttattttaaaattgtataaaacacaa |
| | acaaaatcagcgaatcatgaaacttgtcaacatgtcatgatatcatatatagagaatctg |
| | ataaaattttaaaactatttcatgaaagttgttacgcactatacgtagaaaatctagctg |
| | aagttaatagaagttttatgattttacgtgaagtccgactaattttgactcgtatgactc |
| | atgacccatccactctggattgacaacgcgtggcctactggtctgggcactggccaggca |
| | agacctcggtcacctggttcgggccgacgccgagggtgatcataaccgagccggagctgg |
| | tgcgccaggttctgtccaacaagttcggccacttcgagaaggccggcttcgggcagctga |
| | cgcggctgctgcactatggcgtgagcacccacgagggcagcaaatgggccaagcaccgca |
| | ggatcatcaaccccgctttccatttcgagaagctcaaggtctcgtctcatgcgtgaatca |
| | cctcatcaatagcttgctagttgatggatctgatacgagcttcccgatcgaccgattctc |
| | ctgcagcgcatgctgccggcctttgcgtcgtgctgcgccgacctggtgagcacatgggag |
| | cgtctggtggtcgcggccgacggcgaaccatgcgaggtggacatctggccggagatgcag |
| | aggctgacgggggacgtcatctcccgcgtcgccttcggcagcagctacctggaaggcagg |
| | aggatcttcgagctccaggaggagcaggtgcacctcgccatgctcgtcgccggcaagata |
| | cacatccctggatacatgtcagctcgtgctctctagctagctctgatcaaactacattct |
| | atattaatttgattgtcagtgagtaactgacacgacaaaaagattttgatatacagaatt |
| | gcaattttcgtgcgtgtaggatgctgcctacaagggtaaaccggcggatgaagcggatcg |
| | cggcggagatcgaagggatccttagacggatgatcgtgacgagggagagctcgctgaggg |
| | caggaaaggcgacgagtgacgaccttctcggcctgctgctagagtccaacatggagcagc |
| | tcaggggccaaggcgaaggcgaaggcggaggcggcgggacgagcacgagctccggcggct |
| | tgtccaccgacgacatcattggagagtgcaagctgttctacttcgccggcatggagacca |
| | cgtcggtgctgctcacgtggactatgctggcgctctgcatgcacccggagtggcaggacc |
| | ttgccagggaggaggtgctccgcgtcttcggcggcgcctcctgcagagtgccggactacg |
| | acggcctcagccgactgaaaatcgtaaccttcatttcgatagcaggatcagtcttgatcg |
| | attcgtccagtcagtgtcgtgtgaaatgtggtcgtaattgtgattcgcaatcgcgtctgc |
| | aggtgaccatggtgctgtacgaggtgctgcggctctacacgccgctgccggcgttgcacc |
| | ggcggacatacaagcccatggagcttgggggcgtcaggtacccggcgggcgtgatgctga |
| | tgctgccgctgctctgcatccaccacgacaaggacgcgtgggggccggacgccgacgagt |
| | tcaggccggagaggttcgcggagggagtcgccagagcgtcgtccaccggggacgcgccgc |
| | cgtcgttcttcccgttcggctggggacctcggacctgcgtcggccagaccttcgcgctgc |
| | tggaggccaagatagggctcgccatgattctggggagcttcacgttcgagctctccccgt |
| | cctactcgcacgcgccgttccccgtcgtcttgctcaagccagagcacggcgcgcaggtca |
| | agctcaggaagctccaatgcatgagcagcatccgcatgcacgctgcacagccgaaacggt |
| | cataaatttccctgtatatatatgcgaaaaaaggcaacaacatgtatggatggaccagga |
| | ctattgttagttaatgcctaaaaatactctatgcgtcctaaaataagtatccgtatgttt |
| | aaggtgaatagatcaatgtctttaagaaattatatgtccacctataaatatgtaactatt |
| | gtacttcggaaaaatagaaggtagaaaagaacaactagagttagtttatcttgtaaatcg |
| | aggtgcacttattttgtcacagattttaaattctaggagtacacttattttgggatgtac |
| | aaagtagtcggctaaaacttgtcaactagttggtagtacataattgtttggattgtcaac |
| | taattattagaccttttgtgaacataaggttgatatgtgctcttgtgttttggttgtgat |
| | gtatgattatcaacaagagcagtgtctttgggttaaatcgtgtggactaatcggatgtga |
| | gagtatttgtgcaatatatctcttgtcttgtggtgtgtaggtttgaagtcttgagatggt |
| | agtcgacggcaagggtgaaagtcatgcggaattgcgtcgatggactgggagtagtaaaga |
| | acgtacactgggtcttgactgagggactagagaagctagaagactagttatggcggttga |
| | cacctgtggacactgataggtgtaaggatatggaggagtggatcgtatcattgacgtggt |
| | tgtggaccgacggaacacatgtccaagacgtgggatcacgtatgtagtacgctaactatg |
| | ggggtttaggtgattaagcctcaaaaccacccaacacaagttcgcgggtttcatcggatt |
| | tttctcaaaactagatggcatggttccgaagggaataggaagcgacatgtggcatcatca |
| | tgaatggtgtgccaaggcaaagcaatttcgtgaagagtgtatgataatcatatcaaaatt |
| | ccaagagctagtccattttgcccctatatctagtggtagtttaggatagaggaataaccc |
| | tttataaataggtaggggctgaaccattcaaccatctctctagcacctctgtaatttgta |
| | ttctttatttccttctcattttgttaggagtccttagatgctttgaactttaatatccaa |
| | gttcatgcaattttgaggaaatgtggccataaccacactttgccttttgggttttcaatt |
| | cgtgtcaaatttaataggttttttctagttattgaggttttgagttgatcttggtcggta |
| | ctgacccaaacaaccgatcgaccgaccacatgctacgccatattatcaaggatg |
| 29 | atggcgacgagcgtgccagagggattgctcggcgagccttcttccccgtggaaggtgcag |
| | caactgctgccgctgtgcgcggtcctcgccgtcgtcgtcgcgtggtgtgccgcgcgcgcg |
| | gcggagtgggcgtggctgaagccgcggcgcctggaacgggcgctgcgggcgcagggtatc |
| | cgtggcacggcgtaccgcccgctggccggcgacgcgccgctgtccgatcgactcgcccgc |
| | gaggccaggtccaggccgcccctaccgccgggctgccacgccatcgtgcccagagccatg |
| | ccgctcgtccaccactccatgaacgagcacggcaagacctcggtcacctggttcgggccg |
| | acgccgagggtgatcataaccgagccggagctggtgcgccaggttctgtccaacaagttc |
| | ggccacttcgagaaggccggcttcgggcagctgacgcggctgctgcactatggcgtgagc |
| | acccacgagggcagcaaatgggccaagcaccgcaggatcatcaaccccgctttccatttc |
| | gagaagctcaagcgcatgctgccggcctttgcgtcgtgctgcgccgacctggtgagcaca |
| | tgggagcgtctggtggtcgcggccgacggcgaaccatgcgaggtggacatctggccggag |
| | atgcagaggctgacgggggacgtcatctcccgcgtcgccttcggcagcagctacctggaa |
| | ggcaggaggatcttcgagctccaggaggagcaggtgcacctcgccatgctcgtcgccggc |
| | aagatacacatccctggatacatgatgctgcctacaagggtaaaccggcggatgaagcgg |
| | atcgcggcggagatcgaagggatccttagacggatgatcgtgacgagggagagctcgctg |
| | agggcaggaaaggcgacgagtgacgaccttctcggcctgctgctagagtccaacatggag |
| | cagctcaggggccaaggcgaaggcgaaggcggaggcgggggacgagcacgagctccggcg |
| | gcttgtccaccgacgacatcattggagagtgcaagctgttctacttcgccggcatggaga |
| | ccacgtcggtgctgctcacgtggactatgctggcgctctgcatgcacccggagtggcagg |
| | accttgccagggaggaggtgctccgcgtcttcggcggcgcctcctgcagagtgccggact |
| | acgacggcctcagccgactgaaaatcgtgaccatggtgctgtacgaggtgctgcggctct |
| | acacgccgctgccggcgttgcaccggcggacatacaagcccatggagcttgggggcgtca |
| | ggtacccggcgggcgtgatgctgatgctgccgctgctctgcatccaccacgacaaggacg |
| | cgtgggggccggacgccgacgagttcaggccggagaggttcgcggagggagtcgccagag |
| | cgtcgtccaccggggacgcgccgccgtcgttcttcccgttcggctggggacctcggacct |
| | gcgtcggccagaccttcgcgctgctggaggccaagatagggctcgccatgattctgggga |
| | gcttcacgttcgagctctccccgtcctactcgcacgcgccgttccccgtcgtcttgctca |
| | agccagagcacggcgcgcaggtcaagctcaggaagctccaatgcatgagcagcatccgca |
| | tgcacgctgcacagccgaaacggtcataa |
| 30 | matsvpegllgepsspwkvqqllplcavlavvvawcaaraaewawlkprrleralraqgi |
| | rgtayrplagdaplsdrlarearsrpplppgchaivpramplvhhsmnehgktsvtwfgp |
| | tprviitepelvrqvlsnkfghfekagfgqltrllhygvsthegskwakhrriinpafhf |
| | eklkrmlpafasccadlvstwerlvvaadgepcevdiwpemqrltgdvisrvafgssyle |
| | grrifelqeeqvhlamlvagkihipgymmlptrvnrrmkriaaeiegilrrmivtressl |
| | ragkatsddllglllesnmeqlrgqgegegggggtstssgglstddiigecklfyfagme |
| | ttsvlltwtmlalcmhpewqdlareevlrvfggascrvpdydglsrlkivtmvlyevlrl |
| | ytplpalhrrtykpmelggvrypagvmlmlpllcihhdkdawgpdadefrperfaegvar |
| | asstgdappsffpfgwgprtcvgqtfalleakiglamilgsftfelspsyshapfpvvll |
| | kpehgaqvklrklqcmssirmhaaqpkrs |
| 31 | gagactggggggtccctgggccgacgagtgaatgtcgccgcgtgccccagcccagatggg |
| | tcggcgcgaggccgagcgcgaaagggggaagtgaggtggccggagacgggcgtgagagag |
| | gtggaaatcccgcggccttcgtgttcgtcccgcgcccaggtcgggtgcgcttgcagtagg |
| | gggttacaagcgtccacgcgggagagggagcgagcggcctcacgcgagcgcctgtctcgt |
| | cctcgtccccgcgcggccaaccctgtgtaagagggccctggtccttccttttataggcgt |
| | aaggagaggatccaggtgtacaatgggggtgtagcagagtgctacgtgtctagcggagga |
| | gagctagcgccctaagtacatgccgtcgtggcagccggagagattttggcacccggttcg |
| | tgtggtgtcgtggccgtcggaggagcgctggagcctggtggaaggacagctgtcggggct |
| | gtcgagtccttgctgacgtcatcttgcttccgtaagggggcggagagctgccgtcatcag |
| | ggagcgtgcggggcgccatcattgcctatctggcggagcgagccagatgggacgccggtc |
| | ttgttccccgtagcctgagtcagcttggggtagggtaatgatgacgcctcctgttgacgt |
| | ggtcggctcgcgcccgaggttgggcgatgtggaggctcctccgaggtcgaggtcgagtct |
| | gtcttccgtggccgtggtcgagtccgagcctctgggtcgggcgaggcggagaccgtcggc |
| | tgaggtcagggctgagtccgagccctggggtcgggcgaagcggagttcgtcgtcttctgg |
| | ggctgagcccaagtccgagccctgggtcgggcggagcggagttcgtcgtcttctggggct |
| | tagcccgagtccgagccctgggtcgggcggagcggagttcgccgtcttctagggcttagc |
| | ccgagtccgagccctgggtcgggcggagcggaattcgccatcttccggggcttagcccga |
| | gtccgagccctgggtcgggcggagcggagttcgccgtcttcaggggctgagcccgagtcc |
| | gagccctgggtcgggcgaagcggagcttcctatggtgcctgcggccgggcctgactgcct |
| | gtcagcctcactctgtcaagtggcaccgcagtcggagcggcgcaggcggcgctgtctttc |
| | tgtcaggccggtcagtggagcggcgaagtgacggcggtcacttcggctctgtcgactgaa |
| | gggcgcgcgtcaggataaaggtgtcaggccacctttgcattaaatgctcctgcgatttgg |
| | ttggtcggcgcggcgatttggtcagggttgcttcttggcgaagacagggcctcgggcgag |
| | ccggaagtatgttcgccgctggaggggggcctcgggcgaaacggagatcctccggggtcg |
| | gctgcccttgtccgaggctaggctcgggcgaggcgtgatcgagtccctcgaatggaccga |
| | tcccttacttaatcgcacccatcaggcctttgcagctttatgctgatggggttaccagtt |
| | gagaattaggaaccttgagggtactcctaattatggtccccgacaccaggcgatggcttc |
| | tgcaagaaatgtcgtggcttacggtgcaagctagtatatttacaaagggatatttcgaca |
| | tgcaaaaaaactcgtgccgtttaaaggacaactagaaatgcgacatttgaagtcagaatg |
| | cataatataattaaaaaatatatattttcgaaatgaatattatgttacacatgtttgact |
| | aaaaaataaattctaaataaattaaaaaaccattttgtgcatgatttatttgaattagcg |
| | tgtaatgaaaaaacaaataaatattgttgctttagaagactgaatttaggttgttggaga |
| | ccgagaaaatatatatgacataattttttataatgtgttgtaaaggatgaaaaatattta |
| | tttagggatagaatttaggggctggagagtggagacggtctaagcgtataggacgacggc |
| | gatggcgagcagctctctccgttcggttccccttccgctgcacacagaaaagtcacgagc |
| | ttagatttttttcacccaagtggctggcggctagccaagcgcccaagtcatggcgacgag |
| | cgtgccagagggattgctcggcgagccttcttccccgtggaaggtgcagcaactgctgcc |
| | gctgtgcgcggtcctcgccgtcgtcgtcgcgtggtgtgccgcgcgcgcggcggagtgggc |
| | gtggctgaagccgcggcgcctggaacgggcgctgcgggcgcagggtatccgtggcacggc |
| | gtaccgcccgctggccggcgacgcgccgctgtccgatcgactcgcccgcgaggccaggtc |
| | caggccgcccctaccgccgggctgccacgccatcgtgcccagagccatgccgctcgtcca |
| | ccactccatgaacgagcacggtaacttctctctcgctgttgtcgcgttgactacttgact |
| | ctaaccggtgatctacctttagcttgccggtcgtaccacaccaactcactaaggaacgaa |
| | cggtttttccatggtattacacaaacatttatcatagtaaatttcaaaatataaatctta |
| | tgtcatgaagttacataagttatttaataaaaatataaatatttgatttagaataaagct |
| | aatacaaagtgtgctgtgggaaatatggtttcaagtagtttgcttgacatcgaggatcat |
| | tgagttattggtactgattaagatatcaattagcgaataaatttcccctccagtgtggaa |
| | tattaaaaaaaaaatagagtgcggaggattcaaccctgctcgcttaaggccctttggcac |
| | ggctccagctcgagcttatagtgaagcctagcaaatggtcaaaaaaatggcttctggtcg |
| | atagtcctctaagagctggagtcgttcttttgttgggagcaaaagtataaaaaatatggc |
| | ttctagcagctcctatatttttctcatacatcattcgaaaaacatatgagaaattatttt |
| | gccaaacgaattgtaaaatgactttggctccttcagagaagccaaagagccagaggagtc |
| | agagccaaaactattttagaggaggcggagccctggcaaagggccatagtataaagcact |
| | tggcttgtacaattgcaagtcgtgtttgtttatgtacatgttacgtaaagaagagatatt |
| | gtaaatatatacatatagtaataatttgaaaaaaaaaatataatcatgtttgaataaata |
| | tctcaattaagagaggactgctccattatatatagaaaccgtagcaataactagttgcca |
| | tatactttgctagaagttgcatgttattcactaccggatagatctgctttgccgagtggc |
| | aaagcccaggaaactctcggtaaaatctttgccgagtgtgactctctgtaaagaagtctc |
| | ggcgaactgtacatcggcaatggcttctttgctgagtacgttttgtcgggcacttggcaa |
| | agaaaagttaatgtcacggcgacaagggacggtgatgcagcctttgtcgagtgtcctctg |
| | tgacactcggcaaagaagcgttctttaccgagtgcttgttggcctgacattcggcaaaga |
| | aagctctagtgggcctctttgtcagtccctttgtcgagagctctggtaggcactcggtaa |
| | agaagctccctttgtcgagtgcctgttggactgccactcagcaaagaaagctccagtggg |
| | cctctttgccggttcctttgtcgagtgtgctgggaggcactcggcaaagggtgcttattt |
| | accgagtgctgcgaccatatcactcgacaaagaagctttatcggttcccaagtgtcttct |
| | ttgtcgagtgctttggttatcgcactcggcaaagaacctctttgccgagtgtaacactcc |
| | ggaaattgaccagaaaaaaccttttttatttgtttttattattccatccaaacaaacaaa |
| | ggatatatatcattgacatcacatatatcacataatcaacacatgtatcaccaacaccat |
| | aatctcacaaatatcaccacatcaaacaagtttcacaaacataaatctcaccaagtatct |
| | cacaacatcaagtattatcaacatcaataagtttaacaaacacaaatatcaccaacactc |
| | gcaaacataagtctcaatcatcactaagaggttatcaacaaggtgggcggatggactgtt |
| | ttagcgaagggttgggcaatctgtgagggttatttgatgtcgccgattatccctgcatag |
| | agaagagattgcatttgtgagaccagatgaatatatattatgtaggactaaaaatttgat |
| | actcacaggagtatggaactgagcaaggtcagctggagggaacaacggaggtggtggagc |
| | aagaccctgtgcagcgccaaagctctgcatgtactggaacatctctgccatcctctgctg |
| | attcgccaagcgaaccacccgctccatcctcatcctcgcctctatctccatgtgtccctc |
| | cctcttcttctagctgggcctgtaatatctcaccccaatgttataataatgcaaagagaa |
| | ggtatataactcaatgaatgacaaggcatagaagcactaacctggagttccaagatacaa |
| | tgttgggaactatcttgccaaaaggtcgtatggctaggctcgcgctcatactccttgctc |
| | acacctgagacagagtgggagtggaggatgagttgattgccccgtcagcaatccagtacc |
| | gcccatgcctcttgcctcctccgaccctcatgaggacatctctatcgatgtccttagtcc |
| | tcggattgtaatctagcccatggacctcctttgccatggcagtgtactcactgaggcggc |
| | tacggacggtaggttgctataagcctcaggcttgtgctccaggttgtaggtgacatcgga |
| | cgtcgccttgcccttgtgggccatagcatacgtcgagaagattgagctaggctagccacc |
| | atgtgacgtcggctacgataaaaccaacgagatggttagaaatcatgcctaatcaaaagt |
| | tagactaaattaaagaaagtgcatatacatgcttctatgtatttgccaaggctacgactg |
| | ccttgatggtgggagggaccttgcatcatcaaactccgttcctggctagcgttgtgcgcc |
| | tcgtcccactcggccgagcaccacctttgcaccatctgctccaagcactcaggatgcgcg |
| | atgcaccaataaggaataatcttcaaagaaaacaattacacaacatatcaaaagataaaa |
| | ttaagcatatttaatatcaatattaatgttctgtatttacctgcaagtactggttcagag |
| | tcaatgacatggttcgggcgtccttgctcaccttctccctaaggatggagtcgtggtaag |
| | tgatgatggcctggatgcacgcctcgtactgcatgtccacgatgagcttcttatggctcg |
| | cggtagccaccacatccatcgtggcctcatttctagcctcgtagctaaagatatcctacg |
| | tacaaagacgatgtattcatacattatttttaagaatgtgcaataaatgcaacatattaa |
| | acaatatagtgcaaggaagacttacccacagctcttgcttcatccgctccgccttgttgt |
| | tgaattatctatcatctcgatccactgcatcgggggcgacagtgtagtggtcgaaggaat |
| | aaaccaagcccatcactccggcatactcaacaagtctaggaaagtgttccctgcacagaa |
| | gcccgaggatgccattggggttgcggcgtgacccctgcattcttcaaaacctttcaagat |
| | atgtctaagtgattaagaaaaattatcagtttcttttttaaatttgaacatataatatga |
| | aaaagtatcgacaacatctaaagttgcatacctttccccatcgggtcgaatgagcgggca |
| | cctgtcacgaagtatcggtcgctgaaggagactcgcgagacctcgcaagtagacgtgcat |
| | cgaacatgtggtgccagacctagaggcgacccgctgagcgtcttcagtcctactgtgcct |
| | cctcctccatcctacgggtgctcctcatcctcctctctctcctcgtcccaccgcccacca |
| | tctttgtctaaccacctgcaattaagagtaagcaattaagcctagataaaaaatatgtat |
| | ttagaaacatatgtgaaataaataaaatatagcattacattgattaaaataatcttcata |
| | tgtgttcggattagccagatcataagtctcatcatcattatcaaccatttcatagtcaac |
| | atcatatgaaggcacaagtttataattgtaattgccttcaagcatttctaagtcattctc |
| | attttgcacttcattttcctcctcatcaacaaccatttcaatatctaccatccatttcga |
| | ttgctttggttaaatctatctcaaatcgctcttctagctattttcttaaaagaactctct |
| | gtcatatgtctctgggtctaagttataatcttcatcgtttagaataggtaatttatcgtg |
| | cagtgataccatatacacagcatctcaacccttaagatattcttttatttggcacacata |
| | tgggagataatatacttatgtggtctgttggttcacgatatagacatcgtctcctggtaa |
| | ggtggaatcctatcgaatttcgattagcccaagattagaatgtgtccatctcgtcacttc |
| | agcgtcaaaccaatgacatttcagtatgaactgaaggtggcgtaggattgtcacaaggat |
| | tttgagctgcctcttttgtccatcacccgattctacctctatgaacctagacgatttaca |
| | cttcagacggtactttgcctctgtgtattttttcctaaataggacacaacccttcagaca |
| | aacatgtgtctgctcatacatcatcttgagtgcacgaagaagtttctatgcctcgtacat |
| | gctctttggcagaatgtgatcctctagaagcaaactgccaataactatcaacaatccatc |
| | aaaggtgtctcgacttaggctgtaatgcgacttgaactccattatacatccaatggcatc |
| | cagttgagaaacttttgtcttgccatgaaggggtttctgtgccacgtcaaacatgtcgta |
| | gaacgcctttgtggtcgcctctagctcgtcctcagtatgtgattcagcgaattatgcctc |
| | gtgatagtcgtttaccgctaccttggcaccaacatcataaccctcgatgcgttgtctcgc |
| | cacctcctctctcgtacgatgcgcttcaccatggaagatccatcgagtatagtctggcgt |
| | aaatccattcttccaaatatgttgtctcatggctttctttgtttttcttttcttgttgtc |
| | acatttgttgcacggacatggaactagactcacgcctttagcagcttcgccaaatgctcg |
| | ttccaagaaagcatcagtctttgtaatccattcaggagtgacatcattccttgttacgtg |
| | gtccatgtacatccactcacgatccttaatactctaacatatataagcaagtatagtcta |
| | atataaaaatgtaatgcatatacactatgttcctaccttctaatgggtgaggataggtcc |
| | taatcctacccgcagttgcgtagatggggttagtttccatgctctactctgatccaagac |
| | agaatttcggcagcacctctccgttgttctatggaaacacatcctagctaggagactgtg |
| | tatccagagaacaacagggaggtgatgttgaaactctatctcgaatcggagtagagcatg |
| | taaactaatttcgtctacgtaaccgcgggctatccaaaaaatatggacaattcgaaacat |
| | atacacatttgtagatatgtaaagatccacatatctatatcttatctctttcgaacggga |
| | gacgcctaactgggttacgtgatctacgaccatgacacagaaatcaaggttataccttag |
| | gtggcggtggagtcaggctagcggggctgtggtgcgtcggtgcagtggcaacgtgacacg |
| | gtgcaggtagacccacaacagctaggaagccctctgcaaaaaaggaaaacaagtctgtca |
| | agacaaaatttcgacagcacctttcctacatggtgatgttttcaaaacctgcaaataaaa |
| | ctaacagcacgatggctgatatccacacatatagatcaaggccttgataaccacatatac |
| | atatttgatctcacatattcatgcatgtttgttctcacaattcacaatcacacaaaaaaa |
| | tagttcactaataataaatttcaaactcatagttcaaaataacttaacacaacttcacaa |
| | gtcacaatttcacaataacaactaaaaataactaggaccaaacatcaattactagccaaa |
| | catcatacaatttctatgtgtgttgcaaatgacaaaaaagagtctttaactttgcttggc |
| | cggagcacaggagagggcgcagatctaaggtggcggtgtgttagccggagcgggggacca |
| | atttatggggatggtcggagccagcggggacgaggggccggagtcggtgtggacggtggc |
| | gccctggtcgatggggacggtggcgcgctggcgctcgagtgggtgacggggccgaagtcg |
| | gcgtggacggtggcgctggccgacaaggacggtggtgcgctggcgctcgggcgggcgacg |
| | cgagagcggtgaggtgctggcgtggcggcgagcgggtggggtgcatggcaacggcaggca |
| | gcggttgagcgtgcgaaagaatgggaccacgggctttggtcattgttaagtaaacgttct |
| | ttgccgagtgctcgcgacctcacactcggcaaagattttttttaaaatttaaaaatgtta |
| | ctttgccgagtgccatggatctgacactcggcatagtatactttaccaagtgttctggat |
| | ctaacactcggcaaagattattttgatttgaaaactactttgttgtgtgttcctctgtag |
| | tatccaagacactcggcaaagtatatttttatttttatttttattcttttttcattaaac |
| | tttttgtggtttgttcctatactatatagacctacatgttccattttagtataattatca |
| | aagtgtttgctataaatattagatttagttcgtttaattgaattttttcggataattcag |
| | atttaaacgaaaaatggaaaacagtgaatgcaaaaatgatttcatgttatttagcacaag |
| | ttacggtctatttcataaacaaaccagaattttcgagcaccatgcttaaacatgaccatc |
| | aacctgtgatccagttattttaaaattgtataaaacacaaacaaaatcagcgaatcatga |
| | aacttgtcaacatgtcatgatatcatatatagagaatctgataaaattttaaaactattt |
| | catgaaagttgttacgcactatacgtagaaaatctagctgaagttaatagaagttttatg |
| | attttacgtgaagtccgactaattttgactcgtatgactcatgacccatccactctggat |
| | tgacaacgcgtggcctactggtctgggcactggccaggcaagacctcggtcacctggttc |
| | gggccgacgccgagggtgatcataaccgagccggagctggtgcgccaggttctgtccaac |
| | aagttcggccacttcgagaaggccggcttcgggcagctgacgcggctgctgcactatggc |
| | gtgagcacccacgagggcagcaaatgggccaagcaccgcaggatcatcaaccccgctttc |
| | catttcgagaagctcaaggtctcgtctcatgcgtgaatcacctcatcaatagcttgctag |
| | ttgatggatctgatacgagcttcccgatcgaccgattctcctgcagcgcatgctgccggc |
| | ctttgcgtcgtgctgcgccgacctggtgagcacatgggagcgtctggtggtcgcggccga |
| | cggcgaaccatgcgaggtggacatctggccggagatgcagaggctgacgggggacgtcat |
| | ctcccgcgtcgccttcggcagcagctacctggaaggcaggaggatcttcgagctccagga |
| | ggagcaggtgcacctcgccatgctcgtcgccggcaagatacacatccctggatacatgtc |
| | agctcgtgctctctagctagctctgatcaaactacattctatattaatttgattgtcagt |
| | gagtaactgacacgacaaaaatattttgatatacagaattgcaattttcgtgcgtgtagg |
| | atgctgcctacaagggtaaaccggcggatgaagcggatcgcggcggagatcgaagggatc |
| | cttagacggatgatcgtgacgagggagagctcgctgagggcaggaaaggcgacgagtgac |
| | gaccttctcggcctgctgctagagtccaacatggagcagctcaggggccaaggcgaaggc |
| | gaaggcggaggcggcgggacgagcacgagctccggcggcttgtccaccgacgacatcatt |
| | ggagagtgcaagctgttctacttcgccggcatggagaccacgtcggtgctgctcacgtgg |
| | actatgctggcgctctgcatgcacccggagtggcaggaccttgccagggaggaggtgctc |
| | cgcgtcttcggcggcgcctcctgcagagtgccggactacgacggcctcagccgactgaaa |
| | atcgtaaccttcatttcgatagcaggatcagtcttgatcgattcgtccagtcagtgtcgt |
| | gtgaaatgtggtcgtaattgtgattcgcaatcgcgtctgcaggtgaccatggtgctgtac |
| | gaggtgctgcggctctacacgccgctgccggcgttgcaccggcggacatacaagcccatg |
| | gagcttgggggcgtcaggtacccggcgggcgtgatgctgatgctgccgctgctctgcatc |
| | caccacgacaaggacgcgtgggggccggacgccgacgagttcaggccggagaggttcgcg |
| | gagggagtcgccagagcgtcgtccaccggggacgcgccgccgtcgttcttcccgttcggc |
| | tggggacctcggacctgcgtcggccagaccttcgcgctgctggaggccaagatagggctc |
| | gccatgattctggggagcttcacgttcgagctctccccgtcctactcgcacgcgccgttc |
| | cccgtcgtcttgctcaagccagagcacggcgcgcaggtcaagctcaggaagctcccatga |
| | gctgcatccgcttgcatattgcagagacgaaacggtcataaatttccctgtatatatacg |
| | gaaaaaaaaggcaacaacatgtttgctgtttggatggaccaggactattgttagtttatg |
| | cctaaaaatactctatatgcgtcctaaaataagtattcgtaggtttaaggtgaatagata |
| | attgtctttaagaaattacatgtccccctataaagatgtaactattgtactttggaaaag |
| | gagaaggtagaaaagaacaactagagttagtttaacttgtacctggaggtgcacttattt |
| | tgtcataatttttttaaattctaggagtacacttttttttgggatgtagagtagtccgct |
| | aaaatttactcaactagttgttagtccataattatttggattgtgagccaattattaggc |
| | attttgcaaacataaggtgtgtttcgattgtgacgagtaagtagtgtcttggattgagat |
| | gggtggactaattgggcatgagagtatttgtacaacacatctcttggcttgtagcgtgga |
| | gttgaaaatggtgatacgaagataaaagtcatgtggattcttgtcaatggatcgaaaacg |
| | gtaaggataagcactaggacttgatcgaacactaacatgtgcaaggatattagagcagat |
| | cataccaccgacacggttacgttacacactaacagaacacgtgtctaaaacatgggtcac |
| | gtatgaagttttcatacactacccacaagggtttcgatggtaaacctcaaaaccactcaa |
| | cactggttaaacctcaaaaccactcaacacgccagtccacgggttttgtctttaagaccc |
| | gatgacatagttccgaaggggtccaagcggcccctaacagaattgcgtaggaaaactcat |
| | ttttcgataatcacgtatatcacgactcaccgcgggtccaccggtaaatggcactcatca |
| | ggcccaaatcgaagcataatttgattggtgaactaggctctgcttgtctacgcacaaaac |
| | tgatgaggacatcaaaacttacaattcagtctaagtctagtgacatcatacctactacag |
| | tttatagaagtagaaaaggcaacagaaagaaatgtaaacctactgcagtttatggattct |
| | agctgctattcccaaataaattctcaaacaacttcggagccattggaggaagaaccatgg |
| | gctccggtgccatgaaattagtgatcttctcgtggacatggtacctgaaaaaatcacata |
| | acagaaatgagcataataccaaaaggggagttattggaaaattttcaaacgtcagcaaac |
| | cgcagaggatagagatcg |
| 32 | atggcgacgagcgtgccagagggattgctcggcgagccttcttccccgtggaaggtgcag |
| | caactgctgccgctgtgcgcggtcctcgccgtcgtcgtcgcgtggtgtgccgcgcgcgcg |
| | gcggagtgggcgtggctgaagccgcggcgcctggaacgggcgctgcgggcgcagggtatc |
| | cgtggcacggcgtaccgcccgctggccggcgacgcgccgctgtccgatcgactcgcccgc |
| | gaggccaggtccaggccgcccctaccgccgggctgccacgccatcgtgcccagagccatg |
| | ccgctcgtccaccactccatgaacgagcacggcaagacctcggtcacctggttcgggccg |
| | acgccgagggtgatcataaccgagccggagctggtgcgccaggttctgtccaacaagttc |
| | ggccacttcgagaaggccggcttcgggcagctgacgcggctgctgcactatggcgtgagc |
| | acccacgagggcagcaaatgggccaagcaccgcaggatcatcaaccccgctttccatttc |
| | gagaagctcaagcgcatgctgccggcctttgcgtcgtgctgcgccgacctggtgagcaca |
| | tgggagcgtctggtggtcgcggccgacggcgaaccatgcgaggtggacatctggccggag |
| | atgcagaggctgacgggggacgtcatctcccgcgtcgccttcggcagcagctacctggaa |
| | ggcaggaggatcttcgagctccaggaggagcaggtgcacctcgccatgctcgtcgccggc |
| | aagatacacatccctggatacatgatgctgcctacaagggtaaaccggcggatgaagcgg |
| | atcgcggcggagatcgaagggatccttagacggatgatcgtgacgagggagagctcgctg |
| | agggcaggaaaggcgacgagtgacgaccttctcggcctgctgctagagtccaacatggag |
| | cagctcaggggccaaggcgaaggcgaaggcggaggcggcgggacgagcacgagctccggc |
| | ggcttgtccaccgacgacatcattggagagtgcaagctgttctacttcgccggcatggag |
| | accacgtcggtgctgctcacgtggactatgctggcgctctgcatgcacccggagtggcag |
| | gaccttgccagggaggaggtgctccgcgtcttcggcggcgcctcctgcagagtgccggac |
| | tacgacggcctcagccgactgaaaatcgtgaccatggtgctgtacgaggtgctgcggctc |
| | tacacgccgctgccggcgttgcaccggcggacatacaagcccatggagcttgggggcgtc |
| | aggtacccggcgggcgtgatgctgatgctgccgctgctctgcatccaccacgacaaggac |
| | gcgtgggggccggacgccgacgagttcaggccggagaggttcgcggagggagtcgccaga |
| | gcgtcgtccaccggggacgcgccgccgtcgttcttcccgttcggctggggacctcggacc |
| | tgcgtcggccagaccttcgcgctgctggaggccaagatagggctcgccatgattctgggg |
| | agcttcacgttcgagctctccccgtcctactcgcacgcgccgttccccgtcgtcttgctc |
| | aagccagagcacggcgcgcaggtcaagctcaggaagctcccatga |
| 33 | matsvpegllgepsspwkvqqllplcavlavvvawcaaraaewawlkprrleralraqgi |
| | rgtayrplagdaplsdrlarearsrpplppgchaivpramplvhhsmnehgktsvtwfgp |
| | tprviitepelvrqvlsnkfghfekagfgqltrllhygvsthegskwakhrriinpafhf |
| | eklkrmlpafasccadlvstwerlvvaadgepcevdiwpemqrltgdvisrvafgssyle |
| | grrifelqeeqvhlamlvagkihipgymmlptrvnrrmkriaaeiegilrrmivtressl |
| | ragkatsddllgillesnmeqlrgqgegegggggtstssgglstddiigecklfyfagme |
| | ttsvlltwtmlalcmhpewqdlareevlrvfggascrvpdydglsrlkivtmvlyevlrl |
| | ytplpalhrrtykpmelggvrypagvmlmlpllcihhdkdawgpdadefrperfaegvar |
| | asstgdappsffpfgwgprtcvgqtfalleakiglamilgsftfelspsyshapfpvvll |
| | kpehgaqvklrklp |

Methods for Producing Recombinant Maize Plant Cells and/or Plants

Provided herein are methods for producing recombinant maize plants/cells. The recombinant plants comprise resistance to SLB. The methods comprise introducing a heterologous P450 gene sequence into the genome of a maize plant cell and selecting a plant cell comprising the heterologous P450 gene sequence encoded in the plant cell's genome.

In some examples, the method comprises introducing a site-specific modification of at least one target site into the genome of the maize plant cell and introducing a polynucleotide modification template comprising the heterologous P450 nucleotide sequence into the plant cell.

In some examples, the site specific modification is induced by a CRISPR-associated endonuclease.

In some examples, the site-specific modification comprises a single strand break or double strand break (DSB) that is made by a site-specific endonuclease such as, for example, TALENs, meganucleases, zinc finger nucleases, and CRISPR-associated (Cas) protein/guide polynucleotide complexes. In some examples, the introduction of a site-specific modification can be combined with the introduction of the polynucleotide modification template.

In some examples, a polynucleotide modification template is introduced into a cell by any method suitable to deliver the template into the nucleus of a cell, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

In some examples, the polynucleotide modification template may be introduced into a cell as a single stranded polynucleotide molecule, a double stranded polynucleotide molecule, or as part of a circular DNA (vector DNA). The polynucleotide modification template may also be tethered to the guide polynucleotide and/or the Cas endonuclease. Tethered templates can allow for co-localizing target and template DNA, useful in genome editing and targeted genome regulation, and can also be useful in targeting post-mitotic cells where function of endogenous homologous recombination HR machinery is expected to be highly diminished (Mali et al. 2013 Nature Methods Vol. 10:957-963.) The polynucleotide modification template may be present transiently in the cell or it can be introduced via a viral replicon.

The polynucleotide modification template, as used herein, refers to a polynucleotide that comprises at least one nucleotide modification when compared to the target nucleotide sequence to be edited. A nucleotide modification is at least one nucleotide substitution, addition or deletion. In some examples, the polynucleotide modification template can further comprise homologous nucleotide sequences flanking the at least one nucleotide modification, wherein the flanking homologous nucleotide sequences provide sufficient homology to support the incorporation of the polynucleotide modification template into the genome of the recipient plant cell.

The process for editing a genomic sequence combining site-specific modifications and modification templates generally comprises providing to a host cell a site-specific endonuclease, or a nucleic acid encoding a site-specific endonuclease, that recognizes a target sequence in the chromosomal sequence, and wherein the site-specific endonuclease induces a site-specific modification (e.g., a DSB) in the genomic sequence; and providing at least one polynucleotide modification template. The endonuclease may be provided to a cell by any method known in the art, for example, but not limited to transient introduction methods, transfection, microinjection, and/or topical application or indirectly via recombination constructs. The endonuclease may be provided as a protein or as a guided polynucleotide complex directly to a cell or indirectly via recombination constructs. The endonuclease may be introduced into a cell transiently or can be incorporated into the genome of the host cell. In the case of a CRISPR-Cas system, uptake of the endonuclease and/or the guided polynucleotide into the cell can be facilitated with a Cell Penetrating Peptide (CPP) as described in WO2016073433.

TAL effector nucleases (TALEN) are a class of sequence-specific nucleases that can be used to make double-strand breaks at specific target sequences in the genome of a plant or other organism. (See Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example comprising a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131, all of which are incorporated by reference herein.

The term “Cas gene” or “Cas protein” herein refers to a gene that is generally coupled, associated or close to, or in the vicinity of flanking CRISPR loci in bacterial systems. The terms “Cas” and “CRISPR-associated” are used interchangeably herein. The term “Cas endonuclease” herein refers to a protein, or complex of proteins, encoded by a Cas gene. A Cas endonuclease as disclosed herein, when in complex with a suitable polynucleotide component, is capable of recognizing, binding to, and optionally nicking or cleaving all or part of a specific DNA target sequence. A Cas endonuclease as described herein comprises one or more nuclease domains. Cas endonucleases of the disclosure includes those comprising a HNH or HNH-like nuclease domain and/or a RuvC or RuvC-like nuclease domain. A Cas endonuclease of the disclosure may include a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity but can still specifically bind to a DNA target sequence when complexed with a suitable RNA component. (See also U.S. Patent Application US 2015-0082478 A1, and US 2015-0059010 A1, both hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein, or a variant thereof, retaining some or all activity in each endonuclease domain of the Cas protein, is a suitable example of a Cas endonuclease that can cleave both strands of a DNA target sequence. A Cas9 protein comprising functional RuvC and HNH nuclease domains is an example of a Cas protein that can cleave both strands of a DNA target sequence. A guide polynucleotide/Cas endonuclease complex that can cleave one strand of a DNA target sequence can be characterized herein as comprising nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in U.S. Patent Appl. Publ. No. 2014/0189896, which is incorporated herein by reference.

In some examples, a pair of Cas9 nickases may be used to increase the specificity of DNA targeting. In general, this can be done by providing two Cas9 nickases that, by virtue of being associated with RNA components with different guide sequences, target and nick nearby DNA sequences on opposite strands in the region for desired targeting. Such nearby cleavage of each DNA strand creates a double strand break (i.e., a DSB with single-stranded overhangs), which is then recognized as a substrate for non-homologous-end-joining, NHEJ (prone to imperfect repair leading to mutations) or homologous recombination, HR. Each nick in these examples can be at least about 5, 10, 15, 20, 30, 40, 50, 60, 70, 80, 90, or 100 (or any integer between 5 and 100) bases apart from each other, for example. One or two Cas9 nickase proteins herein can be used in a Cas9 nickase pair. For example, a Cas9 nickase with a mutant RuvC domain, but functioning HNH domain (i.e., Cas9 HNH+/RuvC−), could be used (e.g., Streptococcus pyogenes Cas9 HNH+/RuvC−). Each Cas9 nickase (e.g., Cas9 HNH+/RuvC−) would be directed to specific DNA sites nearby each other (up to 100 base pairs apart) by using suitable RNA components herein with guide RNA sequences targeting each nickase to each specific DNA site.

In some examples, a Cas protein may be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains comprising one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16. See PCT patent applications PCT/US16/32073, filed May 12, 2016, and PCT/US16/32028 filed May 12, 2016 (both applications incorporated herein by reference) for more examples of Cas proteins.

A guide polynucleotide/Cas endonuclease complex in certain examples may bind to a DNA target site sequence but does not cleave any strand at the target site sequence. Such a complex may comprise a Cas protein in which all of its nuclease domains are mutant, dysfunctional. For example, a Cas9 protein herein that can bind to a DNA target site sequence but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein). In other aspects, an inactivated Cas protein may be fused with another protein comprising endonuclease activity, such as a FokI endonuclease.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and Ill are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

In some examples, the Cas endonuclease can comprise a modified form of the Cas9 polypeptide. The modified form of the Cas9 polypeptide can include an amino acid change (e.g., deletion, insertion, or substitution) that reduces the naturally-occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

In some examples, a catalytically inactive Cas9 can be fused to a heterologous sequence (US patent application US20140068797 A1). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double strand breaks (Guilinger et al. Nature Biotechnology, volume 32, number 6, June 2014).

In some examples, the method comprises growing a plant from the plant cell comprising the heterologous P450 gene sequence. In some examples, the plant exhibits increased resistance to southern leaf blight. In some examples, the plant derived from the method can then be used for further breeding activities and/or as a source of seed. In some examples, the P450 gene can be introgressed into a plant breeding line (e.g., an elite line) via repeated backcrossing using the plant produced by the method.

The heterologous P450 locus can be inserted into any location in the genome of the plant cell that is suitable for the expression of the heterologous P450 gene and, optionally, does not interfere with the expression of endogenous genes. In some examples, the heterologous P450 gene sequence replaces the endogenous P450 locus. In such examples, at least one allele of the endogenous P450 gene is eliminated and replaced by the heterologous P450 gene encoded on the polynucleotide modification template. In such examples, at least the coding regions of the endogenous P450 gene are replaced. Introns, promoter sequences, and terminator sequences may optionally be replaced as well.

In some examples, the heterologous P450 gene sequence is inserted into a locus different from the endogenous P450 locus. In some examples, the heterologous P450 gene sequence is inserted into a locus different from the endogenous P450 locus, along with other genes which are also inserted at the same locus, forming a molecular stack. In some examples, the molecular stack comprises a stack of disease resistance genes.

Also provided herein is a method for producing a recombinant maize plant cell. The method comprises introducing a P450 nucleotide sequence into one or more plant cells; inserting the P450 nucleotide sequence into the genome of at least one of the one or more plant cells; and selecting a plant cell that has inserted the P450 nucleotide sequence into the genome of the plant cell. The P450 nucleotide can comprise any P450 nucleotide sequence disclosed herein and can encode any of the P450 amino acid sequences disclosed herein.

In some examples, the method comprises growing a plant from the plant cell that has inserted the P450 nucleotide sequence into the genome of the plant cell. In some examples, the plant exhibits increased resistance to southern leaf blight. In some examples, the selection can be based on any result indicating that the P450 gene is present. For example, sequencing can be performed to determine if the gene has been inserted and/or plant cells can be selected for using a selectable marker. In some examples, the method comprises crossing the newly grown maize plant with a second maize plant and obtaining a progeny plant that comprises the P450 gene. The progeny plant can then be used for further breeding activities (e.g., backcrosses and/or introgression) and/or as a source of seed.

In some examples, introducing the P450 nucleotide sequence is accomplished via bacteria-mediated transformation.

In some examples, introducing the P450 nucleotide sequence is accomplished via biolistic transformation.

### Recombinant Plants and Seeds

Recombinant plants comprising the P450 gene sequences described herein are provided. Provided herein is a recombinant plant comprising a heterologous gene encoding a protein comprising at least 95% amino acid sequence identity to one or more of SEQ ID NOS: 27, 30, or 33. In some examples, the gene comprises a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.

Recombinant plant seeds comprising the P450 gene sequences described herein are provided. Provided herein is a recombinant plant seed comprising a heterologous gene encoding a protein comprising at least 95% amino acid sequence identity to one or more of SEQ ID NOs: 27, 30, or 33. In some examples, the gene comprises a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.

### Clauses of the Disclosure

Various aspects of the present disclosure will be understood by reference to the following clauses.
1. A method of identifying a maize plant comprising a marker allele associated with resistance to southern leaf blight, the method comprising:
   screening nucleic acid from a maize plant for at least one of the following marker alleles on chromosome 3:
      i. a “C” at D6, corresponding to position 51 of SEQ ID NO: 1;
      ii. a “C” at C10, corresponding to position 51 of SEQ ID NO: 2;
      iii. a “C” at A10, corresponding to position 51 of SEQ ID NO: 3;
      iv. a “G” at G6, corresponding to position 51 of SEQ ID NO: 4;
      v. a “G” at H6, corresponding to position 51 of SEQ ID NO: 5;
      vi. a “T” at D10, corresponding to position 51 of SEQ ID NO: 6;
      vii. a “C” at F10, corresponding to position 51 of SEQ ID NO: 7;
      viii. an “A” at K6, corresponding to position 51 of SEQ ID NO: 8;
      ix. a “C” at L6, corresponding to position 51 of SEQ ID NO: 9;
      x. a “T” at O6, corresponding to position 51 of SEQ ID NO: 10;
      xi. a “G” at Q6, corresponding to position 51 of SEQ ID NO: 11;
      xii. a “C” at G3, corresponding to position 51 of SEQ ID NO: 12;
      xiii. a “T” at D7, corresponding to position 51 of SEQ ID NO: 13; xiii.
      xiv. a “T” at L4, corresponding to position 51 of SEQ ID NO: 14;
      xv. a “T” at E7, corresponding to position 51 of SEQ ID NO: 15;
      xvi. an “A” at G7, corresponding to position 51 of SEQ ID NO: 16;
      xvii. a “G” at H7, corresponding to position 51 of SEQ ID NO: 17;
      xviii. a “T” at I7, corresponding to position 51 of SEQ ID NO: 18;
      xix. a “T” at J7, corresponding to position 51 of SEQ ID NO: 19;
      xx. a “G” at K7, corresponding to position 51 of SEQ ID NO: 20;
      xxi. an “A” at L7, corresponding to position 51 of SEQ ID NO: 21;
      xxii. a “T” at M7, corresponding to position 51 of SEQ ID NO: 22;
      xxiii. an “A” at N7, corresponding to position 51 of SEQ ID NO: 23; or
      xxiv. an “A” at O4, corresponding to position 51 of SEQ ID NO: 24,
   wherein the presence of the marker allele is associated with resistance to southern leaf blight.
2. The method of clause 1, further comprising
   a. obtaining a nucleic acid sample from a plurality of plants, seeds, tissues or germplasm in a population;
   b. screening each sample for at least one of the nucleic acid marker alleles on chromosome 3; and
   c. selecting one or more of the plants, seeds, tissues or germplasm comprising at least one of the marker alleles associated with resistance to southern leaf blight.
3. The method of clause 1 or 2, wherein the screened for marker allele is located in a chromosomal interval flanked by and including the markers D6, corresponding to a “C” at position 51 of SEQ ID NO: 1 and G3, corresponding to a “C” at position 51 of SEQ ID NO: 12.
4. The method of clause 1 or 2, wherein the screened for marker allele is located in a chromosomal interval flanked by and including the markers D7, corresponding to a “T” at position 51 of SEQ ID NO: 13 and O4, corresponding to an “A” at position 51 of SEQ ID NO: 24.
5. The method of any one of clauses 1-4, wherein one or more of markers i.-xii. are detected.
6. The method of any one of clauses 1-4, wherein one or more of markers xiii.-xxiv. are detected.
7. The method of any one of clauses 1-6, wherein each of markers i.-xii. are detected.
8. The method of any one of clauses 1-6, wherein each of markers xiii.-xxiv. are detected.
9. The method of any one of clauses 1-8, wherein one or more of markers iv.-ix. are detected, markers iv. and ix. are detected, or markers viii, and ix. are detected.
10. The method of any one of clauses 1-8, wherein one or more of markers xviii.-xxi. are detected or markers xviii, and xxi. are detected.
11. A method of producing a maize plant comprising a QTL associated with resistance to southern leaf blight, the method comprising:
   a. screening a maize plant population for a QTL associated with resistance to southern leaf blight, wherein the QTL comprises one or more marker alleles selected from the group consisting of:
      i. a “C” at D6, corresponding to position 51 of SEQ ID NO: 1;
      ii. a “C” at C10, corresponding to position 51 of SEQ ID NO: 2;
      iii. a “C” at A10, corresponding to position 51 of SEQ ID NO: 3;
      iv. a “G” at G6, corresponding to position 51 of SEQ ID NO: 4;
      v. a “G” at H6, corresponding to position 51 of SEQ ID NO: 5;
      vi. a “T” at D10, corresponding to position 51 of SEQ ID NO: 6;
      vii. a “C” at F10, corresponding to position 51 of SEQ ID NO: 7;
      viii. an “A” at K6, corresponding to position 51 of SEQ ID NO: 8;
      ix. a “C” at L6, corresponding to position 51 of SEQ ID NO: 9;
      x. a “T” at O6, corresponding to position 51 of SEQ ID NO: 10;
      xi. a “G” at Q6, corresponding to position 51 of SEQ ID NO: 11; and
      xii. a “C” at G3, corresponding to position 51 of SEQ ID NO: 12;
         or one or more marker alleles selected from the group consisting of:
      xiii. a “T” at D7, corresponding to position 51 of SEQ ID NO: 13;
      xiv. a “T” at L4, corresponding to position 51 of SEQ ID NO: 14;
      XV. a “T” at E7, corresponding to position 51 of SEQ ID NO: 15;
      xvi. an “A” at G7, corresponding to position 51 of SEQ ID NO: 16;
      xvii. a “G” at H7, corresponding to position 51 of SEQ ID NO: 17;
      xviii. a “T” at I7, corresponding to position 51 of SEQ ID NO: 18;
      xix. a “T” at J7, corresponding to position 51 of SEQ ID NO: 19;
      xx. a “G” at K7, corresponding to position 51 of SEQ ID NO: 20;
      xxi. an “A” at L7, corresponding to position 51 of SEQ ID NO: 21;
      xxii. a “T” at M7, corresponding to position 51 of SEQ ID NO: 22;
      xxiii. an “A” at N7, corresponding to position 51 of SEQ ID NO: 23; and
      xxiv. an “A” at O4, corresponding to position 51 of SEQ ID NO: 24;
   b. selecting from the population at least one maize plant comprising the QTL;
   C. crossing the maize plant selected in (b) with a second maize plant; and
   d. obtaining a progeny plant that comprises the QTL.
12. The method of clause 11, further comprising introgressing the QTL into a second maize plant population.
13. The method of clause 11 or 12, wherein the QTL is located in a chromosomal interval flanked by and including the markers D6, corresponding to a “C” at position 51 of SEQ ID NO: 1, and G3, corresponding to a “C” at position 51 of SEQ ID NO: 12.
14. The method of clause 11 or 12, wherein the QTL allele is located in a chromosomal interval flanked by and including the markers D7, corresponding to a “T” at position 51 of SEQ ID NO: 13 and O4, corresponding to an “A” at position 51 of SEQ ID NO: 24.
15. The method of any one of clauses 11-14, wherein the selected and progeny plants comprise one or more of markers i.-xii.
16. The method of any one of clauses 11-14, wherein the selected and progeny plants comprise one or more of markers xiii.-xxiv.
17. The method of any one of clauses 11-16, wherein the selected and progeny plants comprise each of markers i.-xii.
18. The method of any one of clauses 11-16, wherein the selected and progeny plants comprise each of markers xiii.-xxiv.
19. The method of any one of clauses 11-18, wherein the selected and progeny plants comprise one or more of markers iv.-ix., both of markers iv. and ix., or both of markers viii, and ix.
20. The method of any one of clauses 11-18, wherein the selected and progeny plants comprise one or more of markers xviii.-xxi, or both of markers xviii, and xxi.
21. A method of selecting a maize plant comprising resistance to southern leaf blight! comprising:
   a. screening a population of maize plants for the presence of a gene encoding a protein comprising at least 95% amino acid sequence identity to one or more of SEQ ID NOs: 27, 30, or 33 and
   b. selecting a maize plant comprising the gene.
22. The method of clause 21, wherein the protein comprises 100% amino acid sequence identity to SEQ ID NO: 27, 30, or 33.
23. The method of clause 21 or 22, further comprising:
   c. crossing the maize plant selected in (b) with a second maize plant; and
   d. obtaining a progeny plant that comprises the gene.
24. The method of any one of clauses 21-23, wherein the protein is encoded by a gene comprising a nucleotide sequence with at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.
25. The method of any one of clauses 21-24, wherein the protein is encoded by a gene comprising a nucleotide sequence comprising the sequence of SEQ ID NO: 25, SEQ ID NO: 26, SEQ ID NO: 28, SEQ ID NO: 29, SEQ ID NO: 31, or SEQ ID NO: 32.
26. A method for producing a recombinant maize plant cell, the method comprising:
   introducing a heterologous P450 gene sequence into the genome of a maize plant cell and
   selecting a plant cell comprising the heterologous P450 gene sequence encoded in the plant cell's genome.
27. The method of clause 26, further comprising:
   a) introducing a site-specific modification of at least one target site into the genome of the maize plant cell and
   b) introducing a polynucleotide modification template comprising the heterologous P450 nucleotide sequence into the plant cell.
28. The method of clause 27, wherein the site-specific modification is induced by a CRISPR-associated endonuclease.
29. The method of any one of clauses 26-28, further comprising growing a plant from the plant cell comprising the heterologous P450 gene sequence, wherein the plant exhibits increased resistance to southern leaf blight.
30. The method of any one of clauses 26-29, wherein the heterologous P450 gene sequence replaces the endogenous P450 locus.
31. The method of any one of clauses 26-29, wherein the heterologous P450 gene sequence is inserted into a locus different from the endogenous P450 locus.
32. The method of any one of clauses 26-31, wherein the P450 gene sequence encodes a protein comprising an amino acid sequence at least 95% identical to one or more of SEQ ID NO: 27, 30, or 33.
33. The method of any one of clauses 26-32, wherein the P450 gene sequence encodes a protein comprising the amino acid sequence of SEQ ID NO: 27, 30, or 33.
34. The method of any one of clauses 26-33, wherein the P450 gene sequence comprises a nucleotide sequence comprising at least 95% identity to one or more of SEQ ID NO: 25, SEQ ID NO: 26, SEQ ID NO: 28, SEQ ID NO: 29, SEQ ID NO: 31, or SEQ ID NO: 32.
35. The method of any one of clauses 26-34, wherein the P450 gene sequence comprises the nucleotide sequence of SEQ ID NO: 25, SEQ ID NO: 26, SEQ ID NO: 28, SEQ ID NO: 29, SEQ ID NO: 31, or SEQ ID NO: 32.
36. A method for producing a recombinant maize plant cell, the method comprising:
   introducing a P450 nucleotide sequence into one or more plant cells;
   inserting the P450 nucleotide sequence into the genome of at least one of the one or more plant cells; and
   selecting a plant cell that has inserted the P450 nucleotide sequence into the genome of the plant cell.
37. The method of clause 36, wherein the P450 nucleotide sequence encodes a protein comprising an amino acid sequence at least 95% identical to one or more of SEQ ID NOs: 27, 30, or 33.
38. The method of clause 36 or 37, wherein the P450 nucleotide sequence encodes a protein comprising the amino acid sequence of SEQ ID NO: 27, 30, or 33.
39. The method of any one of clauses 36-38, wherein the P450 nucleotide sequence comprises at least 95% identity to one or more of SEQ ID NO: 25, SEQ ID NO: 26, SEQ ID NO: 28, SEQ ID NO: 29, SEQ ID NO: 31, or SEQ ID NO: 32.
40. The method of any one of clauses 36-39, wherein the P450 nucleotide sequence comprises the nucleotide sequence of SEQ ID NO: 25, SEQ ID NO: 26, SEQ ID NO: 28, SEQ ID NO: 29, SEQ ID NO: 31, or SEQ ID NO: 32.
41. The method of any one of clauses 36-40, further comprising growing a plant from the plant cell that has inserted the P450 nucleotide sequence into the genome of the plant cell, wherein the plant exhibits increased resistance to southern leaf blight.
42. The method of any one of clauses 36-41, wherein introducing the P450 nucleotide sequence comprises bacteria-mediated transformation.
43. The method of any one of clauses 36-41, wherein introducing the P450 nucleotide sequence is accomplished via biolistic transformation.
44. A recombinant plant comprising a heterologous gene encoding a protein comprising at least 95% amino acid sequence identity to one or more of SEQ ID NOs: 27, 30, or 33.
45. The recombinant plant of clause 44, wherein the protein comprises 100% amino acid sequence identity to SEQ ID NO: 27, 30, or 33.
46. The recombinant plant of clause 44 or 45, wherein the gene comprises a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.
47. The recombinant plant of any one of clauses 44-46, wherein the gene comprises the nucleotide sequence of SEQ ID NO: 25, SEQ ID NO: 26, SEQ ID NO: 28, SEQ ID NO: 29, SEQ ID NO: 31, or SEQ ID NO: 32.
48. A recombinant plant seed comprising a heterologous gene encoding a protein comprising at least 95% amino acid sequence identity to one or more of SEQ ID NOs: 27, 30, or 33.
49. The recombinant seed of clause 48, wherein the protein comprises 100% amino acid sequence identity to SEQ ID NO: 27, 30, or 33.
50. The recombinant seed of clause 48 or 49, wherein the gene comprises a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.
51. The recombinant seed of any one of clauses 48-50, wherein the gene comprises the nucleotide sequence of SEQ ID NO: 25, SEQ ID NO: 26, SEQ ID NO: 28, SEQ ID NO: 29, SEQ ID NO: 31, or SEQ ID NO: 32.

### EXAMPLES

The following examples are offered to illustrate, but not to limit, the various aspects of the present disclosure. It is understood that the examples and examples described herein are for illustrative purposes only, and persons skilled in the art will recognize various reagents or parameters that can be altered without departing from the spirit of the disclosure or the scope of the appended claims.

### Example 1

In order to locate genomic loci responsible for SLB resistance, three SLB resistant inbred lines (CML304, CIMBL83 and K22) were used to generate recombinant inbred line (RIL) populations, with GEMS41 as the SLB susceptible parent. The RIL populations were grown, inoculated with Bipolaris maydis, and SLB scores were collected in Henan, China, and genotyped with genome-wide SNP markers. QTL analysis identified a major SLB resistant QTL within the same chromosome 3 interval (between SNP markers D6 and G3) in all three RIL populations. The QTL was named qSLB3.1.

### Example 2

CML304 and GEMS41 were used to develop a large BC5F2 population to fine map qSLB3.1. Approximately three thousand BC5F2 individuals were genotyped with markers D6 and G3 to identify recombinants within the QTL interval. The recombinants were scored as SLB resistant or susceptible and genotyped with additional SNP markers within the interval. qSLB3.1 was delimited to a 51 kb interval (B73 v4 reference sequence), flanked by SNP markers K6 and G8.

Initial markers determined for qSLB3.1 in the CML304 and GEMS41 genomes include those described in Table 3. Sequences in Table 3 are presented in the same manner as Table 1.

TABLE 3

| | SNP | Genome | | |
|---|---|---|---|---|
| | Linked | Position | | |
| Marker | with | (B73 | SEQ | Sequence |
| Identi- | Resist- | Version | ID | with |
| fier | ance | 4.0) | NO | SNP |
| D6 | C | 30,542,688 | 1 | accagaccgg |
| | | | | tccgatgcgc |
| | | | | cagctgtccg |
| | | | | agagccaatt |
| | | | | gcagaactct |
| | | | | [c/t]tgca |
| | | | | cacacg |
| | | | | gtccggtgtg |
| | | | | ccagctgata |
| | | | | gcccacttct |
| | | | | tgaatcttct |
| G3 | C | 36,339,580 | 12 | aattgaacca |
| | | | | gagaaggtat |
| | | | | ctaagaggag |
| | | | | ggtcttaccg |
| | | | | gagtatcgga |
| | | | | [c/t]agcg |
| | | | | cagggg |
| | | | | cccggtcgtc |
| | | | | gacggagggc |
| | | | | gtcacactcc |
| | | | | gtatgtttgg |
| K6 | A | 33,094,714 | 8 | aatgatatct |
| | | | | ctagaaccaa |
| | | | | atagctctaa |
| | | | | tggaatgata |
| | | | | tctctagaag |
| | | | | [a/c]aaat |
| | | | | acttgt |
| | | | | aaaagtctct |
| | | | | ttatatcctt |
| | | | | aagaatacca |
| | | | | tctagtaact |
| G8 | A | 33,145,490 | 34 | ataaagagca |
| | | | | caaataatga |
| | | | | catcgattga |
| | | | | aaatacttaa |
| | | | | acaacatggt |
| | | | | [a/g]gtag |
| | | | | aaaaat |
| | | | | accgtacggt |
| | | | | gcagtttttt |
| | | | | agaaagtaac |
| | | | | agttggcact |

A similar process was conducted to generate a BC5F2 population for K22 and GEMS41. After identifying recombinants and genotyping the recombinants, additional SNP markers were found for the QTL in the K22 parent. Without wishing to be bound by theory, it is believed that both QTLs (one in the CML304 and CIMBL83 parents and one in the K22 parent) contain the same causal gene. The markers in Table 1 represented by SEQ ID NOs: 1-12 are derived from CML304 and GEMS41. The markers in Table 1 represented by SEQ ID NOs: 13-24 are derived from K22.

### Example 3

Genomes of the three SLB resistant lines (CML304, CIMBL83 and K22) and the susceptible parent (GEMS41) were sequenced, and RNA-seq data generated to facilitate gene annotation. Two annotated genes were identified within the qSLB3.1 interval, with the first gene encoding a cytochrome P450 protein and the second gene encoding an apoptosis-antagonizing transcription factor. Comparative analysis of the qSLB3.1 interval between the resistant and susceptible lines revealed that the transcription factor is missing in two of the three resistant lines. The P450 gene is identical or near identical within the three resistant alleles (97.2-100% identical), while it is truncated and likely non-functional in the susceptible allele. It is concluded that the P450 gene is the only plausible candidate gene for qSLB3.1.

### Example 4—Transgenic Validation of the qSLB3.1 Candidate P450 Gene

Transgenic plants were generated with a construct containing the CDS of the P450 gene (consistent with SEQ ID NO: 27 and 30), with expression driven by a constitutive maize promoter. Greenhouse SLB assays were performed on segregating T1 plants from three different events. The results demonstrated that the transgene-positive plants have significantly enhanced levels of resistance to SLB, when compared to the transgene-negative (null) plants. These results confirm that the P450 gene is the causal resistance gene underlying qSLB3.1.

SLB scores, in all examples, are based on scale of 1-9 where 1 is most sensitive to SLB and 9 is most resistant to SLB.

TABLE 4
SLB scores of transgenic and null plants

| | Transgene positive | null |
|---|---|---|
| Event 1 | 3.7 | 1.0 |
| Event 2 | 3.7 | 2.3 |
| Event 3 | 3.0 | 1.0 |
| Average | 3.5 | 1.4 |

### Example 5—Transgenic Validation of the qSLB3.1 Candidate P450 Gene

Transgenic plants are generated with a construct containing the CDS of the P450 gene (consistent with SEQ ID NO: 27, 30, and/or 33), with expression driven by the native maize promoter. Greenhouse SLB assays are performed on segregating T1 plants from various different events. The results are expected to demonstrated that the transgene-positive plants have significantly enhanced levels of resistance to SLB, when compared to the transgene-negative (null) plants.

What is claimed is:

1. A method of identifying a maize plant comprising a marker allele associated with resistance to southern leaf blight, the method comprising:
screening nucleic acid from a maize plant for at least one of the following marker alleles on chromosome 3:
i. a “C” at D6, corresponding to position 51 of SEQ ID NO: 1;
ii. a “C” at C10, corresponding to position 51 of SEQ ID NO: 2;
iii. a “C” at A10, corresponding to position 51 of SEQ ID NO: 3;
iv. a “G” at G6, corresponding to position 51 of SEQ ID NO: 4;
v. a “G” at H6, corresponding to position 51 of SEQ ID NO: 5;
vi. a “T” at D10, corresponding to position 51 of SEQ ID NO: 6;
vii. a “C” at F10, corresponding to position 51 of SEQ ID NO: 7;
viii. an “A” at K6, corresponding to position 51 of SEQ ID NO: 8;
ix. a “C” at L6, corresponding to position 51 of SEQ ID NO: 9;
x. a “T” at 06, corresponding to position 51 of SEQ ID NO: 10;
xi. a “G” at Q6, corresponding to position 51 of SEQ ID NO: 11;
xii. a “C” at G3, corresponding to position 51 of SEQ ID NO: 12;
xiii. a “T” at D7, corresponding to position 51 of SEQ ID NO: 13;
xiv. a “T” at L4, corresponding to position 51 of SEQ ID NO: 14;
XV. a “T” at E7, corresponding to position 51 of SEQ ID NO: 15;
xvi. an “A” at G7, corresponding to position 51 of SEQ ID NO: 16;
xvii. a “G” at H7, corresponding to position 51 of SEQ ID NO: 17;
xviii. a “T” at I7, corresponding to position 51 of SEQ ID NO: 18;
xix. a “T” at J7, corresponding to position 51 of SEQ ID NO: 19;
xx. a “G” at K7, corresponding to position 51 of SEQ ID NO: 20;
xxi. an “A” at L7, corresponding to position 51 of SEQ ID NO: 21;
xxii. a “T” at M7, corresponding to position 51 of SEQ ID NO: 22;
xxiii. an “A” at N7, corresponding to position 51 of SEQ ID NO: 23; or
xxiv. an “A” at O4, corresponding to position 51 of SEQ ID NO: 24,
wherein the presence of the marker allele is associated with resistance to southern leaf blight.

2. A method of producing a maize plant comprising a QTL associated with resistance to southern leaf blight, the method comprising:
a. screening a maize plant population for a QTL associated with resistance to southern leaf blight, wherein the QTL comprises one or more marker alleles selected from the group consisting of:
i. a “C” at D6, corresponding to position 51 of SEQ ID NO: 1;
ii. a “C” at C10, corresponding to position 51 of SEQ ID NO: 2;
iii. a “C” at A10, corresponding to position 51 of SEQ ID NO: 3;
iv. a “G” at G6, corresponding to position 51 of SEQ ID NO: 4;
v. a “G” at H6, corresponding to position 51 of SEQ ID NO: 5;
vi. a “T” at D10, corresponding to position 51 of SEQ ID NO: 6;
vii. a “C” at F10, corresponding to position 51 of SEQ ID NO: 7;
viii. an “A” at K6, corresponding to position 51 of SEQ ID NO: 8;
ix. a “C” at L6, corresponding to position 51 of SEQ ID NO: 9;
x. a “T” at O6, corresponding to position 51 of SEQ ID NO: 10;
xi. a “G” at Q6, corresponding to position 51 of SEQ ID NO: 11; and
xii. a “C” at G3, corresponding to position 51 of SEQ ID NO: 12;
or one or more marker alleles selected from the group consisting of:
xiii. a “T” at D7, corresponding to position 51 of SEQ ID NO: 13;
xiv. a “T” at L4, corresponding to position 51 of SEQ ID NO: 14;
XV. a “T” at E7, corresponding to position 51 of SEQ ID NO: 15;
xvi. an “A” at G7, corresponding to position 51 of SEQ ID NO: 16;
xvii. a “G” at H7, corresponding to position 51 of SEQ ID NO: 17;
xviii. a “T” at I7, corresponding to position 51 of SEQ ID NO: 18;
xix. a “T” at J7, corresponding to position 51 of SEQ ID NO: 19;
xx. a “G” at K7, corresponding to position 51 of SEQ ID NO: 20;
xxi. an “A” at L7, corresponding to position 51 of SEQ ID NO: 21;
xxii. a “T” at M7, corresponding to position 51 of SEQ ID NO: 22;
xxiii. an “A” at N7, corresponding to position 51 of SEQ ID NO: 23; and
xxiv. an “A” at O4, corresponding to position 51 of SEQ ID NO: 24;
b. selecting from the population at least one maize plant comprising the QTL;
c. crossing the maize plant selected in (b) with a second maize plant; and
d. obtaining a progeny plant that comprises the QTL.

3. A method of selecting a maize plant comprising resistance to southern leaf blight comprising:
a. screening a population of maize plants for the presence of (1) a gene encoding a protein comprising at least 95% amino acid sequence identity to one or more of SEQ ID NOs: 27, 30, or 33 or (2) a protein encoded by a gene comprising a nucleotide sequence with at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32;
b. selecting a maize plant comprising the gene;
c. crossing the maize plant selected in (b) with a second maize plant; and
d. obtaining a progeny plant that comprises the gene.

4. A method for producing a recombinant maize plant cell, the method comprising:
introducing a heterologous P450 gene sequence into the genome of a maize plant cell and
selecting a plant cell comprising the heterologous P450 gene sequence encoded in the plant cell's genome,
wherein (1) the P450 gene sequence encodes a protein comprising an amino acid sequence at least 95% identical to one or more of SEQ ID NO: 27, 30, or 33 or
(2) the P450 gene sequence comprises at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.

5. The method of claim 4, further comprising:
a) introducing a site-specific modification of at least one target site into the genome of the maize plant cell and
b) introducing a polynucleotide modification template comprising the heterologous P450 nucleotide sequence into the plant cell.

6. The method of claim 5, wherein the site-specific modification is induced by a CRISPR-associated endonuclease.

7. The method of claim 4, further comprising growing a plant from the plant cell comprising the heterologous P450 gene sequence, wherein the plant exhibits increased resistance to southern leaf blight.

8. The method of claim 4, wherein the heterologous P450 gene sequence replaces the endogenous P450 locus.

9. The method of claim 4, wherein the heterologous P450 gene sequence is inserted into a locus different from the endogenous P450 locus.

10. A method for producing a recombinant maize plant cell, the method comprising:
introducing a P450 nucleotide sequence into one or more plant cells;
inserting the P450 nucleotide sequence into the genome of at least one of the one or more plant cells; and
selecting a plant cell that has inserted the P450 nucleotide sequence into the genome of the plant cell,
wherein (1) the P450 nucleotide sequence encodes a protein comprising an amino acid sequence at least 95% identical to one or more of SEQ ID NOs: 27, 30, or 33 or
(2) the P450 nucleotide sequence comprises at least 95% identity to one or more of SEQ ID NO: 25, SEQ ID NO: 26, SEQ ID NO: 28, SEQ ID NO: 29, SEQ ID NO: 31, or SEQ ID NO: 32.

11. The method of claim 10, wherein the P450 nucleotide sequence encodes a protein comprising the amino acid sequence of SEQ ID NO: 27, 30, or 33.

12. The method of claim 10, wherein the P450 nucleotide sequence comprises the nucleotide sequence of SEQ ID NO: 25, SEQ ID NO: 26, SEQ ID NO: 28, SEQ ID NO: 29, SEQ ID NO: 31, or SEQ ID NO: 32.

13. The method of claim 10, further comprising growing a plant from the plant cell that has inserted the P450 nucleotide sequence into the genome of the plant cell, wherein the plant exhibits increased resistance to southern leaf blight.

14. The method of claim 10, wherein introducing the P450 nucleotide sequence comprises bacteria-mediated transformation.

15. The method of claim 10, wherein introducing the P450 nucleotide sequence is accomplished via biolistic transformation.

16. A recombinant plant comprising:
(1) a heterologous gene encoding a protein comprising at least 95% amino acid sequence identity to one or more of SEQ ID NOs: 27, 30, or 33 or
(2) a heterologous gene comprising a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.

17. The recombinant plant of claim 40, wherein the protein comprises 100% amino acid sequence identity to SEQ ID NO: 27, 30, or 33.

18. The recombinant plant of any one of claims 40-42, wherein the gene comprises the nucleotide sequence of SEQ ID NO: 25, SEQ ID NO: 26, SEQ ID NO: 28, SEQ ID NO: 29, SEQ ID NO: 31, or SEQ ID NO: 32.

19. A recombinant plant seed comprising:
(1) a heterologous gene encoding a protein comprising at least 95% amino acid sequence identity to one or more of SEQ ID NOs: 27, 30, or 33 or
(2) a heterologous gene comprises a nucleotide sequence comprising at least 95% nucleotide sequence identity to one or more of SEQ ID NO: 25, SEQ ID NO: 26, SEQ ID NO: 28, SEQ ID NO: 29, SEQ ID NO: 31, or SEQ ID NO: 32.

20. The recombinant seed of claim 44, wherein the protein comprises 100% amino acid sequence identity to SEQ ID NO: 27, 30, or 33.

21. The recombinant seed of any one of claims 44-46, wherein the gene comprises the nucleotide sequence of SEQ ID NO: 25, SEQ ID NO: 26, SEQ ID NO: 28, SEQ ID NO: 29, SEQ ID NO: 31, or SEQ ID NO: 32.


SEQUENCE LISTING (34 sequences)

SEQ ID NO: 1 - DNA, 101 bp, Zea mays
  source 1..101: mol_type = genomic DNA
accagaccggtccgatgcgccagctgtccgagagccaattgcagaactctytgcacacacggtccggtgtgccagctgatagcccacttcttgaatcttct

SEQ ID NO: 2 - DNA, 101 bp, Zea mays
  source 1..101: mol_type = genomic DNA
cacgtcgaccctgaccctgtacttgtaccgcttggcaaagaagacgaaaaygagcaggttgacggtgctgagcacggcgagcagccagtacaggtagtcga

SEQ ID NO: 3 - DNA, 101 bp, Zea mays
  source 1..101: mol_type = genomic DNA
acaccaaatttagtagcactagtgcgatgggacagtccggcttgttagccyggaccatccagaagtaaattagttcgagcgagtcttgatttgctacgaga

SEQ ID NO: 4 - DNA, 101 bp, Zea mays
  source 1..101: mol_type = genomic DNA
accgatggttttctctacgcgcgcgctcccttcccctctacctctgactastgggaccggtctgtcggcaccagccgccctcgctcgcgtgcccgctccct

SEQ ID NO: 5 - DNA, 101 bp, Zea mays
  source 1..101: mol_type = genomic DNA
aaacagccacattttgcatacatatcaagaagggcactgcctgagaacacrctggacatgtgccctgatgttgtgaggtaagcatgcaattgccttcctat

SEQ ID NO: 6 - DNA, 101 bp, Zea mays
  source 1..101: mol_type = genomic DNA
aaaagaaattaaaaggaaaaaactaatccccacggcaacaagacaaccaaycagttcaaccctgcaactgcaggggtattattgtcgcaggctcgcctata

SEQ ID NO: 7 - DNA, 101 bp, Zea mays
  source 1..101: mol_type = genomic DNA
tgtctcagctatcgaaggagttaaagctattttgatgtgaggtcttatttmtgaacttgtagtttgtcattaaagtttatatattgtgatagtcgtagtgc

SEQ ID NO: 8 - DNA, 101 bp, Zea mays
  source 1..101: mol_type = genomic DNA
aatgatatctctagaaccaaatagctctaatggaatgatatctctagaagmaaatacttgtaaaagtctctttatatccttaagaataccatctagtaact

SEQ ID NO: 9 - DNA, 101 bp, Zea mays
  source 1..101: mol_type = genomic DNA
agaagtccagacatatatcatcactatagaggtgctcgtcaagaggcatgytgctcatgttaaacagttggtcaaaaaagttcttattgtcgtccaaagta

SEQ ID NO: 10 - DNA, 101 bp, Zea mays
  source 1..101: mol_type = genomic DNA
cttcagtgccccctccttggtccctagcgcggcggcgcctgcaacagggayctctatggctccacgattactatcagcttccggctgtgttgaaaaccatt

SEQ ID NO: 11 - DNA, 101 bp, Zea mays
  source 1..101: mol_type = genomic DNA
aagtgattattaaagagtagcattttggattgagtcatgtggactaatcakagtgttagtattatgtaacacatctctttgcttgtagtgcacatgtgtgg

SEQ ID NO: 12 - DNA, 101 bp, Zea mays
  source 1..101: mol_type = genomic DNA
aattgaaccagagaaggtatctaagaggagggtcttaccggagtatcggayagcgcaggggcccggtcgtcgacggagggcgtcacactccgtatgtttgg

SEQ ID NO: 13 - DNA, 101 bp, Zea mays
  source 1..101: mol_type = genomic DNA
tatgaatggagtattacacttaggtatttagcacatcatcatccaccgtaycttgttcccctgccaagttcctactgaggtagactcataatcagtctttc

SEQ ID NO: 14 - DNA, 101 bp, Zea mays
  source 1..101: mol_type = genomic DNA
ctcccggagctcaagaactgaatgaaactattgctgctgtagctttagctwgctaaatgcctgcatgctgcaccagctgttgtatctcccagctctcgaag

SEQ ID NO: 15 - DNA, 101 bp, Zea mays; identical to SEQ ID NO: 2
  source 1..101: mol_type = genomic DNA

SEQ ID NO: 16 - DNA, 101 bp, Zea mays
  source 1..101: mol_type = genomic DNA
gaaaattcagtctcaaatacaggcagcacacctagtttccctgggaacacraaatcgaaaagagaccagagctcagaaagtttattttgaataggtgcacc

SEQ ID NO: 17 - DNA, 101 bp, Zea mays
  source 1..101: mol_type = genomic DNA
cagtggaacatatactgcttgctcacaaaatgagttcttgcttgcttacakgctaatgatggtgacaaaaatcagatctaaagaggagtaccttacaatca

SEQ ID NO: 18 - DNA, 101 bp, Zea mays
  source 1..101: mol_type = genomic DNA
ccatccgcgagatgaggaatagacgagacaagctcggagggatggctgtcygacgtccaaaggcagggcgtctttgcgtctttccacgcggaagctaggct

SEQ ID NO: 19 - DNA, 101 bp, Zea mays
  source 1..101: mol_type = genomic DNA
ccttcttccccgtggaaggtgcagcaactgctgccgctgtgcgcggtcctygccgtcgtcgtcgcgtggtgtgccgcgcgcgcggcggagtgggcgtggct

SEQ ID NO: 20 - DNA, 101 bp, Zea mays
  source 1..101: mol_type = genomic DNA
cagctgacgcggctgctgcactatggcgtgagcacccacgagggcagcaartgggccaagcaccgcaggatcatcaaccccgctttccatttcgagaagct

SEQ ID NO: 21 - DNA, 101 bp, Zea mays
  source 1..101: mol_type = genomic DNA
agcggcggcggaagtgcgccggcgggctcgtgggcgggagcgagaggatgrggtgggacccaagctgcgtcggggaagggtagaggatcctggccctgggg

SEQ ID NO: 22 - DNA, 101 bp, Zea mays
  source 1..101: mol_type = genomic DNA
ggataggatgtgatattacacgctgtttctcgatgtagcgggatgctcatyaccctagttttgaggggtattatggtttaggggcgctcaagttggtcaga

SEQ ID NO: 23 - DNA, 101 bp, Zea mays
  source 1..101: mol_type = genomic DNA
gacattaatgttttcgctcagatgattgacgacgcggaaggcctggaaaaratcgagaacctacagagccatgataataatgaaatctatgaaaaggctgt

SEQ ID NO: 24 - DNA, 101 bp, Zea mays
  source 1..101: mol_type = genomic DNA
acgtattttacttggtacctccttagcttgatgtcaccttactatatctgwaatcagaagcatagcctgtggtgcatggccggagctaagatactacaagg

SEQ ID NO: 25 - DNA, 12786 bp, Zea mays
  source 1..12786: mol_type = genomic DNA
gcgtttcgagactagggggtccctgggccgacgagtgaatgtcgccgcgtgccccagcccagatgggtcggcgcgaggccgagcgcgaaagggggaagtgaggtggccggagacgggcgtgagagaggtggaaatcccgcggccttcgtgttcgtcccgcgcccaggtcgggtgcgcttgcagtagggggttacaagcgtccacgcgggagagggagcgagcggcctcacgcgagcgcctgtctcgtcctcgtccccgcgcggccaaccctgtgtaagagggccctggtccttccttttataggcgtaaggagaggatccaggtgtacaatgggggtgtagcagagtgctacgtgtctagcggaggagagctagcgccctaagtacatgccgtcgtggcagccggagagattttggcacccggttcgtgtggtgtcgtggccgtcggaggagcgctggagcctggtggaaggacagctgtcggggctgtcgagtccttgctgacgtcatcttgcttccgtaagggggcggagagctgccgtcatcagggagcgtgcggggcgccatcattgcctatctggcggagcgagccagatgggacgccggtcttgttccccgtagcctgagtcagcttggggtagggtaatgatgacgcctcctgttgacgtggtcggctcgcgcccgaggttgggcgatgtggaggctcctccgaggtcgaggtcgagtctgtcttccgtggccgtggtcgagtccgagcctctgggtcgggcgaggcggagaccgtcggctgaggtcagggctgagtccgagccctggggtcgggcgaagcggagttcgtcgtcttctggggctgagcccaagtccgagccctgggtcgggcggagcggagttcgtcgtcttctggggcttagcccgagtccgagccctgggtcgggcggagcggagttcgccgtcttctagggcttagcccgagtccgagccctgggtcgggcggagcggaattcgccatcttccggggcttagcccgagtccgagccctgggtcgggcggagcggagttcgccgtcttcaggggctgagcccgagtccgagccctgggtcgggcgaagcggagcttcctatggtgcctgcggccgggcctgactgcctgtcagcctcactctgtcaagtggcaccgcagtcggagcggcgcaggcggcgctgtctttctgtcaggccggtcagtggagcggcgaagtgacggcggtcacttcggctctgtcgactgaagggcgcgcgtcaggataaaggtgtcaggccacctttgcattaaatgctcctgcgatttggttggtcggcgcggcgatttggtcagggttgcttcttggcgaagacagggcctcgggcgagccggaagtatgttcgccgctggaggggggcctcgggcgaaacggagatcctccgggatcggctgcccttgtccgaggctaggctcgggcgaggcgtgatcgagtccctcgaatggaccgatcccttacttaatcgcacccatcaggcctttgcagctttatgctgatggggttaccagttgagaattaggaaccttgagggtactcctaattatggtccccgacaccaggcgatggcttctgcaagaaatgtcgtggcttacggtgcaagctagtatatttacaaagggatatttcgacatgcaaaaaaactcgtgccgtttaaaggacaactagaaatgcgacatttgaagtcagaatgcataatataattaaaaaatatatattttcgaaatgaatattatgttacacatgtttgactaaaaaataaattctaaataaattaaaaaaccattttgtgcatgatttatttgaattagcgtgtaatgaaaaaacaaataaatattgttgctttagaagactgaatttaggttgttggagaccgagaaaatatatatgacataattttttataatgtgttgtaaaggatgaaaaatatttatttagggatagaatttaggggctggagagtggagacggtctaagcgtataggacgacggcgatggcgagcagctctctccgttcggttccccttccgctgcacacagaaaagtcacgagcttagatttttttcacccaagtggctggcggctagccaagcgcccaagtcatggcgacgagcgtgccagagggattgctcggcgagccttcttccccgtggaaggtgcagcaactgctgccgctgtgcgcggtcctcgccgtcgtcgtcgcgtggtgtgccgcgcgcgcggcggagtgggcgtggctgaagccgcggcgcctggaacgggcgctgcgggcgcagggtatccgtggcacggcgtaccgcccgctggccggcgacgcgccgctgtccgatcgactcgcccgcgaggccaggtccaggccgcccctaccgccgggctgccacgccatcgtgcccagagccatgccgctcgtccaccactccatgaacgagcacggtaacttctctctcgctgttgtcgcgttgactacttgactctaaccggtgatctacctttagcttgccggtcgtaccacaccaactcactaaggaacgaacggtttttccatggtattacacaaacatttatcatagtaaatttcaaaatataaatcttatgtcatgaagttacataagttatttaataaaaatataaatatttgatttagaataaagctaatacaaagtgtgctgtgggaaatatggtttcaagtagtttgcttgacatcgaggatcattgagttattggtactgattaagatatcaattagcgaataaatttcccctccagtgtggaatattaaaaaaaaaatagagtgcggaggattcaaccctgctcgcttaaggccctttggcacggctccagctcgagcttatagtgaagcctagcaaatggtcaaaaaaatggcttctggtcgatagtcctctaagagctggagtcgttcttttgttgggagcaaaagcataaaaaatatggcttctagcagctcctatatttttctcatacatcattcgaaaaacatatgagaaattattttgccaaacgaattgtaaaatgactttggctccttcagagaagccaaagagccagaggagtcagagccaaaactattttagaggaggcggagcgctggcaaagggccatagtataaagcacttggcttgtacaattgcaagtcgtgtttgtttatgtacatgttacgtaaagaagagatattgtaaatatatacatatagtaataatttgaaaataaaaaatataatcatgtttgaataaatatctcaattaagagaggactactccattatatatagaaaccgtagcaataactagttgccatatactttgctagaagttgcatgttattcactaccggatagatctgctttgccgagtggcaaagcccaggaaactctcggtaaaatctttgccgagtgtgactctctgtaaagaagtctcggcgaactgtacatcggcaatggcttctttgctgagtacgttttgtcgggcacttggcaaagaaaagttaatgtcacggcgacaagggacggtgatgcagcctttgtcgagtgtcctctgtgacactcggcaaagaagcgttctttaccgagtgcttgttggcctgacattcggcaaagaaagctctagtgggcctctttgtcagtccctttgtcgagagctctggtaggcactcggtaaagaagctccctttgtcgagtgcctgttggactgccactcagcaaagaaagctccagtgggcctctttgccggttcctttgccgagtgtgctgggaggcactcggcaaagggtgcttatttaccgagtgctgcgaccatatcactcgacaaagaagctttatcggttcccaagtgtcttctttgtcgagtgctttggttatcgcactcggcaaagaacctctttgccgagtgtaacactccggaaattgaccagaaaaaaccttttttatttgtttttattattccatccaaacaaacaaaggatatatatcattgacatcacatatatcacataatcaacacatgtatcaccaacaccataatctcacaaatatcaccacatcaaacaagtttcacaaacataaatctcaccaagtatctcacaacatcaagtattatcaacatcaataagtttaacaaacacaaatatcaccaacactcgcaaacataagtctcaatcatcactaagaggttatcaacaaggtgggcggatggactgttttagcgaagggttgggcaatctgtgagggttatttgatgtcgccgattatccctgcatagagaagagattgcatttgtgagaccagatgaatatatattatgtaggactaaaaatttgatactcacaggagtatggaactgagcaaggtcagctggagggaacaacggaggtggtggagcaagaccctgtgcagcgccaaagctctgcatgtactggaacatctctgccatcctctgctgattcgccaagcgaaccacccgctccatcctcatcctcgcctctatctccatgtgtccctccctcttcttctagctgggcctgtaatatctcaccccaatgttataataatgcaaagagaaggtatataactcaatgaatgacaaggcatagaagcactaacctggagttccaagatacaatgttgggaactatcttgccaaaaggtcgtatggctaggctcgcgctcatactccttgctcacacctgagacagagtgggagtggaggatgagttgattgccccgtcagcaatccagtaccgcccatgcctcttgcctcctccgaccctcatgaggacatctctatcgatgtccttagtcctcggattgtaatctagcccatggacctcctttgccatggcagtgtactcactgaggcggctacggacggtaggttgctataagcctcaggcttgtgctccaggttgtaggtgacatcggacgtcgccttgcccttgtgggccatagcatacgtcgagaagattgagctaggctagccaccatgtgacgtcggctacgataaaaccaacgagatggttagaaatcatgcctaatcaaaagttagactaaattaaagaaagtgcatatacatgcttctatgtatttgccaaggctacgactgccttgatggtgggagggaccttgcatcatcaaactccgttcctggctagcgttgtgcgcctcgtcccactcggccgagcaccacctttgcaccatctgctccaagcactcaggatgcgcgatgcaccaataaggaataatcttcaaagaaaacaattacacaacatatcaaaagataaaattaagcatatttaatatcaatattaatgttctgtatttacctgcaagtactggttcagagtcaatgacatggttcgggcgtccttgctcaccttctccctaaggatggagtcgtggtaagtgatgatggcctagatgcacgcctcgtactgcatgtccacgatgagcttcttatggctcgcggtagccaccacatccatcgtggcctcatttctagcctcgtagctaaagatatcctacgtacaaagacgatgtattcatacattatttttaagaatgtgcaataaatgcaacatattaaacaatatagtgcaaggaagacttacccacagctcttgcttcatccgctccgccttgttgttgaattatctatcatctcgatccactgcatcgggggcgacagtgtagtggtcgaaggaataaaccaagcccatcactccggcatactcaacaagtctaggaaagtgttccctgcacagaagcccgaggatgccattggggttgcggcgtgacccctgcattcttcaaaacctttcaagatatgtctaagtgattaagaaaaattatcagtttcttttttaaatttgaacatataatatgaaaaagtatcgacaacatctaaagttgcatacctttccccatcgggtcgaatgagcgggcacctgtcacgaagtatcggtcgctgaaggagactcgcgagacctcgcaagtagacgtgcatcgaacatgtggtgccagacctagaggcgacccgctgagcgtcttcagtcctactgtgcctcctcctccatcctacgggtgctcctcatcctcctctctctcctcgtcccaccgcccaccatctttgtctaaccacctgcaattaagagtaagcaattaagcctagataaaaaatatgtatttagaaacatatgtgaaataaataaaatatagcattacattgattaaaataatcttcatatgtgttcggattagccagatcataagtctcatcatcattatcaaccatttcatagtcaacatcatatgaaggcacaagtttataattgtaattgccttcaagcatttctaagtcattctcattttgcacttcattttcctcctcatcaacaaccatttcaatatctaccatccatttcgattgctttggttaaatctatctcaaatcgctcttctagctattttcttaaaagaactctctgtcatatgtctctgggtctaagttataatcttcatcgtttagaataggtaatttatcgtgcagtgataccatatacacagcatctcaacccttaagatattcttttatttggcacacatatgggagataatatacttatgtggtctgttggttcacgatatagacatcgtctcctggtaaggtggaatcctatcgaatttcgattagcccaagattagaatgtgtccatctcgtcacttcagcgtcaaaccaatgacatttcagtatgaactgaaggtggcgtaggattgtcataaggattttgagctgcctcttttgtccatcacccgattctacctctatgaacctagacgatttacacttcagacggtactttgcctctgtgtattttttcctaaataggacacaacccttcagacaaacatgtgtctgctcatacatcatcttgagtgcacgaagaagtttctatgcctcgtacatgctctttggcagaatgtgatcctctagaagcaaactgccaataactatcaacaatccatcaaaggtgtctcgacttaggctgtaatgcgacttgaactccattatacatccaatggcatccagttgagaaacttttgtcttgccatgaaggggtttctgtgccacgtcaaacatgtcgtagaacgcctttgtggtcgcctctagctcgtcctcagtatgtgattcagcgaattatgcctcgtgatagtcgtttaccgctaccttggcaccaacatcataaccctcgatgcgttgtctcgccacctcctctctcgtacgatgcgcttcaccatggaagatccatcgagtatagtctggcgtaaatccattcttccaaatatgttgtctcatggctttctttgtttttcttttcttgttgtcacatttgttgcacggacatggaactagactcacgcctttagcagcttcgccaaatgctcgttccaagaaagcatcagtctttgtaatccattcaggagtgacatcattccttgttacgtggtccatgtacatccactcacgatccttaatactctaacatatataagcaagtatagtctaatataaaaatgtaatgcatatacactatgttcctaccttctaatgggtgaggataggtcctaatcctacccgcagttgcgtagatggggttagtttccatgctctactctgatccaagacagaatttcggcagcacctctccgttgttctatggaaacacatcctagctaggagactgtgtatccagagaacaacagggaggtgatgttgaaactctatctcgaatcggagtagagcatgtaaactaatttcgtctacgtaaccgcgggctatccaaaaaatatggacaattcgaaacatatacacatttgtagatatgtaaagatccacatatctatatcttatctctttcgaacgggagacgcctaactgggttacgtgatctacgaccatgacacagaaatcaaggttataccttaggtggcggtggagtcaggctagcggggctgtggtgcgtcggtgcagtggcaacgtgacacggtgcaggtagacccacaacagctaggaagccctctgcaaaaaaggaaaacaagtctgtcaagacaaaatttcgacagcacctttcctacatggtgatgttttcaaaacctgcaaataaaactaacagcacgatggctgatatccacacatatagatcaaggccttgataaccacatatacatatttgatctcacatattcatgcatgtttgttctcacaattcacaatcacacaaaaaaatagttcactaataataaatttcaaactcatagttcaaaataacttaacacaacttcacaagtcacaatttcacaataacaactaaaaataactaggaccaaacatcaattactagccaaacatcatacaatttctatgtgtgttgcaaatgacaaaaaagagtctttaactttgcttggccggagcacaggagagggcgcagatctaaggtggcggtgtgttagccggagcgggggaccaattgatggggatggtcggagccagcggggacgaggggccggagtcggtgtggacggtggcgccctggtcgatggggacggtggcgcgctggcgctcgagtgggtgacggggccgaagtcggcgtggacggtggcgctggccgacaaggacggtggtgcgctggcgctcgggcgggcgacgcgagagcggtgaggtgctggcgtggcggcgagcgggtggggtgcatggcaacggcaggcagcggttgagcgtgcgaaagaatgggaccacgggctttggtcattgttaagtaaacgttctttgccgagtgctcgcgacctcacactcggcaaagattttttttaaaatttaaaaatgttactttgccgagtgccatggatctgacactcggcatagtatactttaccaagtgttctggatctaacactcggcaaagattattttgatttgaaaactactttgttgtgtgttcctctgtagtatccaagacactcggcaaagtatatttttatttttatttttattcttttttcattaaactttttgtggtttgttcctatactatatagacctacatgttccattttagtataattatcaaagtgtttgctataaatattagatttagttcgtttaattgaattttttcggataattcagatttaaacgaaaaatggaaaacagtgaatgcaaaaatgatttcatgttatttagcacaagttacggtctatttcataaacaaaccagaattttcgagcaccatgcttaaacatgaccatcaacctgtgatccagttattttaaaattgtataaaacacaaacaaaatcagcgaatcatgaaacttgtcaacatgtcatgatatcatatatagagaatctgataaaattttaaaactatttcatgaaagttgttacgcactatacgtagaaaatctagctgaagttaatagaagttttatgattttacgtgaagtccgactaattttgactcgtatgactcatgacccatccactctggattgacaacgcgtggcctactggtctgggcactggccaggcaagacctcggtcacctggttcgggccgacgccgagggtgatcataaccgagccggagctggtgcgccaggttctgtccaacaagttcggccacttcgagaaggccggcttcgggcagctgacgcggctgctgcactatggcgtgagcacccacgagggcagcaaatgggccaagcaccgcaggatcatcaaccccgctttccatttcgagaagctcaaggtctcgtctcatgcgtgaatcacctcatcaatagcttgctagttgatggatctgatacgagcttcccgatcgaccgattctcctgcagcgcatgctgccggcctttgcgtcgtgctgcgccgacctggtgagcacatgggagcgtctggtggtcgcggccgacggcgaaccatgcgaggtggacatctggccggagatgcagaggctgacgggggacgtcatctcccgcgtcgccttcggcagcagctacctggaaggcaggaggatcttcgagctccaggaggagcaggtgcacctcgccatgctcgtcgccggcaagatacacatccctggatacatgtcagctcgtgctctctagctagctctgatcaaactacattctatattaatttgattgtcagtgagtaactgacacgacaaaaagattttgatatacagaattgcaattttcgtgcgtgtaggatgctgcctacaagggtaaaccggcggatgaagcggatcgcggcggagatcgaagggatccttagacggatgatcgtgacgagggagagctcgctgagggcaggaaaggcgacgagtgacgaccttctcggcctgctgctagagtccaacatggagcagctcaggggccaaggcgaaggcgaaggcggaggcggcgggacgagcacgagctccggcggcttgtccaccgacgacatcattggagagtgcaagctgttctacttcgccggcatggagaccacgtcggtgctgctcacgtggactatgctggcgctctgcatgcacccggagtggcaggaccttgccagggaggaggtgctccgcgtcttcggcggcgcctcctgcagagtgccggactacgacggcctcagccgactgaaaatcgtaaccttcatttcgatagcaggatcagtcttgatcgattcgtccagtcagtgtcgtgtgaaatgtggtcgtaattgtgattcgcaatcgcgtctgcaggtgaccatggtgctgtacgaggtgctgcggctctacacgccgctgccggcgttgcaccggcggacatacaagcccatggagcttgggggcgtcaggtacccggcgggcgtgatgctgatgctgccgctgctctgcatccaccacgacaaggacgcgtgggggccggacgccgacgagttcaggccggagaggttcgcggagggagtcgccagagcgtcgtccaccggggacgcgccgccgtcgttcttcccgttcggctggggacctcggacctgcgtcggccagaccttcgcgctgctggaggccaagatagggctcgccatgattctggggagcttcacgttcgagctctccccgtcctactcgcacgcgccgttccccgtcgtcttgctcaagccagagcacggcgcgcaggtcaagctcaggaagctccaatgcatgagcagcatccgcatgcacgctgcacagccgaaacggtcataaatttccctgtatatatatgcgaaaaaaggcaacaacatgtatggatggaccaggactattgttagttaatgcctaaaaatactctatgcgtcctaaaataagtatccgtatgtttaaggtgaatagatcaatgtctttaagaaattatatgtccacctataaatatgtaactattgtacttcggaaaaatagaaggtagaaaagaacaactagagttagtttatcttgtaaatcgaggtgcacttattttgtcacagattttaaattctaggagtacacttattttgggatgtacaaagtagtcggctaaaacttgtcaactagttggtagtacataattgtttggattgtcaactaattattagaccttttgtgaacataaggttgatatgtgctcttgtgttttggttgtgatgtatgattatcaacaagagcagtgtctttgggttaaatcgtgtggactaatcggatgtgagagtatttgtgcaatatatctcttgtcttgtggtgtgtaggtttgaagtcttgagatggtagtcgacggcaagggtgaaagtcatgcggaattgcgtcgatggactgggagtagtaaagaacgtacactgggtcttgactgagggactagagaagctagaagactagttatggcggttgacacctgtggacactgataggtgtaaggatatggaggagtggatcgtatcattgacgtggttgtggaccgacggaacacatgtccaagacgtgggatcacgtatgtagtacgctaactatgggggtttaggtgattaagcctcaaaaccacccaacacaagttcgcgggtttcatcggatttttctcaaaactagatggcatggttccgaagggaataggaagcgacatgtggcatcatcatgaatggtgtgccaaggcaaagcaatttcgtgaagagtgtatgataatcatatcaaaattccaagagctagtccattttgcccctatatctagtggtagtttaggatagaggaataaccctttataaataggtaggggctgaaccattcaaccatctctctagcacctctgtaatttgtattctttatttccttctcattttgttaggagtccttagatgctttgaactttaatatccaagttcatgcaattttgaggaaatgtggccataaccacactttgccttttgggttttcaattcgtgtcaaatttaataggttttttctagttattgaggttttgagttgatcttggtcggtactgacccaaacaaccgatcgaccgaccacatgctacgccatattatcaaggatgtgtgacatggtttgaagccgatttagggtctatttggttcaattgtagattcttcaaaatctaattctagccgtgagctataaaaatattaaaatgagctaacgattataagaaaaataaaatatgtttggttgaaataactgc

SEQ ID NO: 26 - DNA, 1710 bp, Zea mays
  source 1..1710: mol_type = other DNA
atggcgacgagcgtgccagagggattgctcggcgagccttcttccccgtggaaggtgcagcaactgctgccgctgtgcgcggtcctcgccgtcgtcgtcgcgtggtgtgccgcgcgcgcggcggagtgggcgtggctgaagccgcggcgcctggaacgggcgctgcgggcgcagggtatccgtggcacggcgtaccgcccgctggccggcgacgcgccgctgtccgatcgactcgcccgcgaggccaggtccaggccgcccctaccgccgggctgccacgccatcgtgcccagagccatgccgctcgtccaccactccatgaacgagcacggcaagacctcggtcacctggttcgggccgacgccgagggtgatcataaccgagccggagctggtgcgccaggttctgtccaacaagttcggccacttcgagaaggccggcttcgggcagctgacgcggctgctgcactatggcgtgagcacccacgagggcagcaaatgggccaagcaccgcaggatcatcaaccccgctttccatttcgagaagctcaagcgcatgctgccggcctttgcgtcgtgctgcgccgacctggtgagcacatgggagcgtctggtggtcgcggccgacggcgaaccatgcgaggtggacatctggccggagatgcagaggctgacgggggacgtcatctcccgcgtcgccttcggcagcagctacctggaaggcaggaggatcttcgagctccaggaggagcaggtgcacctcgccatgctcgtcgccggcaagatacacatccctggatacatgatgctgcctacaagggtaaaccggcggatgaagcggatcgcggcggagatcgaagggatccttagacggatgatcgtgacgagggagagctcgctgagggcaggaaaggcgacgagtgacgaccttctcggcctgctgctagagtccaacatggagcagctcaggggccaaggcgaaggcgaaggcggaggcggcgggacgagcacgagctccggcggcttgtccaccgacgacatcattggagagtgcaagctgttctacttcgccggcatggagaccacgtcggtgctgctcacgtggactatgctggcgctctgcatgcacccggagtggcaggaccttgccagggaggaggtgctccgcgtcttcggcggcgcctcctgcagagtgccggactacgacggcctcagccgactgaaaatcgtgaccatggtgctgtacgaggtgctgcggctctacacgccgctgccggcgttgcaccggcggacatacaagcccatggagcttgggggcgtcaggtacccggcgggcgtgatgctgatgctgccgctgctctgcatccaccacgacaaggacgcgtgggggccggacgccgacgagttcaggccggagaggttcgcggagggagtcgccagagcgtcgtccaccggggacgcgccgccgtcgttcttcccgttcggctggggacctcggacctgcgtcggccagaccttcgcgctgctggaggccaagatagggctcgccatgattctggggagcttcacgttcgagctctccccgtcctactcgcacgcgccgttccccgtcgtcttgctcaagccagagcacggcgcgcaggtcaagctcaggaagctccaatgcatgagcagcatccgcatgcacgctgcacagccgaaacggtcataa

SEQ ID NO: 27 - protein, 569 aa, Zea mays
MATSVPEGLLGEPSSPWKVQQLLPLCAVLAVVVAWCAARAAEWAWLKPRRLERALRAQGIRGTAYRPLAGDAPLSDRLAREARSRPPLPPGCHAIVPRAMPLVHHSMNEHGKTSVTWFGPTPRVIITEPELVRQVLSNKFGHFEKAGFGQLTRLLHYGVSTHEGSKWAKHRRIINPAFHFEKLKRMLPAFASCCADLVSTWERLVVAADGEPCEVDIWPEMQRLTGDVISRVAFGSSYLEGRRIFELQEEQVHLAMLVAGKIHIPGYMMLPTRVNRRMKRIAAEIEGILRRMIVTRESSLRAGKATSDDLLGLLLESNMEQLRGQGEGEGGGGGTSTSSGGLSTDDIIGECKLFYFAGMETTSVLLTWTMLALCMHPEWQDLAREEVLRVFGGASCRVPDYDGLSRLKIVTMVLYEVLRLYTPLPALHRRTYKPMELGGVRYPAGVMLMLPLLCIHHDKDAWGPDADEFRPERFAEGVARASSTGDAPPSFFPFGWGPRTCVGQTFALLEAKIGLAMILGSFTFELSPSYSHAPFPVVLLKPEHGAQVKLRKLQCMSSIRMHAAQPKRS

SEQ ID NO: 28 - DNA, 12655 bp, Zea mays
  source 1..12655: mol_type = genomic DNA
aagccaggtgtcggcgtttcgagactggggggtccctgggccgacgagtgaatgtcgccgcgtgccccagcccagatgggtcggcgcgaggccgagcgcgaaagggggaagtgaggtggccggagacgggcgtgagagaggtggaaatcccgcggccttcgtgttcgtcccgcgcccaggtcgggtgcgcttgcagtagggggttacaagcgtccacgcgggagagggagcgagcggcctcacgcgagcgcctgtctcgtcctcgtccccgcgcggccaaccctgtgtaagagggccctggtccttccttttataggcgtaaggagaggatccaggtgtacaatgggggtgtagcagagtgctacgtgtctagcggaggagagctagcgccctaagtacatgccgtcgtggcagccggagagattttggcacccggttcgtgtggtgtcgtggccgtcggaggagcgctggagcctggtggaaggacagctgtcggggctgtcgagtccttgctgacgtcatcttgcttccgtaagggggcggagagctgccgtcatcagggagcgtgcggggcgccatcattgcctatctggcggagcgagccagatgggacgccggtcttgttccccgtagcctgagtcagcttggggtagggtaatgatgacgcctcctgttgacgtggtcggctcgcgcccgaggttgggcgatgtggaggctcctccgaggttgaggtcgagtctgtcttccgtggccgtggtcgagtccgagcctctgggtcgggcgaggcggagaccgtcggctgaggtcagggctgagtccgagccctggggtcgggcgaagcggagttcgtcgtcttctggggctgagcccaagtccgagccctgggtcgggcggagcggagttcgtcgtcttctggggcttagcccgagtccgagccctgggtcgggcggagcggagttcgccgtcttctagggcttagcccgagtccgagccctgggtcgggcggagcggaattcgccatcttccggggcttagcctgagtccgagccctgggtcgggcggagcggagttcgccgtcttcaggggctgagcccgagtccgagccctgggtcgggcgaagcggagcttcctatggtgcctgcggccgggcctgactgcctgtcagcctcactctgtcaagtggcaccgcagtcggagcggcgcaggcggcgctgtctttctgtcaggccggtcagtggagcggcgaagtgacggcggtcacttcggctctgtcgactgaagggcgcgcgtcaggataaaggtgtcaggccacctttgcattaaatgctcctgcgatttggttggtcggcgcggcgatttggtcagggttgcttcttggcgaagacagggcctcgggcgagccggaagtatgttcgccgctggaggggggcctcgggcgaaacggagatcctccggggtcggctgcccttgtccgaggctaggctcgggcgaggcgtgatcgagtccctcgaatggaccgatcccttacttaatcgcacccatcaggcctttgcagctttatgctgatggggttaccagttgagaattaggaaccttgagggtactcctaattatggtccccgacaccaggcgatggcttctgcaagaaatgtcgtggcttacggtgcaagctagtatatttacaaagggatatttcgacatgcaaaaaaactcgtgccgtttaaaggacaactagaaatgcgacatttgaagtcagaatgcataatataattaaaaaatatatattttcgaaatgaatattatgttacacatgtttgactaaaaaataaattctaaataaattaaaaaaccattttgtgcatgatttatttgaattagcgtgtaatgaaaaaacaaataaatattgttgctttagaagactgaatttaggttgttggagaccgagaaaatatatatgacataattttttataatgtgttgtaaaggatgaaaaatatttatttagggatagaatttaggggctggagagtggagacggtctaagcgtataggacgacggcgatggcgagcagctctctccgttcggttccccttccgctgcacacagaaaagtcacgagcttagatttttttcacccaagtggctggcggctagccaagcgcccaagtcatggcgacgagcgtgccagagggattgctcggcgagccttcttccccgtggaaggtgcagcaactgctgccgctgtgcgcggtcctcgccgtcgtcgtcgcgtggtgtgccgcgcgcgcggcggagtgggcgtggctgaagccgcggcgcctggaacgggcgctgcgggcgcagggtatccgtggcacggcgtaccgcccgctggccggcgacgcgccgctgtccgatcgactcgcccgcgaggccaggtccaggccgcccctaccgccgggctgccacgccatcgtgcccagagccatgccgctcgtccaccactccatgaacgagcacggtaacttctctctcgctgttgtcgcgttgactacttgactctaaccggtgatctacctttagcttgccggtcgtaccacaccaactcactaaggaacgaacggtttttccatggtattacacaaacatttatcatagtaaatttcaaaatataaatcttatgtcatgaagttacataagttatttaataaaaatataaatatttgatttagaataaagctaatacaaagtgtgctgtgggaaatatggtttcaagtagtttgcttgacatcgaggatcattgagttattggtactgattaagatatcaattagcgaataaatttcccctccagtgtggaatattaaaaaaaaaatagagtgcggaggattcaaccctgctcgcttaaggccctttggcacggctccagctcgagcttatagtgaagcctagcaaatggtcaaaaaaatggcttctggtcgatagtcctctaagagctggagtcgttcttttgttgggagcaaaagcataaaaaatatggcttctagcagctcctatatttttctcatacatcattcgaaaaacatatgagaaattattttgccaaacgaattgtaaaatgactttggctccttcagagaagccaaagagccagaggagtcagagccaaaactattttagaggaggcggagccctggcaaagggccatagtataaagcacttggcttgtacaattgcaagtcgtgtttgtttatgtacatgttacgtaaagaagagatattgtaaatatatacatatagtaataatttgaaaataaaaaatataatcatgtttgaataaatatctcaattaagagaggactactccattatatatagaaaccgtagcaataactagttgccatatactttgctagaagttgcatgttattcactaccggatagatctgctttgccgagtggcaaagcccaggaaactctcggtaaaatctttgccgagtgtgactctctgtaaagaagtctcggcgaactgtacatcggcaatggcttctttgctgagtacgttttgtcgggcacttggcaaagaaaagttaatgtcacggcgacaagggacggtgatgcagcctttgtcgagtgtcctctgtgacactcggcaaagaagcgttctttaccgagtgcttgttggcctgacattcggcaaagaaagctctagtgggcctctttgtcagtccctttgtcgagagctctggtaggcactcggtaaagaagctccctttgtcgagtgcctgttggactgccactcagcaaagaaagctccagtgggcctctttgccggttcctttgccgagtgtgctgggaggcactcggcaaagggtgcttatttaccgagtgctgcgaccatatcactcgacaaagaagctttatcggttcccaagtgtcttctttgtcgagtgctttggttatcgcactcggcaaagaacctctttgccgagtgtaacactccggaaattgaccagaaaaaaccttttttatttgtttttattattccatccaaacaaacaaaggatatatatcattgacatcacatatatcacataatcaacacatgtatcaccaacaccataatctcacaaatatcaccacatcaaacaagtttcacaaacataaatctcaccaagtatctcacaacatcaagtattatcaacatcaataagtttaacaaacacaaatatcaccaacactcgcaaacataagtctcaatcatcactaagaggttatcaacaaggtgggcggatggactgttttagcgaagggttgggcaatctgtgagggttatttgatgtcgccgattatccctgcatagagaagagattgcatttgtgagaccagatgaatatatattatgtaggactaaaaatttgatactcacaggagtatggaactgagcaaggtcagctggagggaacaacggaggtggtggagcaagaccctgtgcagcgccaaagctctgcatgtactggaacatctctgccatcctctgctgattcgccaagcgaaccacccgctccatcctcatcctcgcctctatctccatgtgtccctccctcttcttctagctgggcctgtaatatctcaccccaatgttataataatgcaaagagaaggtatataactcaatgaatgacaaggcatagaagcactaacctggagttccaagatacaatgttgggaactatcttgccaaaaggtcgtatggctaggctcgcgctcatactccttgctcacacctgagacagagtgggagtggaggatgagttgattgccccgtcagcaatccagtaccgcccatgcctcttgcctcctccgaccctcatgaggacatctctatcgatgtccttagtcctcggattgtaatctagcccatggacctcctttgccatggcagtgtactcactgaggcggctacggacggtaggttgctataagcctcaggcttgtgctccaggttgtaggtgacatcggacgtcgccttgcccttgtgggccatagcatacgtcgagaagattgagctaggctagccaccatgtgacgtcggctacgataaaaccaacgagatggttagaaatcatgcctaatcaaaagttagactaaattaaagaaagtgcatatacatgcttctatgtatttgccaaggctacgactgccttgatggtgggagggaccttgcatcatcaaactccgttcctggctagcgttgtgcgcctcgtcccactcggccgagcaccacctttgcaccatctgctccaagcactcaggatgcgcgatgcaccaataaggaataatcttcaaagaaaacaattacacaacatatcaaaagataaaattaagcatatttaatatcaatattaatgttctgtatttacctgcaagtactggttcagagtcaatgacatggttcgggcgtccttgctcaccttctccctaaggatggagtcgtggtaagtgatgatggcctggatgcacgcctcgtactgcatgtccacgatgagcttcttatggctcgcggtagccaccacatccatcgtggcctcatttctagcctcgtagctaaagatatcctacgtacaaagacgatgtattcatacattatttttaagaatgtgcaataaatgcaacatattaaacaatatagtgcaaggaagacttacccacagctcttgcttcatccgctccgccttgttgttgaattatctatcatctcgatccactgcatcgggggcgacagtgtagtggtcgaaggaataaaccaagcccatcactccggcatactcaacaagtctaggaaagtgttccctgcacagaagcccgaggatgccattggggttgcggcgtgacccctgcattcttcaaaacctttcaagatatgtctaagtgattaagaaaaattatcagtttcttttttaaatttgaacatataatatgaaaaagtatcgacaacatctaaagttgcatacctttccccatcgggtcgaatgagcgggcacctgtcacgaagtatcggtcgctgaaggagactcgcgagacctcgcaagtagacgtgcatcgaacatgtggtgccagacctagaggcgacccgctgagcgtcttcagtcctactgtgcctcctcctccatcctacgggtgctcctcatcctcctctctctcctcgtcccaccgcccaccatctttgtctaaccacctgcaattaagagtaagcaattaagcctagataaaaaatatgtatttagaaacatatgtgaaataaataaaatatagcattacattgattaaaataatcttcatatgtgttcggattagccagatcataagtctcatcatcattatcaaccatttcatagtcaacatcatatgaaggcacaagtttataattgtaattgccttcaagcatttctaagtcattctcattttgcacttcattttcctcctcatcaacaaccatttcaatatctaccatccatttcgattgctttggttaaatctatctcaaatcgctcttctagctattttcttaaaagaactctctgtcatatgtctctgggtctaagttataatcttcatcgtttagaataggtaatttatcgtgcagtgataccatatacacagcatctcaacccttaagatattcttttatttggcacacatatgggagataatatacttatgtggtctgttggttcacgatatagacatcgtctcctggtaaggtggaatcctatcgaatttcgattagcccaagattagaatgtgtccatctcgtcacttcagcgtcaaaccaatgacatttcagtatgaactgaaggtggcgtaggattgtcacaaggattttgagctgcctcttttgtccatcacccgattctacctctatgaacctagacgatttacacttcagacggtactttgcctctgtgtattttttcctaaataggacacaacccttcagacaaacatgtgtctgctcatacatcatcttgagtgcacgaagaagtttctatgcctcgtacatgctctttggcagaatgtgatcctctagaagcaaactgccaataactatcaacaatccatcaaaggtgtctcgacttaggctgtaatgcgacttgaactccattatacatccaatggcatccagttgagaaacttttgtcttgccatgaaggggtttctgtgccacgtcaaacatgtcgtagaacgcctttgtggtcgcctctagctcgtcctcagtatgtgattcagcgaattatgcctcgtgatagtcgtttaccgctaccttggcaccaacatcataaccctcgatgcgttgtctcgccacctcctctctcgtacgatgcgcttcaccatggaagatccatcgagtatagtctggcgtaaatccattcttccaaatatgttgtctcatggctttctttgtttttcttttcttgttgtcacatttgttgcacggacatggaactagactcacgcctttagcagcttcgccaaatgctcgttccaagaaagcatcagtctttgtaatccattcaggagtgacatcattccttgttacgtggtccatgtacatccactcacgatccttaatactctaacatatataagcaagtatagtctaatataaaaatgtaatgcatatacactatgttcctaccttctaatgggtgaggataggtcctaatcctacccgcagttgcgtagatggggttagtttccatgctctactctgatccaagacagaatttcggcagcacctctccgttgttctatggaaacacatcctagctaggagactgtgtatccagagaacaacagggaggtgatgttgaaactctatctcgaatcggagtagagcatgtaaactaatttcgtctacgtaaccgcgggctatccaaaaaatatggacaattcgaaacatatacacatttgtagatatgtaaagatccacatatctatatcttatctctttcgaacgggagacgcctaactgggttacgtgatctacgaccatgacacagaaatcaaggttataccttaggtggcggtggagtcaggctagcggggctgtggtgcgtcggtgcagtggcaacgtgacacggtgcaggtagacccacaacagctaggaagccctctgcaaaaaaggaaaacaagtctgtcaagacaaaatttcgacagcacctttcctacatggtgatgttttcaaaacctgcaaataaaactaacagcacgatggctgatatccacacatatagatcaaggccttgataaccacatatacatatttgatctcacatattcatgcatgtttgttctcacaattcacaatcacacaaaaaaatagttcactaataataaatttcaaactcatagttcaaaataacttaacacaacttcacaagtcacaatttcacaataacaactaaaaataactaggaccaaacatcaattactagccaaacatcatacaatttctatgtgtgttgcaaatgacaaaaaagagtctttaactttgcttggccggagcacaggagagggcgcagatctaaggtggcggtgtgttagccggagcgggggaccaattgatggggatggtcggagccagcggggacgaggggccggagtcggtgtggacggtggcgccctggtcgatggggacggtggcgcgctggcgctcgagtgggtgacggggccgaagtcggcgtggacggtggcgctggccgacaaggacggtggtgcgctggcgctcgggcgggcgacgcgagagcggtgaggtgctggcgtggcggcgagcgggtggggtgcatggcaacggcaggcagcggttgagcgtgcgaaagaatgggaccacgggctttggtcattgttaagtaaacgttctttgccgagtgctcgcgacctcacactcggcaaagatttttttaaaaatttaaaaatgttactttgccgagtgtcatggatctgacactcggcatagtatactttaccaagtgttctggatctaacactcggcaaagattattttgatttgaaaactactttgttgtgtgttcctctgtagtatccaagacactcggcaaagtatatttttatttttatttttattcttttttcattaaactttttgtggtttgttcctatactatatagacctacatgttccattttagtataattatcaaagtgtttgctataaatattagatttagttcgtttaattgaattttttcggataattcagatttaaacgaaaaatggaaaacagtgaatgcaaaaatgatttcatgttatttagcacaagttacggtctatttcataaacaaaccagaattttcgagcaccatgcttaaacatgaccatcaacctgtgatccagttattttaaaattgtataaaacacaaacaaaatcagcgaatcatgaaacttgtcaacatgtcatgatatcatatatagagaatctgataaaattttaaaactatttcatgaaagttgttacgcactatacgtagaaaatctagctgaagttaatagaagttttatgattttacgtgaagtccgactaattttgactcgtatgactcatgacccatccactctggattgacaacgcgtggcctactggtctgggcactggccaggcaagacctcggtcacctggttcgggccgacgccgagggtgatcataaccgagccggagctggtgcgccaggttctgtccaacaagttcggccacttcgagaaggccggcttcgggcagctgacgcggctgctgcactatggcgtgagcacccacgagggcagcaaatgggccaagcaccgcaggatcatcaaccccgctttccatttcgagaagctcaaggtctcgtctcatgcgtgaatcacctcatcaatagcttgctagttgatggatctgatacgagcttcccgatcgaccgattctcctgcagcgcatgctgccggcctttgcgtcgtgctgcgccgacctggtgagcacatgggagcgtctggtggtcgcggccgacggcgaaccatgcgaggtggacatctggccggagatgcagaggctgacgggggacgtcatctcccgcgtcgccttcggcagcagctacctggaaggcaggaggatcttcgagctccaggaggagcaggtgcacctcgccatgctcgtcgccggcaagatacacatccctggatacatgtcagctcgtgctctctagctagctctgatcaaactacattctatattaatttgattgtcagtgagtaactgacacgacaaaaagattttgatatacagaattgcaattttcgtgcgtgtaggatgctgcctacaagggtaaaccggcggatgaagcggatcgcggcggagatcgaagggatccttagacggatgatcgtgacgagggagagctcgctgagggcaggaaaggcgacgagtgacgaccttctcggcctgctgctagagtccaacatggagcagctcaggggccaaggcgaaggcgaaggcggaggcggcgggacgagcacgagctccggcggcttgtccaccgacgacatcattggagagtgcaagctgttctacttcgccggcatggagaccacgtcggtgctgctcacgtggactatgctggcgctctgcatgcacccggagtggcaggaccttgccagggaggaggtgctccgcgtcttcggcggcgcctcctgcagagtgccggactacgacggcctcagccgactgaaaatcgtaaccttcatttcgatagcaggatcagtcttgatcgattcgtccagtcagtgtcgtgtgaaatgtggtcgtaattgtgattcgcaatcgcgtctgcaggtgaccatggtgctgtacgaggtgctgcggctctacacgccgctgccggcgttgcaccggcggacatacaagcccatggagcttgggggcgtcaggtacccggcgggcgtgatgctgatgctgccgctgctctgcatccaccacgacaaggacgcgtgggggccggacgccgacgagttcaggccggagaggttcgcggagggagtcgccagagcgtcgtccaccggggacgcgccgccgtcgttcttcccgttcggctggggacctcggacctgcgtcggccagaccttcgcgctgctggaggccaagatagggctcgccatgattctggggagcttcacgttcgagctctccccgtcctactcgcacgcgccgttccccgtcgtcttgctcaagccagagcacggcgcgcaggtcaagctcaggaagctccaatgcatgagcagcatccgcatgcacgctgcacagccgaaacggtcataaatttccctgtatatatatgcgaaaaaaggcaacaacatgtatggatggaccaggactattgttagttaatgcctaaaaatactctatgcgtcctaaaataagtatccgtatgtttaaggtgaatagatcaatgtctttaagaaattatatgtccacctataaatatgtaactattgtacttcggaaaaatagaaggtagaaaagaacaactagagttagtttatcttgtaaatcgaggtgcacttattttgtcacagattttaaattctaggagtacacttattttgggatgtacaaagtagtcggctaaaacttgtcaactagttggtagtacataattgtttggattgtcaactaattattagaccttttgtgaacataaggttgatatgtgctcttgtgttttggttgtgatgtatgattatcaacaagagcagtgtctttgggttaaatcgtgtggactaatcggatgtgagagtatttgtgcaatatatctcttgtcttgtggtgtgtaggtttgaagtcttgagatggtagtcgacggcaagggtgaaagtcatgcggaattgcgtcgatggactgggagtagtaaagaacgtacactgggtcttgactgagggactagagaagctagaagactagttatggcggttgacacctgtggacactgataggtgtaaggatatggaggagtggatcgtatcattgacgtggttgtggaccgacggaacacatgtccaagacgtgggatcacgtatgtagtacgctaactatgggggtttaggtgattaagcctcaaaaccacccaacacaagttcgcgggtttcatcggatttttctcaaaactagatggcatggttccgaagggaataggaagcgacatgtggcatcatcatgaatggtgtgccaaggcaaagcaatttcgtgaagagtgtatgataatcatatcaaaattccaagagctagtccattttgcccctatatctagtggtagtttaggatagaggaataaccctttataaataggtaggggctgaaccattcaaccatctctctagcacctctgtaatttgtattctttatttccttctcattttgttaggagtccttagatgctttgaactttaatatccaagttcatgcaattttgaggaaatgtggccataaccacactttgccttttgggttttcaattcgtgtcaaatttaataggttttttctagttattgaggttttgagttgatcttggtcggtactgacccaaacaaccgatcgaccgaccacatgctacgccatattatcaaggatg

SEQ ID NO: 29 - DNA, 1710 bp, Zea mays; identical to SEQ ID NO: 26
  source 1..1710: mol_type = other DNA

SEQ ID NO: 30 - protein, 569 aa, Zea mays; identical to SEQ ID NO: 27

SEQ ID NO: 31 - DNA, 12619 bp, Zea mays
  source 1..12619: mol_type = genomic DNA
gagactggggggtccctgggccgacgagtgaatgtcgccgcgtgccccagcccagatgggtcggcgcgaggccgagcgcgaaagggggaagtgaggtggccggagacgggcgtgagagaggtggaaatcccgcggccttcgtgttcgtcccgcgcccaggtcgggtgcgcttgcagtagggggttacaagcgtccacgcgggagagggagcgagcggcctcacgcgagcgcctgtctcgtcctcgtccccgcgcggccaaccctgtgtaagagggccctggtccttccttttataggcgtaaggagaggatccaggtgtacaatgggggtgtagcagagtgctacgtgtctagcggaggagagctagcgccctaagtacatgccgtcgtggcagccggagagattttggcacccggttcgtgtggtgtcgtggccgtcggaggagcgctggagcctggtggaaggacagctgtcggggctgtcgagtccttgctgacgtcatcttgcttccgtaagggggcggagagctgccgtcatcagggagcgtgcggggcgccatcattgcctatctggcggagcgagccagatgggacgccggtcttgttccccgtagcctgagtcagcttggggtagggtaatgatgacgcctcctgttgacgtggtcggctcgcgcccgaggttgggcgatgtggaggctcctccgaggtcgaggtcgagtctgtcttccgtggccgtggtcgagtccgagcctctgggtcgggcgaggcggagaccgtcggctgaggtcagggctgagtccgagccctggggtcgggcgaagcggagttcgtcgtcttctggggctgagcccaagtccgagccctgggtcgggcggagcggagttcgtcgtcttctggggcttagcccgagtccgagccctgggtcgggcggagcggagttcgccgtcttctagggcttagcccgagtccgagccctgggtcgggcggagcggaattcgccatcttccggggcttagcccgagtccgagccctgggtcgggcggagcggagttcgccgtcttcaggggctgagcccgagtccgagccctgggtcgggcgaagcggagcttcctatggtgcctgcggccgggcctgactgcctgtcagcctcactctgtcaagtggcaccgcagtcggagcggcgcaggcggcgctgtctttctgtcaggccggtcagtggagcggcgaagtgacggcggtcacttcggctctgtcgactgaagggcgcgcgtcaggataaaggtgtcaggccacctttgcattaaatgctcctgcgatttggttggtcggcgcggcgatttggtcagggttgcttcttggcgaagacagggcctcgggcgagccggaagtatgttcgccgctggaggggggcctcgggcgaaacggagatcctccggggtcggctgcccttgtccgaggctaggctcgggcgaggcgtgatcgagtccctcgaatggaccgatcccttacttaatcgcacccatcaggcctttgcagctttatgctgatggggttaccagttgagaattaggaaccttgagggtactcctaattatggtccccgacaccaggcgatggcttctgcaagaaatgtcgtggcttacggtgcaagctagtatatttacaaagggatatttcgacatgcaaaaaaactcgtgccgtttaaaggacaactagaaatgcgacatttgaagtcagaatgcataatataattaaaaaatatatattttcgaaatgaatattatgttacacatgtttgactaaaaaataaattctaaataaattaaaaaaccattttgtgcatgatttatttgaattagcgtgtaatgaaaaaacaaataaatattgttgctttagaagactgaatttaggttgttggagaccgagaaaatatatatgacataattttttataatgtgttgtaaaggatgaaaaatatttatttagggatagaatttaggggctggagagtggagacggtctaagcgtataggacgacggcgatggcgagcagctctctccgttcggttccccttccgctgcacacagaaaagtcacgagcttagatttttttcacccaagtggctggcggctagccaagcgcccaagtcatggcgacgagcgtgccagagggattgctcggcgagccttcttccccgtggaaggtgcagcaactgctgccgctgtgcgcggtcctcgccgtcgtcgtcgcgtggtgtgccgcgcgcgcggcggagtgggcgtggctgaagccgcggcgcctggaacgggcgctgcgggcgcagggtatccgtggcacggcgtaccgcccgctggccggcgacgcgccgctgtccgatcgactcgcccgcgaggccaggtccaggccgcccctaccgccgggctgccacgccatcgtgcccagagccatgccgctcgtccaccactccatgaacgagcacggtaacttctctctcgctgttgtcgcgttgactacttgactctaaccggtgatctacctttagcttgccggtcgtaccacaccaactcactaaggaacgaacggtttttccatggtattacacaaacatttatcatagtaaatttcaaaatataaatcttatgtcatgaagttacataagttatttaataaaaatataaatatttgatttagaataaagctaatacaaagtgtgctgtgggaaatatggtttcaagtagtttgcttgacatcgaggatcattgagttattggtactgattaagatatcaattagcgaataaatttcccctccagtgtggaatattaaaaaaaaaatagagtgcggaggattcaaccctgctcgcttaaggccctttggcacggctccagctcgagcttatagtgaagcctagcaaatggtcaaaaaaatggcttctggtcgatagtcctctaagagctggagtcgttcttttgttgggagcaaaagtataaaaaatatggcttctagcagctcctatatttttctcatacatcattcgaaaaacatatgagaaattattttgccaaacgaattgtaaaatgactttggctccttcagagaagccaaagagccagaggagtcagagccaaaactattttagaggaggcggagccctggcaaagggccatagtataaagcacttggcttgtacaattgcaagtcgtgtttgtttatgtacatgttacgtaaagaagagatattgtaaatatatacatatagtaataatttgaaaataaaaaatataatcatgtttgaataaatatctcaattaagagaggactgctccattatatatagaaaccgtagcaataactagttgccatatactttgctagaagttgcatgttattcactaccggatagatctgctttgccgagtggcaaagcccaggaaactctcggtaaaatctttgccgagtgtgactctctgtaaagaagtctcggcgaactgtacatcggcaatggcttctttgctgagtacgttttgtcgggcacttggcaaagaaaagttaatgtcacggcgacaagggacggtgatgcagcctttgtcgagtgtcctctgtgacactcggcaaagaagcgttctttaccgagtgcttgttggcctgacattcggcaaagaaagctctagtgggcctctttgtcagtccctttgtcgagagctctggtaggcactcggtaaagaagctccctttgtcgagtgcctgttggactgccactcagcaaagaaagctccagtgggcctctttgccggttcctttgtcgagtgtgctgggaggcactcggcaaagggtgcttatttaccgagtgctgcgaccatatcactcgacaaagaagctttatcggttcccaagtgtcttctttgtcgagtgctttggttatcgcactcggcaaagaacctctttgccgagtgtaacactccggaaattgaccagaaaaaaccttttttatttgtttttattattccatccaaacaaacaaaggatatatatcattgacatcacatatatcacataatcaacacatgtatcaccaacaccataatctcacaaatatcaccacatcaaacaagtttcacaaacataaatctcaccaagtatctcacaacatcaagtattatcaacatcaataagtttaacaaacacaaatatcaccaacactcgcaaacataagtctcaatcatcactaagaggttatcaacaaggtgggcggatggactgttttagcgaagggttgggcaatctgtgagggttatttgatgtcgccgattatccctgcatagagaagagattgcatttgtgagaccagatgaatatatattatgtaggactaaaaatttgatactcacaggagtatggaactgagcaaggtcagctggagggaacaacggaggtggtggagcaagaccctgtgcagcgccaaagctctgcatgtactggaacatctctgccatcctctgctgattcgccaagcgaaccacccgctccatcctcatcctcgcctctatctccatgtgtccctccctcttcttctagctgggcctgtaatatctcaccccaatgttataataatgcaaagagaaggtatataactcaatgaatgacaaggcatagaagcactaacctggagttccaagatacaatgttgggaactatcttgccaaaaggtcgtatggctaggctcgcgctcatactccttgctcacacctgagacagagtgggagtggaggatgagttgattgccccgtcagcaatccagtaccgcccatgcctcttgcctcctccgaccctcatgaggacatctctatcgatgtccttagtcctcggattgtaatctagcccatggacctcctttgccatggcagtgtactcactgaggcggctacggacggtaggttgctataagcctcaggcttgtgctccaggttgtaggtgacatcggacgtcgccttgcccttgtgggccatagcatacgtcgagaagattgagctaggctagccaccatgtgacgtcggctacgataaaaccaacgagatggttagaaatcatgcctaatcaaaagttagactaaattaaagaaagtgcatatacatgcttctatgtatttgccaaggctacgactgccttgatggtgggagggaccttgcatcatcaaactccgttcctggctagcgttgtgcgcctcgtcccactcggccgagcaccacctttgcaccatctgctccaagcactcaggatgcgcgatgcaccaataaggaataatcttcaaagaaaacaattacacaacatatcaaaagataaaattaagcatatttaatatcaatattaatgttctgtatttacctgcaagtactggttcagagtcaatgacatggttcgggcgtccttgctcaccttctccctaaggatggagtcgtggtaagtgatgatggcctggatgcacgcctcgtactgcatgtccacgatgagcttcttatggctcgcggtagccaccacatccatcgtggcctcatttctagcctcgtagctaaagatatcctacgtacaaagacgatgtattcatacattatttttaagaatgtgcaataaatgcaacatattaaacaatatagtgcaaggaagacttacccacagctcttgcttcatccgctccgccttgttgttgaattatctatcatctcgatccactgcatcgggggcgacagtgtagtggtcgaaggaataaaccaagcccatcactccggcatactcaacaagtctaggaaagtgttccctgcacagaagcccgaggatgccattggggttgcggcgtgacccctgcattcttcaaaacctttcaagatatgtctaagtgattaagaaaaattatcagtttcttttttaaatttgaacatataatatgaaaaagtatcgacaacatctaaagttgcatacctttccccatcgggtcgaatgagcgggcacctgtcacgaagtatcggtcgctgaaggagactcgcgagacctcgcaagtagacgtgcatcgaacatgtggtgccagacctagaggcgacccgctgagcgtcttcagtcctactgtgcctcctcctccatcctacgggtgctcctcatcctcctctctctcctcgtcccaccgcccaccatctttgtctaaccacctgcaattaagagtaagcaattaagcctagataaaaaatatgtatttagaaacatatgtgaaataaataaaatatagcattacattgattaaaataatcttcatatgtgttcggattagccagatcataagtctcatcatcattatcaaccatttcatagtcaacatcatatgaaggcacaagtttataattgtaattgccttcaagcatttctaagtcattctcattttgcacttcattttcctcctcatcaacaaccatttcaatatctaccatccatttcgattgctttggttaaatctatctcaaatcgctcttctagctattttcttaaaagaactctctgtcatatgtctctgggtctaagttataatcttcatcgtttagaataggtaatttatcgtgcagtgataccatatacacagcatctcaacccttaagatattcttttatttggcacacatatgggagataatatacttatgtggtctgttggttcacgatatagacatcgtctcctggtaaggtggaatcctatcgaatttcgattagcccaagattagaatgtgtccatctcgtcacttcagcgtcaaaccaatgacatttcagtatgaactgaaggtggcgtaggattgtcacaaggattttgagctgcctcttttgtccatcacccgattctacctctatgaacctagacgatttacacttcagacggtactttgcctctgtgtattttttcctaaataggacacaacccttcagacaaacatgtgtctgctcatacatcatcttgagtgcacgaagaagtttctatgcctcgtacatgctctttggcagaatgtgatcctctagaagcaaactgccaataactatcaacaatccatcaaaggtgtctcgacttaggctgtaatgcgacttgaactccattatacatccaatggcatccagttgagaaacttttgtcttgccatgaaggggtttctgtgccacgtcaaacatgtcgtagaacgcctttgtggtcgcctctagctcgtcctcagtatgtgattcagcgaattatgcctcgtgatagtcgtttaccgctaccttggcaccaacatcataaccctcgatgcgttgtctcgccacctcctctctcgtacgatgcgcttcaccatggaagatccatcgagtatagtctggcgtaaatccattcttccaaatatgttgtctcatggctttctttgtttttcttttcttgttgtcacatttgttgcacggacatggaactagactcacgcctttagcagcttcgccaaatgctcgttccaagaaagcatcagtctttgtaatccattcaggagtgacatcattccttgttacgtggtccatgtacatccactcacgatccttaatactctaacatatataagcaagtatagtctaatataaaaatgtaatgcatatacactatgttcctaccttctaatgggtgaggataggtcctaatcctacccgcagttgcgtagatggggttagtttccatgctctactctgatccaagacagaatttcggcagcacctctccgttgttctatggaaacacatcctagctaggagactgtgtatccagagaacaacagggaggtgatgttgaaactctatctcgaatcggagtagagcatgtaaactaatttcgtctacgtaaccgcgggctatccaaaaaatatggacaattcgaaacatatacacatttgtagatatgtaaagatccacatatctatatcttatctctttcgaacgggagacgcctaactgggttacgtgatctacgaccatgacacagaaatcaaggttataccttaggtggcggtggagtcaggctagcggggctgtggtgcgtcggtgcagtggcaacgtgacacggtgcaggtagacccacaacagctaggaagccctctgcaaaaaaggaaaacaagtctgtcaagacaaaatttcgacagcacctttcctacatggtgatgttttcaaaacctgcaaataaaactaacagcacgatggctgatatccacacatatagatcaaggccttgataaccacatatacatatttgatctcacatattcatgcatgtttgttctcacaattcacaatcacacaaaaaaatagttcactaataataaatttcaaactcatagttcaaaataacttaacacaacttcacaagtcacaatttcacaataacaactaaaaataactaggaccaaacatcaattactagccaaacatcatacaatttctatgtgtgttgcaaatgacaaaaaagagtctttaactttgcttggccggagcacaggagagggcgcagatctaaggtggcggtgtgttagccggagcgggggaccaatttatggggatggtcggagccagcggggacgaggggccggagtcggtgtggacggtggcgccctggtcgatggggacggtggcgcgctggcgctcgagtgggtgacggggccgaagtcggcgtggacggtggcgctggccgacaaggacggtggtgcgctggcgctcgggcgggcgacgcgagagcggtgaggtgctggcgtggcggcgagcgggtggggtgcatggcaacggcaggcagcggttgagcgtgcgaaagaatgggaccacgggctttggtcattgttaagtaaacgttctttgccgagtgctcgcgacctcacactcggcaaagattttttttaaaatttaaaaatgttactttgccgagtgccatggatctgacactcggcatagtatactttaccaagtgttctggatctaacactcggcaaagattattttgatttgaaaactactttgttgtgtgttcctctgtagtatccaagacactcggcaaagtatatttttatttttatttttattcttttttcattaaactttttgtggtttgttcctatactatatagacctacatgttccattttagtataattatcaaagtgtttgctataaatattagatttagttcgtttaattgaattttttcggataattcagatttaaacgaaaaatggaaaacagtgaatgcaaaaatgatttcatgttatttagcacaagttacggtctatttcataaacaaaccagaattttcgagcaccatgcttaaacatgaccatcaacctgtgatccagttattttaaaattgtataaaacacaaacaaaatcagcgaatcatgaaacttgtcaacatgtcatgatatcatatatagagaatctgataaaattttaaaactatttcatgaaagttgttacgcactatacgtagaaaatctagctgaagttaatagaagttttatgattttacgtgaagtccgactaattttgactcgtatgactcatgacccatccactctggattgacaacgcgtggcctactggtctgggcactggccaggcaagacctcggtcacctggttcgggccgacgccgagggtgatcataaccgagccggagctggtgcgccaggttctgtccaacaagttcggccacttcgagaaggccggcttcgggcagctgacgcggctgctgcactatggcgtgagcacccacgagggcagcaaatgggccaagcaccgcaggatcatcaaccccgctttccatttcgagaagctcaaggtctcgtctcatgcgtgaatcacctcatcaatagcttgctagttgatggatctgatacgagcttcccgatcgaccgattctcctgcagcgcatgctgccggcctttgcgtcgtgctgcgccgacctggtgagcacatgggagcgtctggtggtcgcggccgacggcgaaccatgcgaggtggacatctggccggagatgcagaggctgacgggggacgtcatctcccgcgtcgccttcggcagcagctacctggaaggcaggaggatcttcgagctccaggaggagcaggtgcacctcgccatgctcgtcgccggcaagatacacatccctggatacatgtcagctcgtgctctctagctagctctgatcaaactacattctatattaatttgattgtcagtgagtaactgacacgacaaaaatattttgatatacagaattgcaattttcgtgcgtgtaggatgctgcctacaagggtaaaccggcggatgaagcggatcgcggcggagatcgaagggatccttagacggatgatcgtgacgagggagagctcgctgagggcaggaaaggcgacgagtgacgaccttctcggcctgctgctagagtccaacatggagcagctcaggggccaaggcgaaggcgaaggcggaggcggcgggacgagcacgagctccggcggcttgtccaccgacgacatcattggagagtgcaagctgttctacttcgccggcatggagaccacgtcggtgctgctcacgtggactatgctggcgctctgcatgcacccggagtggcaggaccttgccagggaggaggtgctccgcgtcttcggcggcgcctcctgcagagtgccggactacgacggcctcagccgactgaaaatcgtaaccttcatttcgatagcaggatcagtcttgatcgattcgtccagtcagtgtcgtgtgaaatgtggtcgtaattgtgattcgcaatcgcgtctgcaggtgaccatggtgctgtacgaggtgctgcggctctacacgccgctgccggcgttgcaccggcggacatacaagcccatggagcttgggggcgtcaggtacccggcgggcgtgatgctgatgctgccgctgctctgcatccaccacgacaaggacgcgtgggggccggacgccgacgagttcaggccggagaggttcgcggagggagtcgccagagcgtcgtccaccggggacgcgccgccgtcgttcttcccgttcggctggggacctcggacctgcgtcggccagaccttcgcgctgctggaggccaagatagggctcgccatgattctggggagcttcacgttcgagctctccccgtcctactcgcacgcgccgttccccgtcgtcttgctcaagccagagcacggcgcgcaggtcaagctcaggaagctcccatgagctgcatccgcttgcatattgcagagacgaaacggtcataaatttccctgtatatatacggaaaaaaaaggcaacaacatgtttgctgtttggatggaccaggactattgttagtttatgcctaaaaatactctatatgcgtcctaaaataagtattcgtaggtttaaggtgaatagataattgtctttaagaaattacatgtccccctataaagatgtaactattgtactttggaaaaggagaaggtagaaaagaacaactagagttagtttaacttgtacctggaggtgcacttattttgtcataatttttttaaattctaggagtacacttttttttgggatgtagagtagtccgctaaaatttactcaactagttgttagtccataattatttggattgtgagccaattattaggcattttgcaaacataaggtgtgtttcgattgtgacgagtaagtagtgtcttggattgagatgggtggactaattgggcatgagagtatttgtacaacacatctcttggcttgtagcgtggagttgaaaatggtgatacgaagataaaagtcatgtggattcttgtcaatggatcgaaaacggtaaggataagcactaggacttgatcgaacactaacatgtgcaaggatattagagcagatcataccaccgacacggttacgttacacactaacagaacacgtgtctaaaacatgggtcacgtatgaagttttcatacactacccacaagggtttcgatggtaaacctcaaaaccactcaacactggttaaacctcaaaaccactcaacacgccagtccacgggttttgtctttaagacccgatgacatagttccgaaggggtccaagcggcccctaacagaattgcgtaggaaaactcatttttcgataatcacgtatatcacgactcaccgcgggtccaccggtaaatggcactcatcaggcccaaatcgaagcataatttgattggtgaactaggctctgcttgtctacgcacaaaactgatgaggacatcaaaacttacaattcagtctaagtctagtgacatcatacctactacagtttatagaagtagaaaaggcaacagaaagaaatgtaaacctactgcagtttatggattctagctgctattcccaaataaattctcaaacaacttcggagccattggaggaagaaccatgggctccggtgccatgaaattagtgatcttctcgtggacatggtacctgaaaaaatcacataacagaaatgagcataataccaaaaggggagttattggaaaattttcaaacgtcagcaaaccgcagaggatagagatcg

SEQ ID NO: 32 - DNA, 1665 bp, Zea mays
  source 1..1665: mol_type = other DNA
atggcgacgagcgtgccagagggattgctcggcgagccttcttccccgtggaaggtgcagcaactgctgccgctgtgcgcggtcctcgccgtcgtcgtcgcgtggtgtgccgcgcgcgcggcggagtgggcgtggctgaagccgcggcgcctggaacgggcgctgcgggcgcagggtatccgtggcacggcgtaccgcccgctggccggcgacgcgccgctgtccgatcgactcgcccgcgaggccaggtccaggccgcccctaccgccgggctgccacgccatcgtgcccagagccatgccgctcgtccaccactccatgaacgagcacggcaagacctcggtcacctggttcgggccgacgccgagggtgatcataaccgagccggagctggtgcgccaggttctgtccaacaagttcggccacttcgagaaggccggcttcgggcagctgacgcggctgctgcactatggcgtgagcacccacgagggcagcaaatgggccaagcaccgcaggatcatcaaccccgctttccatttcgagaagctcaagcgcatgctgccggcctttgcgtcgtgctgcgccgacctggtgagcacatgggagcgtctggtggtcgcggccgacggcgaaccatgcgaggtggacatctggccggagatgcagaggctgacgggggacgtcatctcccgcgtcgccttcggcagcagctacctggaaggcaggaggatcttcgagctccaggaggagcaggtgcacctcgccatgctcgtcgccggcaagatacacatccctggatacatgatgctgcctacaagggtaaaccggcggatgaagcggatcgcggcggagatcgaagggatccttagacggatgatcgtgacgagggagagctcgctgagggcaggaaaggcgacgagtgacgaccttctcggcctgctgctagagtccaacatggagcagctcaggggccaaggcgaaggcgaaggcggaggcggcgggacgagcacgagctccggcggcttgtccaccgacgacatcattggagagtgcaagctgttctacttcgccggcatggagaccacgtcggtgctgctcacgtggactatgctggcgctctgcatgcacccggagtggcaggaccttgccagggaggaggtgctccgcgtcttcggcggcgcctcctgcagagtgccggactacgacggcctcagccgactgaaaatcgtgaccatggtgctgtacgaggtgctgcggctctacacgccgctgccggcgttgcaccggcggacatacaagcccatggagcttgggggcgtcaggtacccggcgggcgtgatgctgatgctgccgctgctctgcatccaccacgacaaggacgcgtgggggccggacgccgacgagttcaggccggagaggttcgcggagggagtcgccagagcgtcgtccaccggggacgcgccgccgtcgttcttcccgttcggctggggacctcggacctgcgtcggccagaccttcgcgctgctggaggccaagatagggctcgccatgattctggggagcttcacgttcgagctctccccgtcctactcgcacgcgccgttccccgtcgtcttgctcaagccagagcacggcgcgcaggtcaagctcaggaagctcccatga

SEQ ID NO: 33 - protein, 554 aa, Zea mays
MATSVPEGLLGEPSSPWKVQQLLPLCAVLAVVVAWCAARAAEWAWLKPRRLERALRAQGIRGTAYRPLAGDAPLSDRLAREARSRPPLPPGCHAIVPRAMPLVHHSMNEHGKTSVTWFGPTPRVIITEPELVRQVLSNKFGHFEKAGFGQLTRLLHYGVSTHEGSKWAKHRRIINPAFHFEKLKRMLPAFASCCADLVSTWERLVVAADGEPCEVDIWPEMQRLTGDVISRVAFGSSYLEGRRIFELQEEQVHLAMLVAGKIHIPGYMMLPTRVNRRMKRIAAEIEGILRRMIVTRESSLRAGKATSDDLLGLLLESNMEQLRGQGEGEGGGGGTSTSSGGLSTDDIIGECKLFYFAGMETTSVLLTWTMLALCMHPEWQDLAREEVLRVFGGASCRVPDYDGLSRLKIVTMVLYEVLRLYTPLPALHRRTYKPMELGGVRYPAGVMLMLPLLCIHHDKDAWGPDADEFRPERFAEGVARASSTGDAPPSFFPFGWGPRTCVGQTFALLEAKIGLAMILGSFTFELSPSYSHAPFPVVLLKPEHGAQVKLRKLP

SEQ ID NO: 34 - DNA, 101 bp, Zea mays
  source 1..101: mol_type = genomic DNA
ataaagagcacaaataatgacatcgattgaaaatacttaaacaacatggtrgtagaaaaataccgtacggtgcagttttttagaaagtaacagttggcact